(19) United States
(12) Patent Application Publication
(10) Pub. No.: US 2012/0238546 A1
(43) Pub. Date: Sep. 20, 2012

(54) GAMMA SECRETASE MODULATORS

(76) Inventors: Zhaoning Zhu, Plainsboro, NJ (US); Xianhai Huang, Warren, NJ (US); Anandan Palani, Bridgewater, NJ (US); William J. Greenlee, Teaneck, NJ (US); Robert G. Aslanian, Rockaway, NJ (US)

(21) Appl. No.: 13/378,080

(22) PCT Filed: Jun. 15, 2010
(86) PCT No.: PCT/US10/38649

§ 371 Date: Jun. 4, 2012

Related U.S. Application Data
(60) Provisional application No. 61/187,467, filed on Jun. 16, 2009.

(51) Int. Cl. A61K 31/553; A61P 25/28; A61K 31/551; C07D 498/04; C07D 471/04

(52) U.S. Cl. 514/211.08; 540/545; 540/554; 540/567

(57) ABSTRACT

This invention provides novel compounds that are modulators of gamma secretase. The compounds have the Formula (I): Also disclosed are methods of modulating gamma secretase activity and methods of treating Alzheimer's Disease using the compounds of Formula (I).

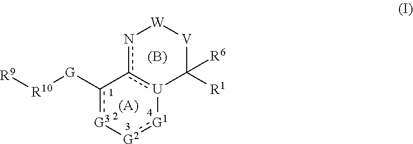

### FIELD OF THE INVENTION

The present invention relates to certain heterocyclic compounds useful as gamma secretase modulators (including inhibitors, antagonists and the like), pharmaceutical compositions comprising the compounds, and methods of treating various diseases using the compounds and compositions. Examples of the diseases and conditions include, for example, Alzheimers disease, mild cognitive impairment (MCI), Downs Syndrome, Glaucoma, Cerebral amyloid angiopathy, stroke or dementia, Microgliosis and brain inflammation, and Olfactory function loss.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute the main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249).

Furthermore, it is known that mutations of APP and presenelin genes, which are observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and normeuronal cells, Journal of Biological Chemistry, Dec. 19, 1997, 272(51), p. 32247-32253). Therefore, compounds which reduce production of Aβ40 and Aβ42 are expected to be agents for controlling progress of Alzheimer's disease or for preventing the disease.

These Aβs are produced when APP is cleaved by beta secretase and subsequently cleaved by gamma secretase. In consideration of this, creation of inhibitors of γ-secretase and β-secretase has been attempted for the purpose of reducing production of Aβs. Many of these known secretase inhibitors are peptides or peptidomimetics such as L-685,458. L-685,458, an aspartyl protease transition state mimic, is a potent inhibitor of γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics™, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

This invention provides novel compounds that are gamma secretase modulators, of the Formula I:

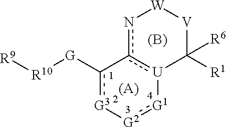

or a pharmaceutically acceptable salt or solvate thereof, wherein all substituents are defined below.

This invention also provides compounds of Formula I.

This invention also provides compounds of Formula I in pure and isolated form.

This invention also provides compounds of Formula I selected from the group consisting of: compounds of formulas 1 through 4, 1.0 through 11.0, 1.1 through 11.1 herein.

This invention also provides pharmaceutical compositions comprising an effective amount of one or more (e.g., one) compounds of Formula I, or a pharmaceutically acceptable salt, ester or solvate thereof, and a pharmaceutically acceptable carrier.

This invention also provides pharmaceutical compositions comprising an effective amount of one or more (e.g., one) compounds of Formula I, or a pharmaceutically acceptable salt, ester or solvate thereof, and an effective amount of one or more (e.g., one) other pharmaceutically active ingredients (e.g., drugs), and a pharmaceutically acceptable carrier.

The compounds of Formula I can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of Formula I to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of Formula I and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of Formula I to a patient in need of such treatment.

This invention also provides a kit comprising, in separate containers, in a single package, pharmaceutical compositions for use in combination, wherein one container comprises an effective amount of a compound of Formula I in a pharmaceutically acceptable carrier, and another container (i.e., a second container) comprises an effective amount of another pharmaceutically active ingredient (as described below), the combined quantities of the compound of Formula I and the other pharmaceutically active ingredient being effective to treat the diseases or conditions mentioned in any of the above methods.

This invention also provides any of the above mentioned methods, pharmaceutical compositions or kit wherein the compound of Formula I is selected from the group consisting of a compound of the Formula I through 4, 1.0 through 11.0, 1.1 through 11.1 herein.

### DETAILED DESCRIPTION OF THE INVENTION

This invention provides compounds, useful as gamma secretase modulators, of Formula I:

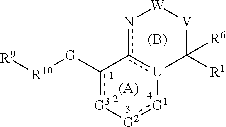

or pharmaceutically acceptable salt, solvate or prodrug of said compound, wherein:

G, G¹, G², G³, R¹, R⁹, R¹⁰, R²¹, U, V and W are each independently selected;

(A) and (B) in the rings in Formula 1 are reference letters to identify the ring; either ring (A) is a 5 to 8-membered aryl-, heteroaryl-, cycloalkyl-, cycloalkenyl-, heterocyclyl- or heterocyclenyl-ring, and ring (B) is a 7 or 8-membered heterocyclyl- or heterocyclenyl-ring which contains at least one N atom, or ring (A) is a 7 or 8-membered cycloalkyl-, cycloalkenyl-, heterocyclyl- or heterocyclenyl-ring, and ring (B) is a 5 to 8-membered heterocyclyl- or heterocyclenyl-ring which contains at least one N atom;

each dotted line (----) represents an optional bond;

U is selected from the group consisting of: N, C (if the optional double bond attached to U is present), —CH— or —CR²¹—;

G¹ is selected from the group consisting of: a direct bond, —O—, —C(R²¹)₂—, —CHR²¹—, —CH₂—, —CR²¹— (if the optional double bond to G¹ is present), CH (if the optional double bond to G¹ is present), —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —N═ (if the optional double bond to G¹ is present), —C(O)—, —C(═NR²)—, —S(O)₂— and —S(O)—, with the proviso that when the optional double bond between (3) and (4) is present then G¹ is not —O—, —OC(R²¹)₂—, —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —C(O)—, —C(═NR²)—, —S(O)₂—, or —S(O)—;

G² is selected from the group consisting of: —O—, —OC(R²¹)₂—, —CHR²¹—, —CH₂—, —CR²¹— (if the optional double bond to G² is present), CH (if the optional double bond to G² is present), —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —N═ (if the optional double bond to G² is present), —C(O)—, —C(═NR²)—, —S(O)₂— and —S(O)—, with the proviso that when the optional double bond between (3) and (4) is present then G² is not —O—, —C(R²¹)₂—, —CHR²¹—, —CH₂—, —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —C(O)—, —C(═NR²)—, —S(O)₂—, or —S(O)—;

G³ is selected from the group consisting of: —OC(R²¹)₂—, —CHR²¹—, —CH₂—, —CR²¹— (if the optional double bond to G³ is present), CH (if the optional double bond to G³ is present), —NR²—, and —N═ (if the optional double bond to G³ is present), with the proviso that when the optional double bond between (1) and (2) is present then G³ is not —OC(R²¹)₂—, —CHR²¹—, —CH₂—, or —NR²—;

W is selected from the group consisting of: —O—, —S—, —OC(R²¹)₂—, —CHR²¹—, —CH₂—, —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —C(O)—, —C(═NR²)—, —S(O)₂— and —S(O)—;

V is selected from the group consisting of: a direct bond, —O—, —C(R²¹)₂—, —CHR²¹—, —CH₂—, —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —C(O)—, —C(═NR²)—, —S(O)₂— and —S(O)—;

G is selected from the group consisting of: —C(O)—, —(C═NR²)—, —(C═C(R⁶)₂)—, —C(R⁴)₂—, —CF₂—, —N(R²)— (and in one example, —NH—), —O—, —S—, —S(O)ₜ, —CR⁴(OH)—, —CR⁴(OR⁴)—, —C═C—, alkynyl, —C(O)NR⁵—, —O—C(O)—NR⁵—, —NR⁵C(O)—, —NR⁵S(O)ₜ—, —S(O)ₜNR⁵—, —NR⁵—C(O)—O—, —NR⁵—C(O)—NR⁵—, —NR⁵—S(O)ₜ—NR⁵—, —NR⁵—C(═NR²)—NR⁵—, —NR⁵—C(═NR²)—O—, —O—C(═NR²)—NR⁵—, —C(R⁴)═N—O—, —O—N═C(R⁴)—, —O—C(R⁴)═N—, —N═C(R⁴)—O—, —(CH₂)₂₋₃— (i.e., 2 to 3 —CH₂— groups), cycloalkyl, and heterocycloalkyl (comprising 1 to 4 heteroatoms independently selected from the group consisting of: —O—, —NR²—, —S—, —S(O)—, and —S(O)₂);

R¹ is selected from the group consisting of: alkyl, alkenyl, alkynyl, cycloalkyl, cycloalkylalkyl-, heterocyclyl (e.g., heterocycloalkyl), cycloalkenyl, arylalkyl-, alkylaryl-, aryl (e.g., phenyl), heteroaryl (e.g., pyridyl), heterocyclenyl (i.e., heterocycloalkenyl), fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl (i.e., cycloalkylusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), and fused heterocycloalkylheteroarylalkyl- (i.e., heterocycloalkylfusedheteroarylalkyl-), and wherein each of said alkyl, alkenyl, alkynyl, cycloalkyl, heterocyclyl, cycloalkenyl, aryl, heteroaryl, heterocyclenyl, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups;

R⁶ is selected from the group consisting of: H, —C(O)R¹⁵, —C(O)OR¹⁵, —C(O)N(R¹⁵)(R¹⁶), —C(═NOR¹⁵)R¹⁶, —CN, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, benzofusedcycloalkyl (i.e., fused benzocycloalkyl), fused benzoheterocycloalkyl, fused heteroarylcycloalkyl, and fused heteroarylheterocycloalkyl; and wherein each of said R⁶ alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl-, benzofusedcycloalkyl, fused benzoheterocycloalkyl, fused heteroarylcycloalkyl, and fused heteroarylheterocycloalkyl group is optionally substituted with 1-5 independently selected R²¹ substituents; or

R¹ and R⁶, taken together with the carbon atom to which they are bound, form a spirocyclic carbocyclic moiety or a spirocyclic heterocyclic moiety, and:

(a) optionally, said spirocyclic carbocyclic moiety is substituted with 1-4 independently selected R²¹ substituents,

(b) optionally, said spirocyclic heterocyclic moiety is substituted with 1-4 independently selected R²¹ substituents,

(c) optionally, said spirocyclic carbocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents,

(d) optionally, said spirocyclic heterocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents;

R² is selected from the group consisting of: H, —OH, —O-alkyl (i.e., alkoxy), —O-(halo substituted alky) (such as, for example, —O-fluoroalkyl), —NH(R⁴), —N(R⁴)₂ (wherein each R⁴ is independently selected), —NH₂, —S(R⁴), —S(O)R⁴, —S(O)(OR⁴), —S(O)₂R⁴, —S(O)₂(OR⁴), —S(O)NHR⁴, —S(O)N(R⁴)₂ (wherein each R⁴ is independently selected), —S(O)NH₂, —S(O)₂NHR⁴, —S(O)₂N(R⁴)₂ (wherein each R⁴ is independently selected), —S(O)₂NH₂, —CN, —C(O)OR⁴, —C(O)NHR⁴, —C(O)N(R⁴)₂ (wherein each R⁴ is independently selected), —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

R³ is selected from the group consisting of: H, —OH, halo, —O-alkyl (i.e., alkoxy), —O-(halo substituted alkyl), —NH(R⁴), —N(R⁴)₂ (wherein each R⁴ is independently selected), —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)OR⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

each R⁴ is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

each R⁵ is independently selected from the group consisting of: H, unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted aryl, substituted aryl, unsubstituted heteroaryl and substituted heteroaryl; wherein said substituted groups are substituted with 1 to 5 substituents independently selected from R²¹;

R⁹ is selected from the group consisting of: aryl, heteroaryl, heterocyclyl, heterocyclenyl, and heterocyclyalkyl-, wherein each of said R⁹ aryl, heteroaryl, heterocyclyl, heterocyclenyl, or heterocyclyalkyl- is optionally substituted with 1 to 5 independently selected R²¹ groups;

R¹⁰ is selected from the group consisting of: aryl-, heteroaryl-, fused cycloalkylaryl (i.e., cycloalkylfusedaryl-), fused heterocycloalkylaryl- (i.e., heterocycloalkylfusedaryl-), fused cycloalkylheteroaryl- (i.e., cycloalkylfusedheteroaryl-), and fused heterocycloalkylheteroaryl- (i.e., heterocycloalkylfusedheteroaryl-), wherein each of said R¹⁰ moieties is optionally substituted with 1 to 5 independently selected R²¹ groups;

optionally, R⁹ and R¹⁰ can be linked together to form a fused tricyclic alkyl ring, a fused tricyclic heteroalkyl ring, a fused tricyclic aryl ring, a fused tricyclic heteroaryl ring, a fused tricyclic alkenyl ring or a fused tricyclic heteroalkenyl ring;

R¹⁵, R¹⁶ and R¹⁷ are the same or different and each is independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

R^{15A} and R^{16A} are the same or different and each is independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

optionally, two R¹⁸ moieties on adjacent carbons can be linked together to form a

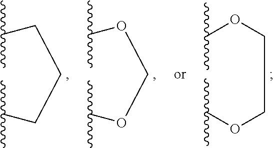

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —P(O)(CH₃)₂, —SO(═NR¹⁵)R¹⁶—, —SF₅, —OSF₅, —Si(R¹⁵)₃ wherein each R¹⁵ is independently selected, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —N₃, —NO₂, —S(O)₂R^{15A}, —O—N═C(R⁴)₂ (wherein each R⁴ is independently selected), and —O—N═C(R⁴)₂ (wherein the two R⁴ groups are taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—), wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;

each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R¹⁵)₃ wherein each R¹⁵ is independently selected, —SR⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A} —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A};

optionally, one of (a)-(b) below can represent a spiro ring:

(a) when G¹, G² or G³ represents —C(R²¹)₂—, —C(R²¹)₂—C(R²¹)₂—, —CHR²¹—C(R²¹)₂—, —C(R²¹)₂—CHR²¹—, —C(R²¹)₂—CH₂—, or —CH₂—C(R²¹)₂—, two R²¹ groups on one of the —C(R²¹)₂— groups on one of G¹, G² or G³ can together form a 3 to 8-membered cycloalkyl, cycloalkenyl, heterocycloalkyl, and heterocycloalkenyl spiro ring attached to ring (A), or

(b) when W or V represents —C(R²¹)₂—, —C(R²¹)₂—C(R²¹)₂—, —CHR²¹—C(R²¹)₂—, —C(R²¹)₂—CHR²¹—, —C(R²¹)₂—CH₂—, or —CH₂—C(R²¹)₂—, two R²¹ groups on one of the —C(R²¹)₂— groups on one of W or V can together form a 3 to 8-membered cycloalkyl, cycloalkenyl, heterocycloalkyl, and heterocycloalkenyl spiro ring attached to ring (B);

d is 0 or 1;

m is 0 to 6;

n is 1 to 5;

p is 0 to 5;

r is 1 to 3;

t is 1 or 2; and

provided that:
(a) rings (A) and (B) do not have two adjacent —O— atoms in the ring; and
(b) rings (A) and (B) do not have two adjacent sulfur groups in the ring (i.e., when there is a —S—, —S(O)— or —S(O)₂ group at one position, then the adjacent positions are not —S—, —S(O)— or —S(O)₂); and
(c) rings (A) and (B) do not have an —O— atom adjacent to a sulfur group (i.e., rings (A) and (B) do not have an —O— atom adjacent to a —S—, —S(O)— or —S(O)₂); and
(d) when G¹ is N, then G² is not —O—; and
(e) when G¹ is —O—, then G² is not N; and
(f) when G¹ is N, then G² is not —S—; and
(g) when G¹ is N, and G³ is N, then G² is not N; and
(h) when G² is N, and G³ is N, then G¹ is not N; and
(i) when G¹ is N, and G² is N, then G³ is not N; and
(j) when the double bond between positions 1 and 2 is not present, then G is not —C(O)—, —(C═NR²)— or —(C═C(R⁴)₂)—.

Preferably, the compounds of the invention are of formula Ia:

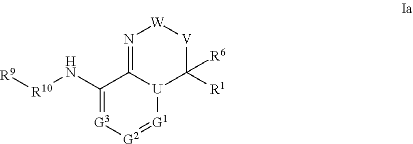

wherein R⁹, R¹⁰, G¹, G², G³, W, V, R⁶ and R¹ are as defined above for Formula 1. More preferably, the compounds of the invention are of formula Ib:

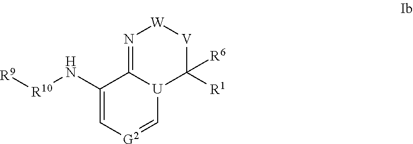

wherein R⁹, R¹⁰, G², W, V, R⁶ and R¹ are as defined above for Formula 1.

In one embodiment, R¹⁰ is selected from the group consisting of

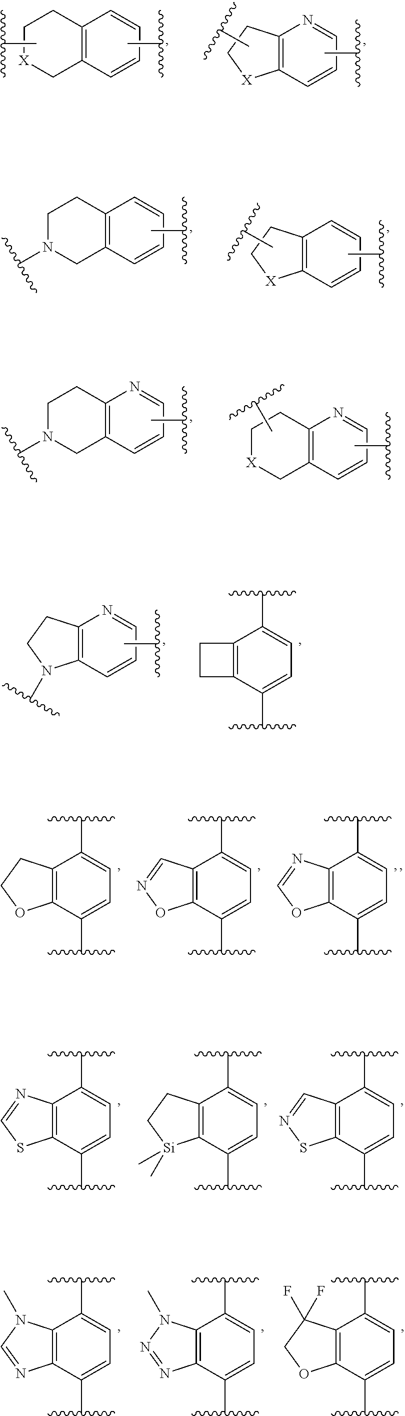

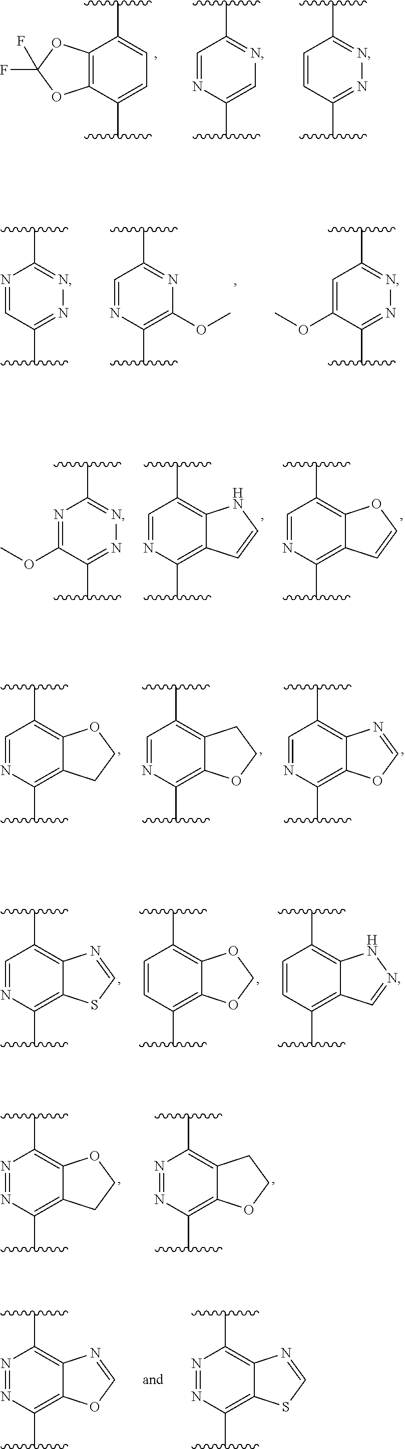

wherein X is selected from the group consisting of: O, N(R¹⁴) or S, wherein R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), and wherein, optionally, each of said R¹⁰ groups are substituted with 1-3 independently selected R²¹ substitutents.

Preferred embodiments in which R⁹ and R¹⁰ are linked together to form a fused tricyclic ring are selected from the group consisting of:

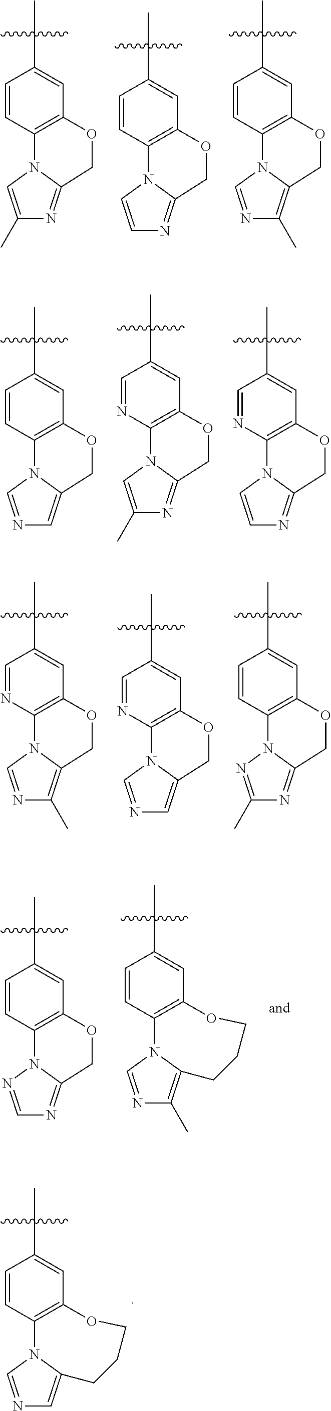

Those skilled in the art will appreciate that the moiety:

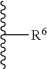

can have the stereochemistry

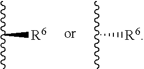

The moiety

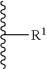

can have the stereochemistry

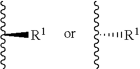

Thus, in one embodiment of this invention the R⁶ and R¹ moieties can have the stereochemistry:

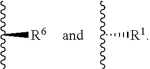

And in another embodiment of this invention the R⁶ and R¹ moieties can have the stereochemistry:

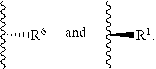

The R⁶ benzofusedcycloalkyl (i.e., fused benzocycloalkyl), fused benzoheterocycloalkyl, fused heteroarylcycloalkyl, and fused heteroarylheterocycloalkyl groups, can be optionally substituted with 1-5 independently selected R²¹ groups. In one example, the R²¹ groups are halo (e.g., F).

Examples of the fused ring R⁶ groups include, but are not limited to:

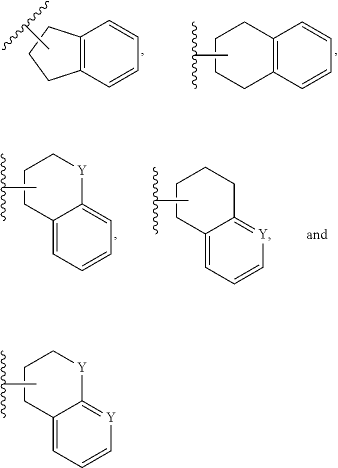

wherein each Y is independently selected from the group consisting of: —O—, —NR¹⁴— and —C(R²¹)_{q}— (wherein q is 0, 1 or 2 and each R²¹ is independently selected), and wherein R²¹ is as defined for Formula I and R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶).

In one embodiment of this invention R⁶ is H.

In another embodiment of this invention R⁶ is alkyl.

In another embodiment of this invention R⁶ is a C₁ to C₃ alkyl group.

In another embodiment of this invention R⁶ is methyl.

In another embodiment of this invention R⁶ is ethyl.

In another embodiment of this invention R⁶ is a C₃ alkyl group.

In another embodiment of this invention R⁶ is isopropyl.

In another embodiment R⁶ is —C(O)OR¹⁵.

In another embodiment R⁶ is - —C(O)OR¹⁵ wherein R¹⁵ is alkyl.

In another embodiment R⁶ is - —C(O)OR¹⁵ wherein R¹⁵ is methyl.

In another embodiment R⁶ is alkyl substituted with 1-5 R²¹ groups.

In another embodiment R⁶ is alkyl substituted with one R²¹ group.

In another embodiment R⁶ is alkyl substituted with one R²¹ group, and said R²¹ group is —OR¹⁵.

In another embodiment R⁶ is alkyl substituted with one R²¹ group, and said R²¹ group is —OR¹⁵, and said R¹⁵ is alkyl.

In another embodiment R⁶ is alkyl substituted with one R²¹ group, and said R²¹ group is —OR¹⁵, and said R¹⁵ is methyl.

In another embodiment R⁶ is —CH₂R²¹ (i.e. alkyl substituted with one R²¹ group, wherein said alkyl is —CH₂—).

In another embodiment R⁶ is —CH₂OR¹⁵ (i.e. alkyl substituted with one R²¹ group, wherein said alkyl is —CH₂—, and said R²¹ group is —OR¹⁵).

In another embodiment R⁶ is —CH₂OR¹⁵ (i.e. alkyl substituted with one R²¹ group, wherein said alkyl is —CH₂—, and said R²¹ group is —OR¹⁵), wherein said R¹⁵ group is alkyl.

In another embodiment R⁶ is —CH₂OR¹⁵ (i.e. alkyl substituted with one R²¹ group, wherein said alkyl is —CH₂—, and said R²¹ group is —OR¹⁵), wherein said R¹⁵ group is methyl.

In another embodiment R⁶ is —C(O)NR¹⁵R¹⁶.

In another embodiment R⁶ is —C(O)NR¹⁵R¹⁶ wherein R¹⁵ and R¹⁶ are each independently selected from the group consisting of: H and alkyl.

In another embodiment R⁶ is —C(O)NR¹⁵R¹⁶ wherein R¹⁵ and R¹⁶ are the same or different alkyl.

In another embodiment R⁶ is —C(O)NR¹⁵R¹⁶ wherein R¹⁵ and R¹⁶ are each independently selected from the group consisting of: H and methyl.

In another embodiment R⁶ is —C(O)NR¹⁵R¹⁶ wherein R¹⁵ and R¹⁶ are each methyl.

Other embodiments of this invention are directed to any one of the embodiments above directed to Ring (B) wherein:
(a) R⁶ is:
   (1) H, or
   (2) alkyl, or
   (3) methyl, or
   (4) ethyl, or
   (5) a C₃ alkyl group, or
   (6) isopropyl, or
   (7) —C(O)OR¹⁵, or
   (8) —C(O)OR¹⁵ wherein R¹⁵ is alkyl, or
   (9) —C(O)OR¹⁵ wherein R¹⁵ is methyl, or
   (10) alkyl substituted with 1-5 R²¹ groups, or
   (11) alkyl substituted with one R²¹ group, or
   (12) alkyl substituted with one R²¹ group, and said R²¹ group is —OR¹⁵, or
   (13) alkyl substituted with one R²¹ group, and said R²¹ group is —OR¹⁵, and said R¹⁵ is alkyl, or
   (14) alkyl substituted with one R²¹ group, and said R²¹ group is —OR¹⁵, and said R¹⁵ is methyl, or
   (15) —CH₂R²¹ (i.e. alkyl substituted with one R²¹ group, wherein said alkyl is —CH₂—), or
   (16) —CH₂OR¹⁵ (i.e. alkyl substituted with one R²¹ group, wherein said alkyl is —CH₂—, and said R²¹ group is —OR¹⁵), or
   (17) —CH₂OR¹⁵ (i.e. alkyl substituted with one R²¹ group, wherein said alkyl is —CH₂—, and said R²¹ group is —OR¹⁵), wherein said R¹⁵ group is alkyl, or
   (18) —CH₂OR¹⁵ (i.e. alkyl substituted with one R²¹ group, wherein said alkyl is —CH₂—, and said R²¹ group is —OR¹⁵), wherein said R¹⁵ group is methyl, or
   (19) —C(O)NR¹⁵R¹⁶, or
   (20) —C(O)NR¹⁵R¹⁶ wherein R¹⁵ and R¹⁶ are each independently selected from the group consisting of: H and alkyl, or
   (21) —C(O)NR¹⁵R¹⁶ wherein R¹⁵ and R¹⁶ are the same or different alkyl, or
   (22) —C(O)NR¹⁵R¹⁶ wherein R¹⁵ and R¹⁶ are each independently selected from the group consisting of: H and methyl, or
   (23) —C(O)NR¹⁵R¹⁶ wherein R¹⁵ and R¹⁶ are each methyl; and
(b) R¹ is as defined in any one of the embodiments above that are directed to R¹.

Other embodiments of this invention are directed to any one of the embodiments above directed to Ring (B) wherein:
(a) R⁶ is:
   (1) H, or
   (2) alkyl, or
   (3) methyl, or
   (4) ethyl, or
   (5) a C₃ alkyl group, or
   (6) isopropyl, or
   (7) —C(O)OR¹⁵, or
   (8) —C(O)OR¹⁵ wherein R¹⁵ is alkyl, or
   (9) —C(O)OR¹⁵ wherein R¹⁵ is methyl, or
   (10) alkyl substituted with 1-5 R²¹ groups, or
   (11) alkyl substituted with one R²¹ group, or
   (12) alkyl substituted with one R²¹ group, and said R²¹ group is —OR¹⁵, or
   (13) alkyl substituted with one R²¹ group, and said R²¹ group is —OR¹⁵, and said R¹⁵ is alkyl, or
   (14) alkyl substituted with one R²¹ group, and said R²¹ group is —OR¹⁵, and said R¹⁵ is methyl, or
   (15) —CH₂R²¹ (i.e. alkyl substituted with one R²¹ group, wherein said alkyl is —CH₂—), or
   (16) —CH₂OR⁵ (i.e. alkyl substituted with one R²¹ group, wherein said alkyl is —CH₂—, and said R²¹ group is —OR¹⁵), or
   (17) —CH₂OR¹⁵ (i.e. alkyl substituted with one R²¹ group, wherein said alkyl is —CH₂—, and said R²¹ group is —OR¹⁵), wherein said R¹⁵ group is alkyl, or
   (18) —CH₂OR¹⁵ (i.e. alkyl substituted with one R²¹ group, wherein said alkyl is —CH₂—, and said R²¹ group is —OR¹⁵), wherein said R¹⁵ group is methyl, or
   (19) —C(O)NR¹⁵R¹⁶, or
   (20) —C(O)NR¹⁵R¹⁶ wherein R¹⁵ and R¹⁶ are each independently selected from the group consisting of: H and alkyl, or
   (21) —C(O)NR¹⁵R¹⁶ wherein R¹⁵ and R¹⁶ are the same or different alkyl, or
   (22) —C(O)NR¹⁵R¹⁶ wherein R¹⁵ and R¹⁶ are each independently selected from the group consisting of: H and methyl, or
   (23) —C(O)NR¹⁵R¹⁶ wherein R¹⁵ and R¹⁶ are each methyl; and
(b) R¹ is selected from the group consisting of:

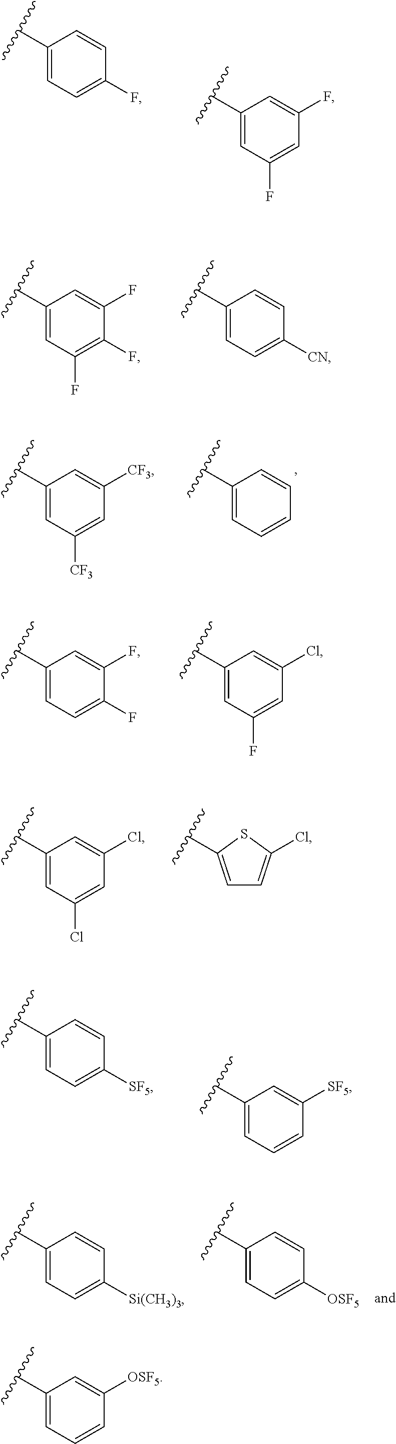

Other embodiments of this invention are directed to any one of the embodiments above directed to Ring (B) wherein:
(a) R⁶ is:
   (1) H, or
   (2) alkyl, or
   (3) methyl, or
   (4) ethyl, or
   (5) a C₃ alkyl group, or
   (6) isopropyl, or
   (7) —C(O)OR^{is}, or
   (8) —C(O)OR¹⁵ wherein R¹⁵ is alkyl, or
   (9) —C(O)OR¹⁵ wherein R¹⁵ is methyl, or
   (10) alkyl substituted with 1-5 R²¹ groups, or
   (11) alkyl substituted with one R²¹ group, or
   (12) alkyl substituted with one R²¹ group, and said R²¹ group is —OR¹⁵, or
   (13) alkyl substituted with one R²¹ group, and said R²¹ group is —OR¹⁵, and said R¹⁵ is alkyl, or
   (14) alkyl substituted with one R²¹ group, and said R²¹ group is —OR¹⁵, and said R¹⁵ is methyl, or
   (15) —CH₂R²¹ (i.e. alkyl substituted with one R²¹ group, wherein said alkyl is —CH₂—), or
   (16) —CH₂OR¹⁵ (i.e. alkyl substituted with one R²¹ group, wherein said alkyl is —CH₂—, and said R²¹ group is —OR¹⁵), or
   (17) —CH₂OR¹⁵ (i.e. alkyl substituted with one R²¹ group, wherein said alkyl is —CH₂—, and said R²¹ group is —OR¹⁵), wherein said R¹⁵ group is alkyl, or
   (18) —CH₂OR¹⁵ (i.e. alkyl substituted with one R²¹ group, wherein said alkyl is —CH₂—, and said R²¹ group is —OR¹⁵), wherein said R¹⁵ group is methyl, or
   (19) —C(O)NR¹⁵R¹⁶, or
   (20) —C(O)NR¹⁵R¹⁶ wherein R¹⁵ and R¹⁶ are each independently selected from the group consisting of: H and alkyl, or
   (21) —C(O)NR¹⁵R¹⁶ wherein R¹⁵ and R¹⁶ are the same or different alkyl, or
   (22) —C(O)NR¹⁵R¹⁶ wherein R¹⁵ and R¹⁶ are each independently selected from the group consisting of: H and methyl, or
   (23) —C(O)NR¹⁵R¹⁶ wherein R¹⁵ and R¹⁶ are each methyl; and
(b) R¹ is selected from the group consisting of:

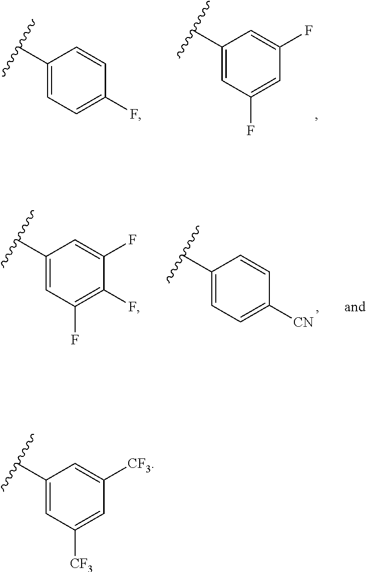

Other embodiments of this invention are directed to any one of the embodiments above directed to Ring (B) wherein R⁶ is H or alkyl and R¹ is as defined in any one of the above embodiments directed to R¹. In one such embodiment R⁶ is H or a C₁ to C₃ alkyl group. In another embodiment R⁶ is H or methyl. In another embodiment R⁶ is H or ethyl. In another embodiment R⁶ is H or a C₃ alkyl group. In another embodiment R⁶ is H or isopropyl.

The compounds of this invention are useful for treating central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

Thus, for example, the compounds of this invention can be used to treat the following diseases or conditions: Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), and Olfactory function loss (Getchell, et. al. Neurobiology of Aging, 663-673, 24, 2003).

In one embodiment of this invention U is CR²¹.

In another embodiment of this invention U is N.

In another embodiment of this invention U is C and the optional double bond is present.

In another embodiment of this invention U is CH.

In one embodiment of the invention:
(a) G is O and U is CR²¹,
(b) G is O and U is N,
(c) G is S and U is CR²¹,
(d) G is S and U is N,
(e) G is C(O) and U is CR²¹,
(f) G is C(O) and U is N,
(g) G is N(R¹⁴) and U is CR²¹, or
(h) G is G is N(R¹⁴) and U is N.

In one embodiment of this invention G¹ is a direct bond and ring A is a 5-membered ring.

In another embodiment of this invention G¹ is —O—.

In another embodiment of this invention G¹ is —C(R²¹)₂—.

In another embodiment of this invention G¹ is —CHR²¹—.

In another embodiment of this invention G¹ is —CR²¹— and the optional double bond to G¹ is present.

In another embodiment of this invention G¹ is —CH— and the optional double bond to G¹ is present.

In another embodiment of this invention G¹ is —N═ and the optional double bond to G¹ is present.

In another embodiment of this invention G¹ is —C(O)—.

In another embodiment of this invention G¹ is —C(═NR²)—.

In another embodiment of this invention G¹ is —S(O)₂—.

In another embodiment of this invention G¹ is —S(O)—.

In one embodiment of this invention G² is —O—.

In another embodiment of this invention G² is —C(R²¹)₂—.

In another embodiment of this invention G² is —CHR²¹—.

In another embodiment of this invention G² is —CR²¹— and the optional double bond to G² is present.

In another embodiment of this invention G² is —CH— and the optional double bond to G² is present.

In another embodiment of this invention G² is —CH₂—.

In another embodiment of this invention G² is —N═ and the optional double bond to G² is present.

In another embodiment of this invention G² is —C(O)—.

In another embodiment of this invention G² is —C(═NR²)—.

In another embodiment of this invention G² is —S(O)₂—.

In another embodiment of this invention G² is —S(O)—.

In one embodiment of this invention G³ is —C(R²¹)₂—.

In another embodiment of this invention G³ is —CH(R²¹)—.

In another embodiment of this invention G³ is —CH— and the optional double bond to G³ is present.

In another embodiment of this invention G³ is —CR²¹— and the optional double bond to G³ is present.

In another embodiment of this invention G³ is —CH₂—.

In another embodiment of this invention G³ is —N═ and the optional double bond to G³ is present.

In one embodiment of this invention W is —O—.

In another embodiment of this invention W is —S—.

In another embodiment of this invention W is —C(R²¹)₂—.

In another embodiment of this invention W is —CHR²¹—.

In another embodiment of this invention W is —CH₂—.

In another embodiment of this invention W is —C(O)—.

In another embodiment of this invention W is —C(═NR²)—.

In another embodiment of this invention W is —S(O)₂—.

In another embodiment of this invention W is —S(O)—.

In one embodiment of this invention V is a direct bond and ring B is a 5-membered ring.

In another embodiment of this invention V is —O—.

In another embodiment of this invention V is —C(R²¹)₂—.

In another embodiment of this invention V is —CHR²¹—.

In another embodiment of this invention V is —CH₂—.

In another embodiment of this invention V is —NR²—.

In another embodiment of this invention V is —C(O)—.

In another embodiment of this invention V is —C(═NR²)—.

In another embodiment of this invention V is —S(O)₂—.

In another embodiment of this invention V is —S(O)—.

In one embodiment of this invention G is O.

In another embodiment of this invention G is O and U is N.

In another embodiment of this invention G is S.

In another embodiment of this invention G is S and U is N.

In one embodiment of this invention G is C(O).

In another embodiment of this invention G is C(O) and U is N.

In another embodiment of this invention G is C(O).

In another embodiment of this invention G is C(O) and U is N.

In one embodiment of this invention G is N(R²).

In another embodiment of this invention G is N(R²) and U is N.

In one embodiment of this invention G is N(R²), and R² is H (i.e. G is NH).

In another embodiment of this invention G is N(R²), R² is H (i.e. G is NH), and U is N.

Other embodiments of this invention are directed to any one of the above embodiments wherein G is N(R²), wherein R² is H (i.e. G is NH).

In one embodiment R² (of the NR² moiety) is H.

In another embodiment R² (of the NR² moiety) is alkyl, such as, for example, methyl, ethyl or isopropyl.

In another embodiment R² (of the NR² moiety) is aryl, such as, for example, phenyl.

In another embodiment R² (of the NR² moiety) is substituted aryl, such as, for example, substituted phenyl.

In another embodiment R² (of the NR² moiety) is —C(O)R⁴ wherein R⁴ is alkyl (such as, for example, methyl, ethyl or isopropyl).

In another embodiment R² (of the NR² moiety) is —C(O)R⁴ wherein R⁴ is aryl, such as, for example, phenyl.

In another embodiment R² (of the NR² moiety) is —C(O)R⁴ wherein R⁴ is substituted aryl, such as, for example, substituted phenyl.

In another embodiment of this invention R⁵ is H.

In another embodiment of this invention, the cycloalkyl G moiety is unsubstituted.

In another embodiment of this invention, the cycloalkyl G moiety is substituted with 1 to 6 independently selected R²¹ groups.

In another embodiment of this invention, the cycloalkyl G moiety is a C₃ to C₁₀ cycloalkyl substituted with 1 to 6 independently selected R²¹ groups. In one example G is a cyclobutanone ring.

In one embodiment of this invention, the cycloalkyl G moiety is a C₃ to C₁₀ cycloalkyl. In one example, said cycloalkyl is selected from the group consisting of: cyclopropyl, cyclobutyl, cyclopentyl and cyclohexyl. In another example of said cycloalkyl G moiety the cycloalkyl ring carbon by which said cycloalkyl moiety is bound to ring A is different from the cycloalkyl ring carbon by which said cycloalkyl moiety is bound to moiety R¹⁰. In another example of said cycloalkyl G moiety the cycloalkyl ring is bound to ring A and the R¹⁰ moiety by the same cycloalkyl ring carbon.

In another embodiment of this invention, the heterocycloalkyl G moiety is unsubstituted.

In another embodiment of this invention, the heterocycloalkyl G moiety is unsubstituted and said heterocycloalkyl G moiety comprises 1 to 4 heteroatoms independently selected from the group consisting of: —O—, —NR²—, —S—, —S(O)—, and —S(O)₂.

In another embodiment of this invention, the heterocycloalkyl G moiety is substituted with 1 to 6 independently selected R²¹ groups, and said heterocycloalkyl G moiety comprises 1 to 4 ring heteroatoms independently selected from the group consisting of: —O—, —NR²—, —S—, —S(O)—, and —S(O)₂.

In one embodiment of this invention, the heterocycloalkyl G moiety comprises 1 to 4 heteroatoms. In one example, said heterocycloalkyl G moiety comprises 1 to 4 heteroatoms. In another example, said heterocycloalkyl G moiety comprises 1 to 3 heteroatoms. In another example, said heterocycloalkyl G moiety comprises 1 to 2 heteroatoms. In another example, said heterocycloalkyl G moiety comprises 1 heteroatom. The heteroatoms in said heterocycloalkyl G moiety are independently selected from the group consisting of —O—, —NR²—, —S—, —S(O)—, and —S(O)₂. In one example, said heterocycloalkyl G moiety is bound to the R¹⁰ moiety and to ring A by the same heterocycloalkyl ring atom. In another example, said heterocycloalkyl moiety is bound to the R¹⁰ moiety and to ring A by different heterocycloalkyl ring atoms, and wherein the heterocycloalkyl ring atoms that bind the heterocycloalkyl moiety to R¹⁰ and to ring A are selected from the group consisting of carbon and nitrogen.

An example of said alkynyl G moiety is:

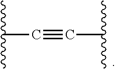

In another embodiment of this invention G is selected from the group consisting of: a direct bond, cycloalkyl (e.g., C₃ to C₁₀, and also for example, cyclopropyl, cyclobutyl, cyclopentyl and cyclohexyl, (wherein in one example the cycloalkyl ring carbon by which said cycloalkyl moiety is bound to ring A is different from the cycloalkyl ring carbon by which said cycloalkyl moiety is bound to moiety R¹⁰, and wherein in another example said cycloalkyl ring is bound to ring A and the R¹⁰ moiety by the same cycloalkyl ring carbon)), heterocycloalkyl (wherein said heterocycloalkyl comprises 1 to 4 heteroatoms, and in one example, 1 to 4 heteroatoms, and in another example 1 to 3 heteroatoms, and in another example 1 to 2 heteroatoms, and in another example 1 heteroatom, and wherein said heteroatoms are selected from the group consisting of —O—, —NR²—, —S—, —S(O)—, and —S(O)₂, and wherein in one example said heterocycloalkyl moiety is bound to the R¹⁰ moiety and ring A by the same heterocycloalkyl ring atom, and in another example said heterocycloalkyl moiety is bound to the R¹⁰ moiety and ring A by different heterocycloalkyl ring atoms, and wherein the heterocycloalkyl ring atoms that bind the heterocycloalkyl moiety to R¹⁰ and ring A are selected from the group consisting of carbon and nitrogen), —C═C—, —CF₂—, alkynyl (e.g., —C≡C—), —NH—, —N(R²)— (and in one example, —NH—), —O—, —CR⁴(OH)—, —CR⁴(OR⁴)—, —(CH₂)ᵣN(R²)—, —N(R²)(CH₂)ᵣ—, —(CH₂)₂₋₃—, —(C(R⁴)₂)ᵣ— (wherein each R⁴ is independently selected), —(CHR⁴)₂₋₃— (wherein each R⁴ is independently selected), —S—, —S(O)—, and —S(O)₂.

In another embodiment of this invention t is 1.

In another embodiment of this invention t is 2.

In another embodiment of this invention r is 1.

In another embodiment of this invention r is 2.

In another embodiment of this invention r is 3.

In another embodiment of this invention G is selected from the group consisting of: a direct bond, and —N(R²)— (e.g., —NH—).

In another embodiment of this invention G is —N(R²)— (e.g., —NH—).

In another embodiment of this invention G is a cycloalkyl.

In another embodiment of this invention G is a heterocycloalkyl.

In another embodiment of this invention G is —C═C—.

In another embodiment of this invention G is —CF₂—.

In another embodiment of this invention G is alkynyl.

In another embodiment of this invention G is —O—.

In another embodiment of this invention G is —CR⁴(OH)—.

In another embodiment of this invention G is —CR⁴(OR⁴)—.

In another embodiment of this invention G is —(CH₂)ᵣN(R²)—.

In another embodiment of this invention G is —N(R²)(CH₂)ᵣ—.

In another embodiment of this invention G is —(CH₂)₂₋₅—.

In another embodiment of this invention G is —(C(R⁴)₂)ᵣ— (wherein each R⁴ is independently selected).

In another embodiment of this invention G is —(CHR⁴)₂₋₅— (wherein each R⁴ is independently selected).

In another embodiment of this invention G is —S—.

In another embodiment of this invention G is —S(O)—.

In another embodiment of this invention G is —S(O)₂.

In another embodiment of this invention G is —C(O)—.

In another embodiment of this invention G is —(C═NR²)—.

In another embodiment of this invention G is —(C═C(R⁶)₂)—.

In another embodiment of this invention G is —(CHR³)—.

In another embodiment of this invention ring (B) is not substituted with any R²¹ groups.

In another embodiment of this invention, there are 1 to 5 R²¹ groups present in Formula I, and at least one (e.g., 1 to 2) R²¹ is selected from the group consisting of: —SF₅, —OSF₅ and —Si(R^{15A})₃, wherein each R^{15A} is independently selected.

In another embodiment of this invention, there are 1 to 5 R²¹ groups present in Formula I, and at least one R²¹ is selected from the group consisting of: —SF₅ and —Si(R^{15A})₃, and each R^{15A} is the same or different alkyl group.

In another embodiment of this invention, there are 1 to 5 R²¹ groups present in Formula I, and at least one R²¹ is selected from the group consisting of: —SF₅, —OSF₅ and —Si(CH₃)₃.

In another embodiment of this invention, there are 1 to 5 R²¹ groups present in Formula I, and one of the R²¹ groups is selected from the group consisting of: —SF₅, OSF₅ and —Si(R^{15A})₃.

In another embodiment of this invention, there are 1 to 5 R²¹ groups present in Formula I, and one of the R²¹ groups is selected from the group consisting of: —SF₅, OSF₅ and —Si(R^{15A})₃, and each R^{15A} is the same or different alkyl group.

In another embodiment of this invention, there are 1 to 5 R²¹ groups present in Formula I, and one of the R²¹ groups is selected from the group consisting of: —SF₅, —OSF₅ and —Si(CH₃)₃.

In another embodiment of this invention, there are 2 to 5 R²¹ groups present in Formula I, and two of the R²¹ groups are selected from the group consisting of: —SF₅, OSF₅ and —Si(R^{15A})₃, wherein each R^{15A} is independently selected.

In another embodiment of this invention, there are 2 to 5 R²¹ groups present in Formula I, and two of the R²¹ groups are selected from the group consisting of: —SF₅, OSF₅ and —Si(R^{15A})₃, and each R^{15A} is the same or different alkyl group.

In another embodiment of this invention, there are 2 to 5 R²¹ groups present in Formula I, and two of the R²¹ groups are selected from the group consisting of: —SF₅, —OSF₅ and —Si(CH₃)₃.

In another embodiment of this invention, there are 1 to 5 R²¹ groups present in Formula I, and at least one (e.g., 1 to 2) R²¹ is selected from the group consisting of: —SF₅ and —Si(R^{15A})₃, wherein each R^{15A} is independently selected.

In another embodiment of this invention, there are 1 to 5 R²¹ groups present in Formula I, and at least one R²¹ is selected from the group consisting of: —SF₅ and —Si(R^{15A})₃, and each R^{15A} is the same or different alkyl group.

In another embodiment of this invention, there are 1 to 5 R²¹ groups present in Formula I, and at least one R²¹ is selected from the group consisting of: —SF₅ and —Si(CH₃)₃.

In another embodiment of this invention, there are 1 to 5 R²¹ groups present in Formula I, and one of the R²¹ groups is selected from the group consisting of: —SF₅ and —Si(R^{15A})₃.

In another embodiment of this invention, there are 1 to 5 R²¹ groups present in Formula I, and one of the R²¹ groups is selected from the group consisting of: —SF₅ and —Si(R^{15A})₃, and each R^{15A} is the same or different alkyl group.

In another embodiment of this invention, there are 1 to 5 R²¹ groups present in Formula I, and one of the R²¹ groups is selected from the group consisting of: —SF₅ and —Si(CH₃)₃.

In another embodiment of this invention, there are 2 to 5 R²¹ groups present in Formula I, and two of the R²¹ groups are selected from the group consisting of: —SF₅ and —Si(R^{15A})₃, wherein each R^{15A} is independently selected.

In another embodiment of this invention, there are 2 to 5 R²¹ groups present in Formula I, and two of the R²¹ groups are selected from the group consisting of: —SF₅ and —Si(R^{15A})₃, and each R^{15A} is the same or different alkyl group.

In another embodiment of this invention, there are 2 to 5 R²¹ groups present in Formula I, and two of the R²¹ groups are selected from the group consisting of: —SF₅ and —Si(CH₃)₃.

In another embodiment of this invention, there are 1 to 5 R²¹ groups present in Formula I, and one of the R²¹ groups is —SF₅.

In another embodiment of this invention, there are 2 to 5 R²¹ groups present in Formula I, and two of the R²¹ groups are —SF₅.

In another embodiment of this invention, there are 1 to 5 R²¹ groups present in Formula I, and one of the R²¹ groups is —OSF₅.

In another embodiment of this invention, there are 2 to 5 R²¹ groups present in Formula I, and two of the R²¹ groups are —OSF₅.

In another embodiment of this invention, there are 1 to 5 R²¹ groups present in Formula I, and one of the R²¹ groups is —Si(R^{15A})₃.

In another embodiment of this invention, there are 1 to 5 R²¹ groups present in Formula I, and one of the R²¹ groups is —Si(R^{15A})₃ and each R^{15A} is the same or different alkyl group.

In another embodiment of this invention, there are 1 to 5 R²¹ groups present in Formula I, and one of the R²¹ groups is —Si(CH₃)₃.

In another embodiment of this invention, there are 2 to 5 R²¹ groups present in Formula I, and two of the R²¹ groups are the same or different —Si(R^{15A})₃, wherein each R^{15A} is independently selected.

In another embodiment of this invention, there are 2 to 5 R²¹ groups present in Formula I, and two of the R²¹ groups are the same or different —Si(R^{15A})₃ and each R^{15A} is the same or different alkyl group.

In another embodiment of this invention, there are 2 to 5 R²¹ groups present in Formula I, and two of the R²¹ groups are —Si(CH₃)₃.

In one embodiment of this invention R¹ is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, —and heterocyclyalkyl-; wherein each of said alkyl-, alkenyl-, alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups.

In another embodiment of this invention R¹ is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, —and heterocyclyalkyl-; wherein: (a) each of said alkyl-, alkenyl-alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl and heterocyclyalkyl-R¹ groups is optionally substituted with 1-5 independently selected R²¹ groups; and (b) each of said fused benzocycloalkyl, fused benzoheterocycloalkyl, fused heteroarylcycloalkyl, and fused heteroarylheterocycloalkyl R¹ groups is optionally substituted with 1-5 independently selected R²¹ groups.

In another embodiment of this invention, R¹ is selected from the group consisting of: alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl, —and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl and heterocyclyalkyl-R¹ groups is optionally substituted with 1-5 independently selected R²¹ groups.

In another embodiment of this invention, R¹ is selected from the group consisting of: fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), and fused heteroarylheterocycloalkyl (i.e., heteroarylfusedheterocycloalkyl); wherein each of said fused benzocycloalkyl, fused benzoheterocycloalkyl, fused heteroarylcycloalkyl, and fused heteroarylheterocycloalkyl R¹ groups is optionally substituted with 1-5 independently selected R²¹ groups.

In another embodiment of this invention R¹ is substituted with R²¹ groups, and at least one (e.g. 1 to 2) of the R²¹ groups is selected from the group consisting of: —SF₅, —OSF₅ and —Si(R^{15A})₃, wherein each R^{15A} is independently selected.

In another embodiment of this invention R¹ is substituted with R²¹ groups, and at least one (e.g. 1 to 2) of the R²¹ groups is selected from the group consisting of: —SF₅, —OSF₅ and —Si(R^{15A})₃, and each R^{15A} is the same or different alkyl group.

In another embodiment of this invention R¹ is substituted with R²¹ groups, and at least one (e.g. 1 to 2) of the R²¹ groups is selected from the group consisting of: —SF₅, —OSF₅ and —Si(CH₃)₃.

In another embodiment of this invention R¹ is substituted with R²¹ groups, and one R²¹ group is selected from the group consisting of: —SF₅, —OSF₅ and —Si(R^{15A})₃, wherein each R^{15A} is independently selected.

In another embodiment of this invention R¹ is substituted with R²¹ groups, and one R²¹ group is selected from the group consisting of: —SF₅, —OSF₅ and —Si(R^{15A})₃, and each R^{15A} is the same or different alkyl group.

In another embodiment of this invention R¹ is substituted with R²¹ groups, and one R²¹ group is selected from the group consisting of: —SF₅, —OSF₅ and —Si(CH₃)₃.

In another embodiment of this invention R¹ is substituted with R²¹ groups, and two R²¹ groups are selected from the group consisting of: —SF₅, —OSF₅ and —Si(R^{15A})₃, wherein each R^{15A} is independently selected.

In another embodiment of this invention R¹ is substituted with R²¹ groups, and two R²¹ groups are selected from the group consisting of: —SF₅, —OSF₅ and —Si(R^{15A})₃, and each R^{15A} is the same or different alkyl group.

In another embodiment of this invention R¹ is substituted with R²¹ groups, and two R²¹ groups are selected from the group consisting of: —SF₅, —OSF₅ and —Si(CH₃)₃.

In another embodiment of this invention R¹ is substituted with R²¹ groups, and one R²¹ group is —SF₅.

In another embodiment of this invention R¹ is substituted with R²¹ groups, and two R²¹ groups are —SF₅.

In another embodiment of this invention R¹ is substituted with R²¹ groups, and one R²¹ group is —OSF₅.

In another embodiment of this invention R¹ is substituted with R²¹ groups, and two R²¹ groups are —OSF₅.

In another embodiment of this invention R¹ is substituted with R²¹ groups, and one R²¹ group is —Si(R^{15A})₃, wherein each R^{15A} is independently selected.

In another embodiment of this invention R¹ is substituted with R²¹ groups, and one R²¹ group is —Si(R^{15A})₃ and each R^{15A} is the same or different alkyl group.

In another embodiment of this invention R¹ is substituted with R²¹ groups, and one R²¹ group is —Si(CH₃)₃.

In another embodiment of this invention R¹ is substituted with R²¹ groups, and two of the R²¹ groups are the same or different —Si(R^{15A})₃, wherein each R^{15A} is independently selected.

In another embodiment of this invention R¹ is substituted with R²¹ groups, and two of the R²¹ groups are the same or different —Si(R^{15A})₃ group, and each R^{15A} is the same or different alkyl group.

In another embodiment of this invention R¹ is substituted with R²¹ groups, and two of the R²¹ group are —Si(CH₃)₃.

In another embodiment of this invention R¹ is an aryl group substituted with R²¹ groups, and at least one (e.g., 1 to 2) R²¹ group is selected from the group consisting of: —SF₅, —OSF₅ and —Si(R^{15A})₃, wherein each R^{15A} is independently selected.

In another embodiment of this invention R¹ is an aryl group substituted with R²¹ groups, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and at least one (e.g., 1 to 2) R²¹ group is selected from the group consisting of: —SF₅, —OSF₅ and —Si(CH₃)₃.

In another embodiment of this invention R¹ is an aryl group substituted with R²¹ groups, and said aryl moiety is phenyl, and at least one (e.g., 1 to 2) R²¹ group is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and at least one (e.g., 1 to 2) R²¹ group is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —SF₅.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R¹ group on said phenyl is —OSF₅.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, wherein each R^{15A} is independently selected.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(CH₃)₃.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —SFₛ.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —OSF₅.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, wherein each R^{15A} is independently selected.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —Si(CH₃)₃.

In another embodiment of this invention R¹ is an alkyl group substituted with one or more independently selected R²¹ groups.

In another embodiment of this invention R¹ is:

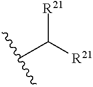

wherein each R²¹ is independently selected, and each R²¹ is independently unsubstituted or substituted with one or more independently selected R²² groups.

In another embodiment of this invention R¹ is:

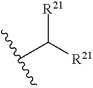

wherein one R²¹ is an unsubstituted or substituted alkyl group.

In another embodiment of this invention R¹ is:

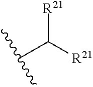

wherein one R²¹ is an unsubstituted alkyl group.

In another embodiment of this invention R¹ is:

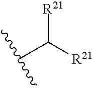

wherein one R²¹ is a substituted alkyl group.

In another embodiment of this invention R¹ is:

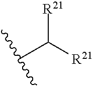

wherein one R²¹ is an unsubstituted or substituted alkyl group, and the other R²¹ is an unsubstituted or substituted aryl (e.g., phenyl) group.

In another embodiment of this invention R¹ is:

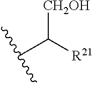

and R²¹ is unsubstituted or substituted with one or more independently selected R²² groups.

In another embodiment of this invention R¹ is:

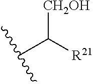

and R²¹ is unsubstituted aryl (e.g., phenyl) or aryl (e.g., phenyl) substituted with one or more independently selected R²² groups.

In another embodiment of this invention R¹ is an alkyl group substituted with one R²¹ group.

In another embodiment of this invention R¹ is an alkyl group substituted with one R²¹ group, and said R²¹ group is substituted with one or more independently selected R²² groups.

In another embodiment of this invention R¹ is:

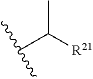

wherein R²¹ is unsubstituted or substituted with one or more independently selected R²² groups.

In another embodiment of this invention R¹ is an alkyl group substituted with one R²¹ group, and said R²¹ group is an aryl group.

In another embodiment of this invention R¹ is an alkyl group substituted with one R²¹ group, and said R²¹ group is an aryl group, said aryl is phenyl.

In another embodiment of this invention R¹ is an ethyl group substituted with one R²¹ group, and said R²¹ group is an aryl group, said aryl is phenyl.

In another embodiment of this invention R¹ is a methyl group substituted with one R²¹ group, and said R²¹ group is an aryl group, said aryl is phenyl.

In another embodiment of this invention R¹ is an alkyl group substituted with one R²¹ group, and said R²¹ group is an aryl group, and said aryl group is substituted with one or more R²² groups.

In another embodiment of this invention R¹ is an alkyl group substituted with one R²¹ group, and said R²¹ group is an aryl group, and said aryl group is substituted with one or more R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is an aryl group, and said aryl group is substituted with one or two R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is an aryl group, and said aryl group is substituted with one R²² group, and said R²² group is selected from the group consisting of: —SF₅, —OSF₅, —Si(R^{15A})₃.

In another embodiment of this invention R¹ is an alkyl group substituted with one R²¹ group, and said R²¹ group is an aryl group, and said aryl group is substituted with one or more R²² groups, and each R²² group is the same or different halo.

In another embodiment of this invention R¹ is an alkyl group substituted with one R²¹ group, and said R²¹ group is an aryl group, and said aryl group is substituted with 1 to 3 R²² groups, and each R²² group is the same or different halo.

In another embodiment of this invention R¹ is an alkyl group substituted with one R²¹ group, and said R²¹ group is an aryl group, and said aryl group is substituted with one or two R²² halo groups, and each R²² group is the same or different halo.

In another embodiment of this invention R¹ is an alkyl group substituted with one R²¹ group, and said R²¹ group is an aryl group, and said aryl group is substituted with one or two R²²F groups.

In another embodiment of this invention R¹ is an alkyl group substituted with one R²¹ group, and said R²¹ group is phenyl, and said phenyl is substituted with one or more R²² groups.

In another embodiment of this invention R¹ is an alkyl group substituted with one R²¹ group, and said R²¹ group is phenyl, and said phenyl is substituted with one more R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is phenyl, and said phenyl is substituted with one or two R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is phenyl, and said phenyl is substituted with one

R²² group, and said R²² group is selected from the group consisting of: —SF₅, —OSF₅, —Si(R^{15A})₃.

In another embodiment of this invention R¹ is an alkyl group substituted with one R²¹ group, and said R²¹ group is phenyl, and said phenyl is substituted with one or more R²² groups, and each R²² group is the same or different halo.

In another embodiment of this invention R¹ is an alkyl group substituted with one R²¹ group, and said R²¹ group is phenyl, and said phenyl is substituted with one or two R²² halo groups, and each R²² group is the same or different halo.

In another embodiment of this invention R¹ is an alkyl group substituted with one R²¹ group, and said R²¹ group is phenyl, and said phenyl is substituted with one or two R²²F groups.

In another embodiment of this invention R¹ is an ethyl group substituted with one R²¹ group, and said R²¹ group is an aryl group, and said aryl group is substituted with one or more R²² groups.

In another embodiment of this invention R¹ is a methyl group substituted with one R²¹ group, and said R²¹ group is an aryl group, and said aryl group is substituted with one or more R²² groups.

In another embodiment of this invention R¹ is an ethyl group substituted with one R²¹ group, and said R²¹ group is phenyl, and said phenyl is substituted with one or more R²² groups.

In another embodiment of this invention R¹ is an ethyl group substituted with one R²¹ group, and said R²¹ group is phenyl, and said phenyl is substituted with one or more R²² groups, and each R²² group is independently selected from the group consisting of: —SF₅, —OSF₅, —Si(R^{15A})₃.

In another embodiment of this invention R¹ is a methyl group substituted with one R²¹ group, and said R²¹ group is phenyl, and said phenyl is substituted with one or more R²² groups.

In another embodiment of this invention R¹ is an methyl group substituted with one R²¹ group, and said R²¹ group is phenyl, and said phenyl is substituted with one or more R²² groups, and each R²² group is independently selected from the group consisting of: —SF₅, —OSF₅, —Si(R^{15A})₃.

In another embodiment of this invention R¹ is an ethyl group substituted with one R²¹ group, and said R²¹ group is phenyl, and said phenyl is substituted with one or two R²² halo groups, and each R²² group is the same or different halo.

In another embodiment of this invention R¹ is a methyl group substituted with one R²¹ group, and said R²¹ group is phenyl, and said phenyl is substituted with one or two R²² halo groups, and each R²² group is the same or different halo.

In another embodiment of this invention R¹ is an ethyl group substituted with one R²¹ group, and said R²¹ group is phenyl, and said phenyl is substituted with one or two R²²F groups.

In another embodiment of this invention R¹ is a methyl group substituted with one R²¹ group, and said R²¹ group is phenyl, and said phenyl is substituted with one or two R²²F groups.

In another embodiment of this invention R¹ is an ethyl group substituted with one R²¹ group, and said R²¹ group is phenyl, and said phenyl is substituted with one R²² halo group.

In another embodiment of this invention R¹ is a methyl group substituted with one R²¹ group, and said R²¹ group is phenyl, and said phenyl is substituted with one R²² halo group.

In another embodiment of this invention R¹ is an ethyl group substituted with one R²¹ group, and said R²¹ group is phenyl, and said phenyl is substituted with one R²²F group.

In another embodiment of this invention R¹ is a methyl group substituted with one R²¹ group, and said R²¹ group is phenyl, and said phenyl is substituted with one R²²F group.

In another embodiment R¹ is selected from the group consisting of:

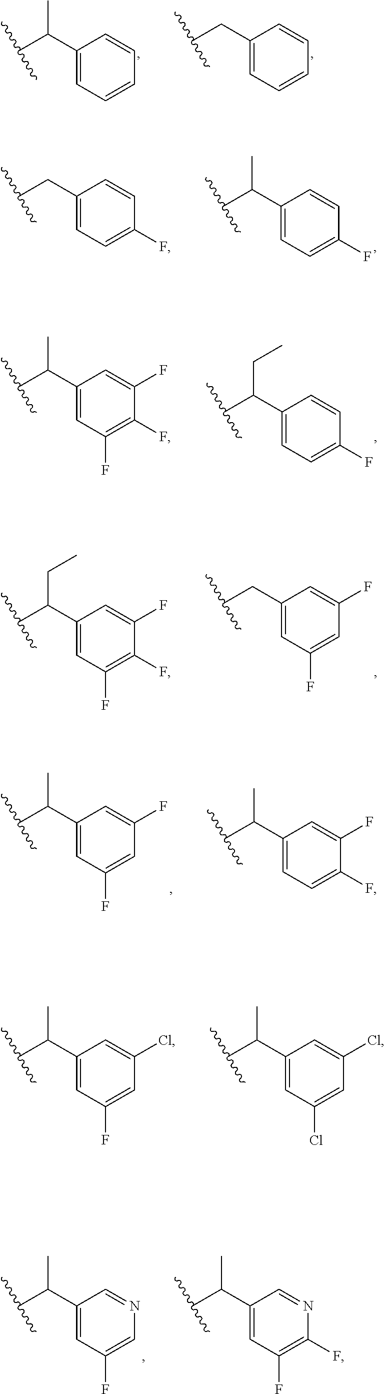

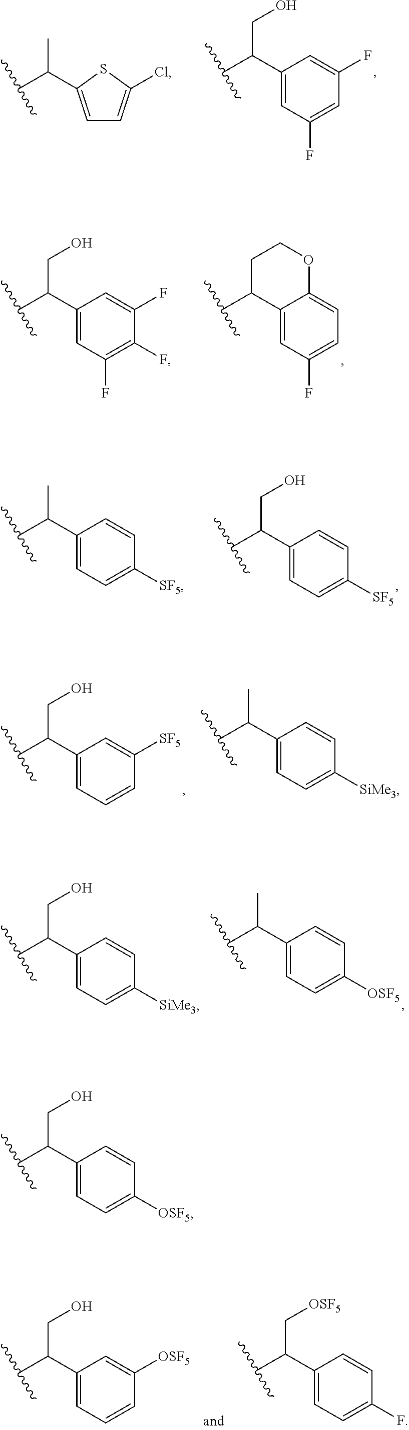

In another embodiment of this invention R¹ is selected from the group consisting of:

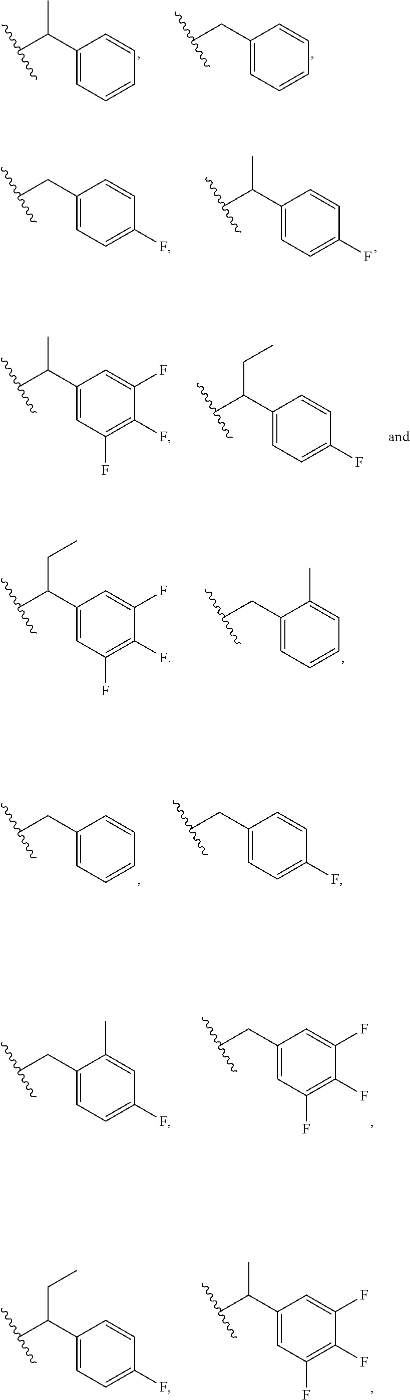

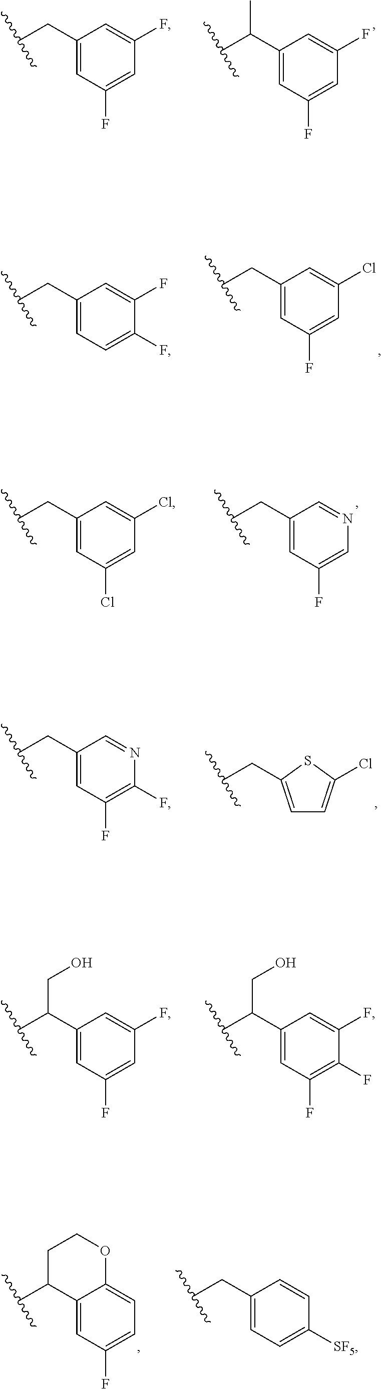

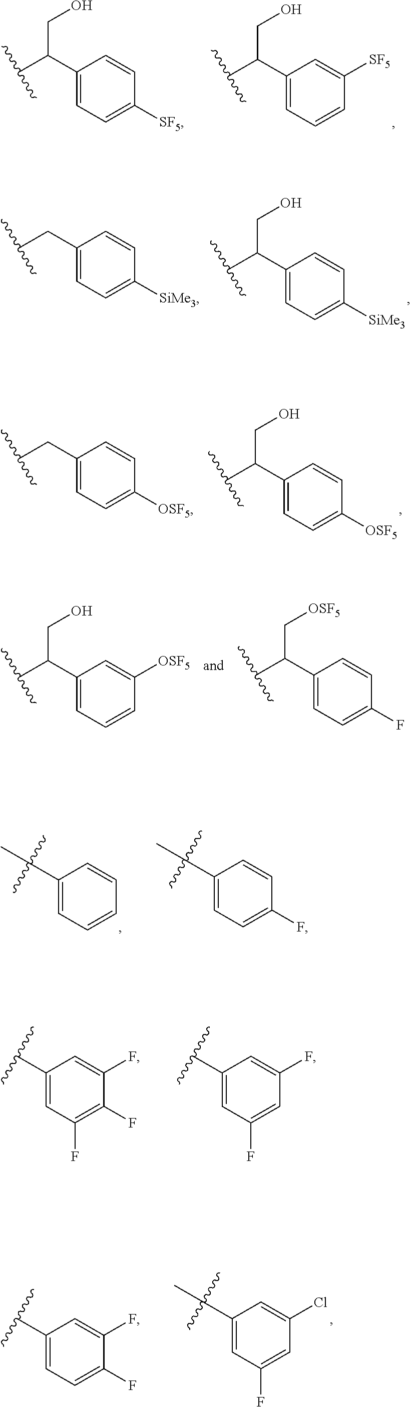

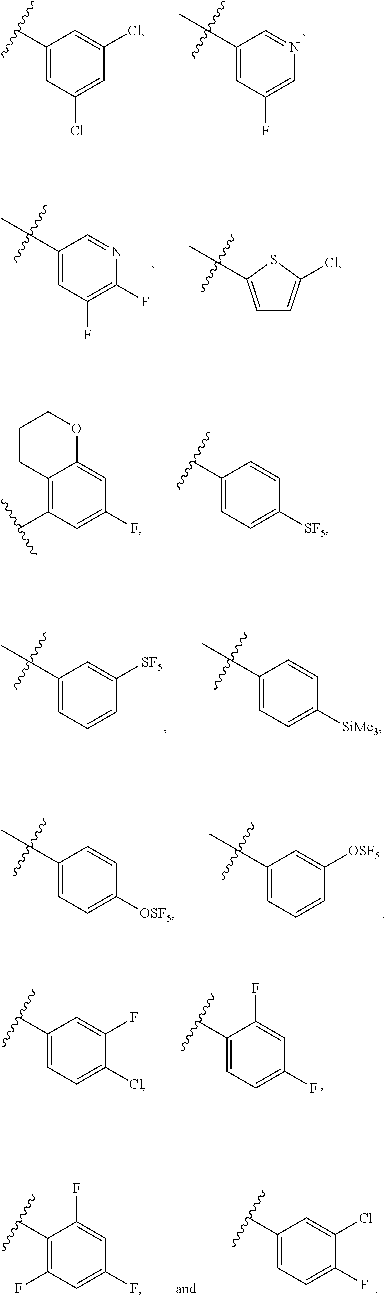

In another embodiment of this invention R¹ is an unsubstituted aryl group (e.g., an unsubstituted phenyl group). Thus, in another embodiment R¹ is phenyl.

In another embodiment of this invention R¹ is a substituted aryl group (e.g., a substituted phenyl group). Thus, in another embodiment R¹ is a substituted phenyl group.

In another embodiment of this invention R¹ is an aryl group substituted with 1 to 3 independently selected R²¹ groups.

In another embodiment of this invention R¹ is an aryl group substituted with one to 3 R²¹ groups, and each R²¹ group is the same or different halo.

In another embodiment of this invention R¹ is an aryl group substituted with one to 3 R²¹ groups, and each R²¹ group is F.

In another embodiment of this invention R¹ is phenyl, and said phenyl is substituted with one or more independently selected R²¹ groups.

In another embodiment of this invention R¹ is phenyl, and said phenyl is substituted with 1 to 3 independently selected R²¹ groups.

In another embodiment of this invention R¹ is phenyl, and said phenyl is substituted with 1 or 2 independently selected R²¹ groups.

In another embodiment of this invention R¹ is phenyl, and said phenyl is substituted with 1 R²¹ group.

In another embodiment of this invention R¹ is phenyl, and said phenyl is substituted with 1 to 3 R²¹ groups, and each R²¹ group is the same or different halo.

In another embodiment of this invention R¹ is phenyl, and said phenyl is substituted with three R²¹ halo groups, and each R²¹ group is the same or different halo.

In another embodiment of this invention R¹ is phenyl, and said phenyl is substituted with two R²¹ halo groups, and each R²¹ group is the same or different halo.

In another embodiment of this invention R¹ is phenyl, and said phenyl is substituted with one R²¹ halo group.

In another embodiment of this invention R¹ is phenyl, and said phenyl is substituted with 1 to 3 F (i.e., said phenyl is substituted with 1 to 3 R²¹ groups, and said R²¹ groups are halo, and said halo is F).

In another embodiment of this invention R¹ is phenyl, and said phenyl is substituted with one F (i.e., said phenyl is substituted with one R²¹ group, and said R²¹ group is halo, and said halo is F).

In another embodiment of this invention R¹ is phenyl, and said phenyl is substituted with two F atoms (i.e., said phenyl is substituted with two R²¹ groups, and said R²¹ groups are halo, and said halo is F).

In another embodiment of this invention R¹ is phenyl, and said phenyl is substituted with three F atoms (i.e., said phenyl is substituted with three R²¹ groups, and said R²¹ groups are halo, and said halo is F).

In another embodiment of this invention R¹ is phenyl, and said phenyl is substituted with one —CN group.

In another embodiment of this invention R¹ is phenyl, and said phenyl is substituted with one or two R²¹ alkyl groups (e.g. methyl groups), wherein each R²¹ group is substituted with 1 to 3 R²² halo groups (e.g. F groups).

In another embodiment of this invention R¹ is phenyl, and said phenyl is substituted with one or two —CF₃ groups (i.e. there are one or two R²¹ alkyl groups (i.e. methyl groups) each substituted with 3 R²² halo (i.e. F) groups).

In another embodiment of this invention R¹ is selected from the group consisting of:

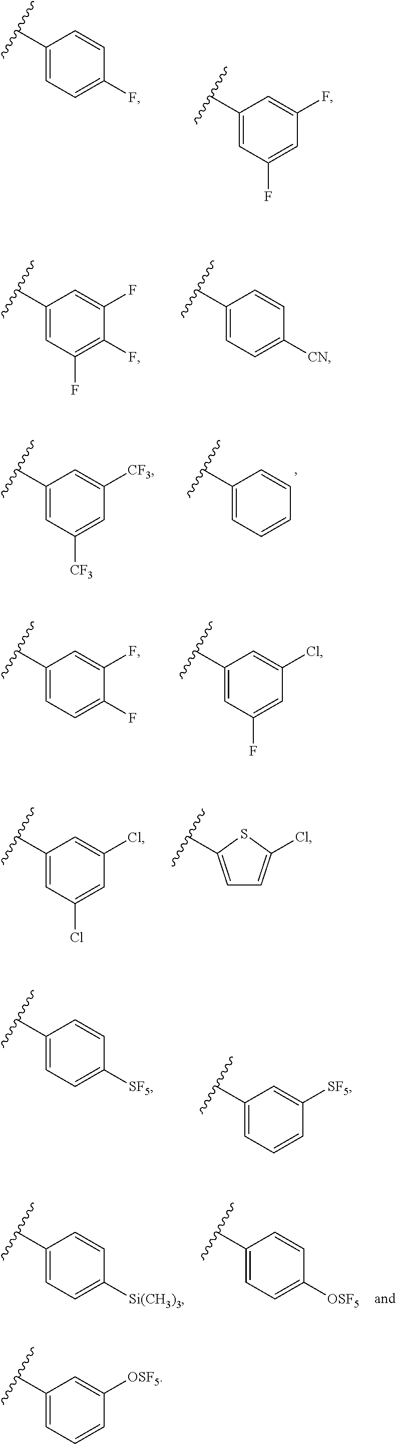

In another embodiment of this invention R¹ is:

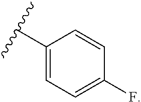

In another embodiment of this invention R¹ is:

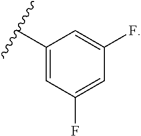

In another embodiment of this invention R¹ is:

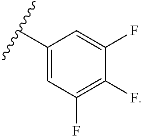

In another embodiment of this invention R¹ is:

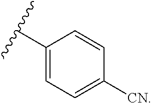

In another embodiment of this invention R¹ is:

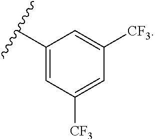

In another embodiment of this invention R¹ is:

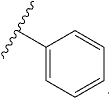

In another embodiment of this invention R¹ is:

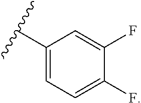

In another embodiment of this invention R¹ is:

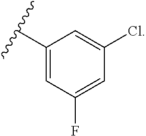

In another embodiment of this invention R¹ is:

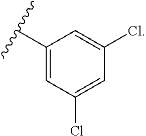

In another embodiment of this invention R¹ is:

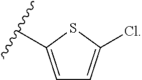

In another embodiment of this invention R¹ is:

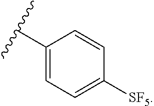

In another embodiment of this invention R¹ is:

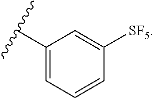

In another embodiment of this invention R¹ is:

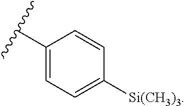

In another embodiment of this invention R¹ is:

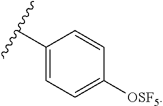

In another embodiment of this invention R¹ is:

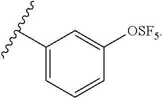

In one preferred embodiment of the invention, R¹ and R⁶ are taken together with the carbon atom to which they are bound to form a spirocyclic carbocyclic moiety or a spirocyclic heterocyclic moiety. Examples of such spirocyclic moieties are selected from the group consisting of:

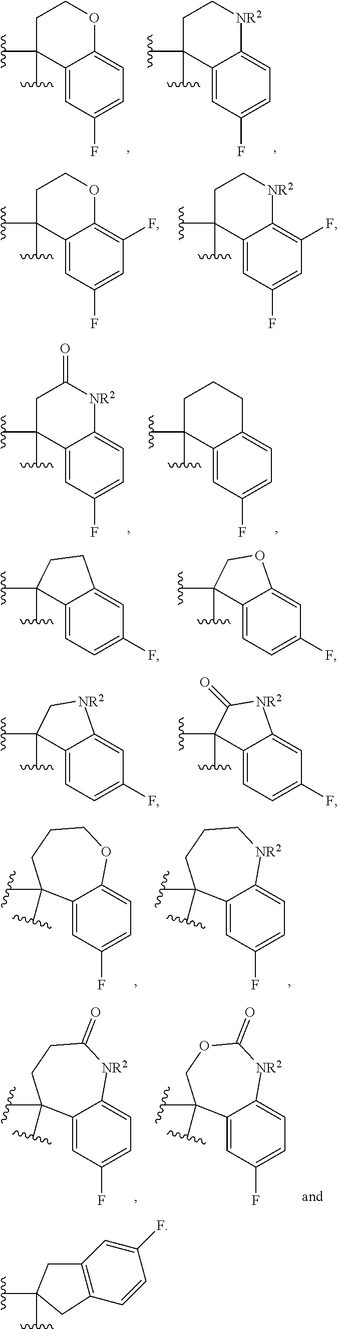

In another embodiment of this invention R¹⁰ is selected from the group consisting of aryl and aryl substituted with one or more R²¹ groups.

In another embodiment of this invention R⁹ is selected from the group consisting of heteroaryl and heteroaryl substituted with one or more R²¹ groups, and wherein each R²¹ is independently selected.

In another embodiment of this invention R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

Other embodiments of this invention are directed to any one of the embodiments above directed to Ring (B) wherein R⁶ is H or alkyl, R¹ is as defined in any one of the above embodiments directed to R¹, R¹⁰ is selected from the group consisting of aryl and aryl substituted with one or more independently selected R²¹ groups, and R⁹ is selected from the group consisting of heteroaryl and heteroaryl substituted with one or more independently selected R²¹ groups. In one such embodiment R⁶ is H or a C₁ to C₃ alkyl group. In another embodiment R⁶ is H or methyl. In another embodiment R⁶ is H or ethyl. In another embodiment R⁶ is H or a C₃ alkyl group. In another embodiment R⁶ is H or isopropyl.

In another embodiment of this invention R¹⁰ is heteroaryl (e.g. pyridyl).

In another embodiment of this invention R¹⁰ is heteroaryl substituted with one or more R²¹ groups (e.g. pyridyl substituted with one or more R²¹ groups).

In another embodiment of this invention R¹⁰ is selected from the group consisting of heteroaryl and heteroaryl substituted with one or more R²¹ groups, and R⁹ group is selected from the group consisting of heteroaryl and heteroaryl substituted with one or more R²¹ groups, and wherein each R²¹ is independently selected.

Other embodiments of this invention are directed to the compounds of Formula I wherein R¹⁰ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Other embodiments of this invention are directed to any one of the embodiments above directed to Ring (B) wherein R⁶ is H or alkyl, R¹ is as defined in any one of the above embodiments directed to R¹, R¹⁰ is selected from the group consisting of heteroaryl and heteroaryl substituted with one or more independently selected R²¹ groups, and R⁹ is selected from the group consisting of heteroaryl and heteroaryl substituted with one or more independently selected R²¹ groups. In one such embodiment R⁶ is H or a C₁ to C₃ alkyl group. In another embodiment R⁶ is H or methyl. In another embodiment R⁶ is H or ethyl. In another embodiment R⁶ is H or a C₃ alkyl group. In another embodiment R⁶ is H or isopropyl.

In another embodiment of this invention R¹⁰ is aryl.

In another embodiment of this invention R¹⁰ aryl is aryl and said aryl is phenyl.

In another embodiment of this invention R¹⁰ is aryl substituted with one or more R²¹ groups.

In another embodiment of this invention R¹⁰ is aryl substituted with one or more R²¹ groups, and said aryl is phenyl, i.e., said R¹⁰ group is phenyl substituted with one or more R²¹ groups.

In another embodiment of this invention R¹⁰ is phenyl substituted with one or more R²¹ groups, and each R²¹ group is the same or different —OR¹⁵ group.

In another embodiment of this invention R¹⁰ is phenyl substituted with one or more R²¹ groups, and each R²¹ group is the same or different —OR¹⁵ group, and said R¹⁵ is alkyl, and each alkyl is independently selected.

In another embodiment of this invention R¹⁰ is phenyl substituted with one R²¹ group, and said R²¹ group is —OR¹⁵, and said R¹⁵ is alkyl.

In another embodiment of this invention R¹⁰ is phenyl substituted with one R²¹ group, and said R²¹ group is —OR¹⁵, and said R¹⁵ is alkyl, and said alkyl is methyl.

In another embodiment of this invention R¹⁰ is phenyl substituted with one or more (e.g., one or two, or one) independently selected R²¹ halo groups.

In another embodiment of this invention R¹⁰ is phenyl substituted with one R²¹ group, and said R²¹ group is halo.

In another embodiment of this invention R¹⁰ is phenyl substituted with one R²¹ group, and said R²¹ group is F.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is halo, and n is 1 to 3, and each halo is independently selected.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is F, and n is 3.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is F, and n is 3, and the alkyl is methyl (i.e., the R²¹ substituent is —OCF₃).

In another embodiment of this invention R⁹ is heteroaryl.

In another embodiment of this invention R⁹ is heteroaryl substituted with one or more R²¹ groups.

In another embodiment of this invention R⁹ is heteroaryl substituted with one or more R²¹ groups, and said R²¹ groups are the same or different alkyl.

In another embodiment of this invention R⁹ is heteroaryl substituted with one R²¹ group, and said R²¹ is alkyl.

In another embodiment of this invention R⁹ is heteroaryl substituted with one R²¹ group, and said R²¹ is alkyl, and said alkyl is methyl.

In another embodiment of this invention R⁹ is and said heteroaryl is imidazoyl.

In another embodiment of this invention R⁹ is imidazolyl substituted with one or more R²¹ groups.

In another embodiment of this invention R⁹ is imidazolyl substituted with one or more R²¹ groups, and said R²¹ groups are the same or different alkyl.

In another embodiment of this invention R⁹ is imidazolyl substituted with one R²¹ group, and said R²¹ is alkyl.

In another embodiment of this invention R⁹ is imidazolyl substituted with one R²¹ group, and said R²¹ is alkyl, and said alkyl is methyl.

In another embodiment of this invention R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

In another embodiment of this invention R¹⁰ is phenyl substituted with one or more R²¹ groups, and said R⁹ is imidazolyl substituted with one or more R²¹ groups, wherein each R²¹ is independently selected.

In another embodiment of this invention R¹⁰ is phenyl substituted with one R²¹ group, and said R⁹ is imidazolyl substituted with one R²¹ group, wherein each R²¹ is independently selected.

In another embodiment of this invention R¹⁰ is phenyl substituted with one or more independently selected —OR¹⁵ groups, and said R⁹ is imidazolyl substituted with one or more independently selected alkyl groups.

In another embodiment of this invention R¹⁰ is phenyl substituted with one or more independently selected —OR¹⁵ groups, and said R⁹ is imidazolyl substituted with one or more independently selected alkyl groups, and each R¹⁵ is the same or different alkyl group.

In another embodiment of this invention R¹⁰ is phenyl substituted with one —OR¹⁵ group, and said R⁹ is imidazolyl substituted with one alkyl group.

In another embodiment of this invention R¹⁰ is phenyl substituted with one —OR¹⁵ group, and said R⁹ is imidazolyl substituted with one alkyl group, and R¹⁵ is alkyl, and wherein the R¹⁵ alkyl group, and the alkyl group on said imidazolyl are independently selected.

In another embodiment of this invention R¹⁰ is phenyl substituted with one —OR¹⁵ group, and said R⁹ is imidazolyl substituted with one methyl group, and R¹⁵ is methyl, and wherein the R¹⁵ alkyl group, and the alkyl group on said imidazolyl are independently selected.

Other embodiments of the compounds of Formula I are directed to any one of the above embodiments wherein R⁹ is:

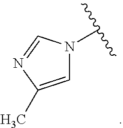

Other embodiments of the compounds of Formula I are directed to any one of the above embodiments wherein R¹⁰ is:

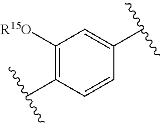

(wherein the —OR¹⁵ is ortho to the carbon to which R⁹ is bound to, i.e., the R⁹-R¹⁰-moiety is:

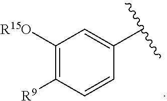

Other embodiments for the compounds of Formula I are directed to any one of the above embodiments wherein R¹⁰ is:

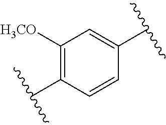

(wherein the —OCH₃ is ortho to the carbon to which R⁹ is bound to, i.e., the R⁹-R¹⁰-moiety is:

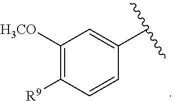

In another embodiment of this invention the R⁹-R¹⁰-moiety is:

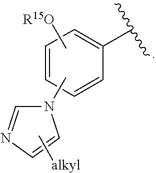

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

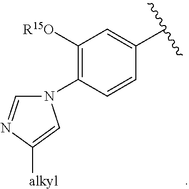

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

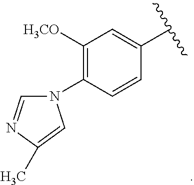

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

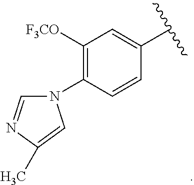

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

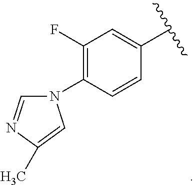

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

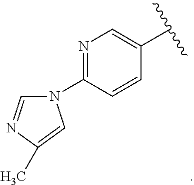

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

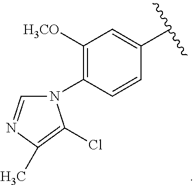

Preferably, in this embodiment G is selected from the group consisting of —NH—, —O—, —S—, —S(O)—, —S(O)₂— and a direct bond, and most preferably, a direct bond.

In another embodiment of this invention the R⁹-R¹⁰-moiety is:

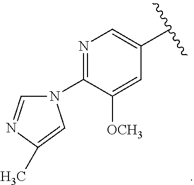

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

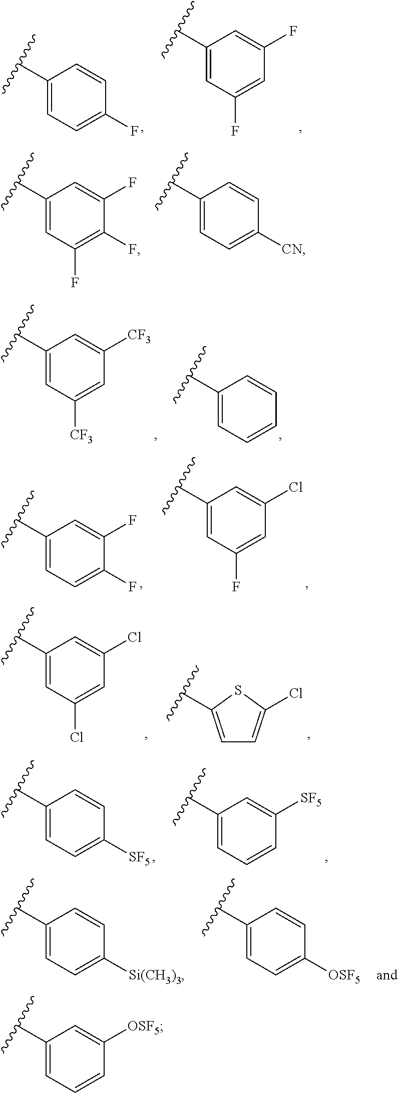

and the R⁹-R¹⁰-moiety is selected from the group consisting of:

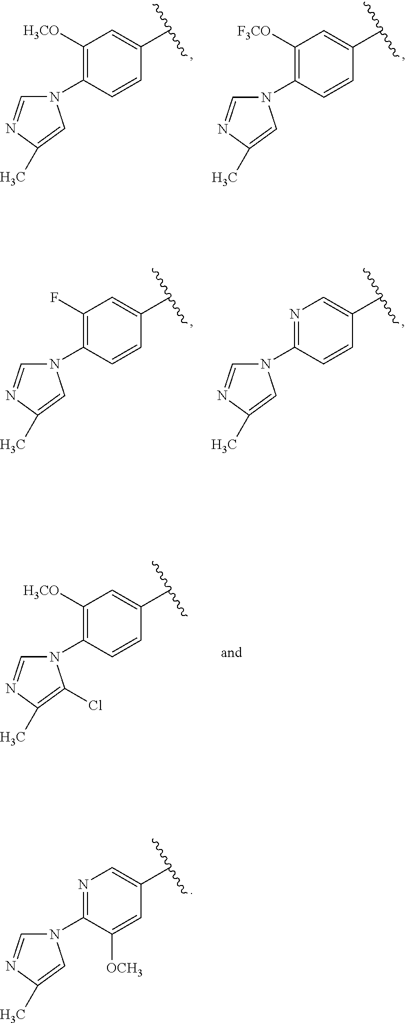

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

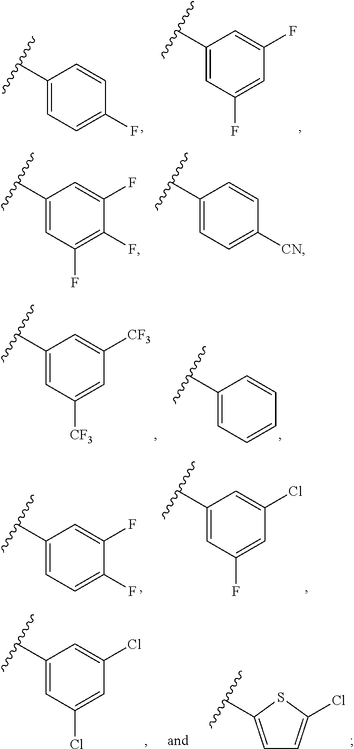

and
the R⁹-R¹⁰-moiety is selected from the group consisting of:

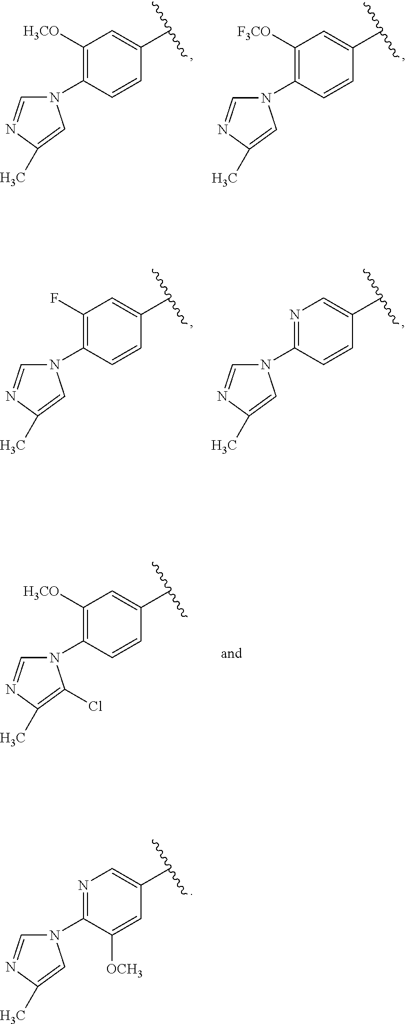

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

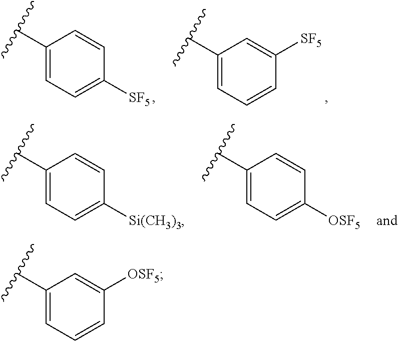

and
the R⁹-R¹⁰-moiety is selected from the group consisting of:

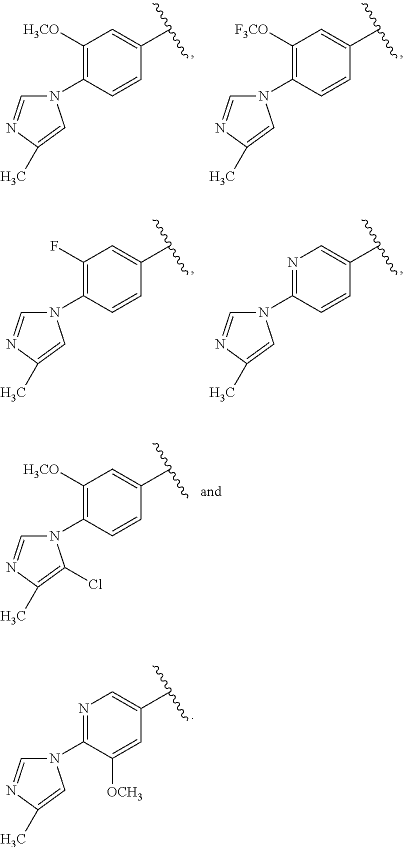

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

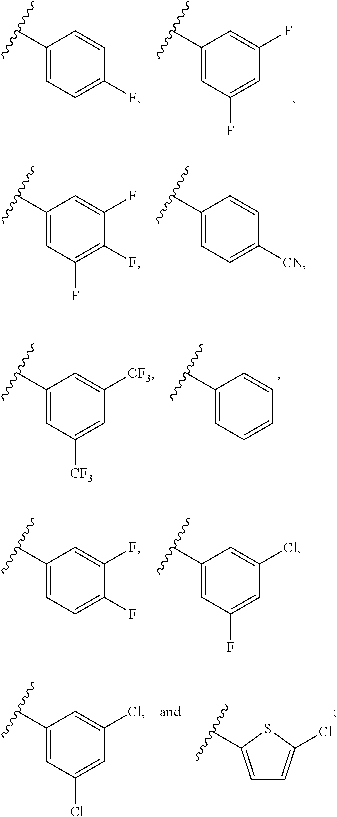

and
the R⁹-R¹⁰-moiety is selected from the group consisting of:

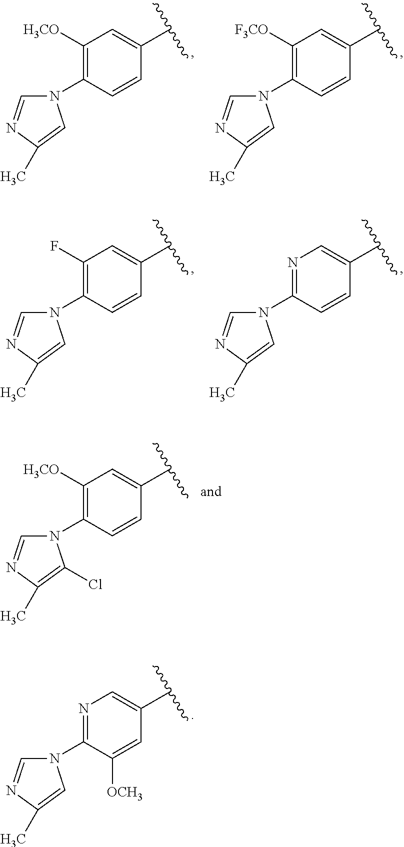

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

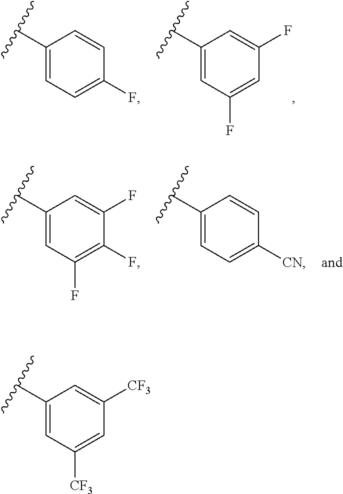

the R⁹-R¹⁰-moiety is selected from the group consisting of:

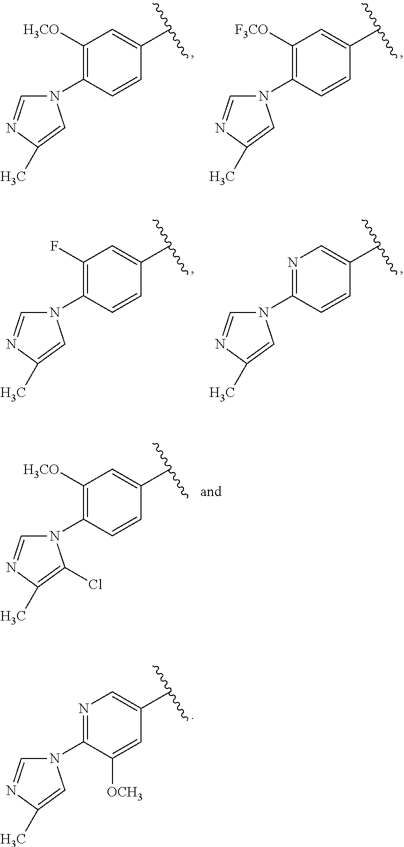

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

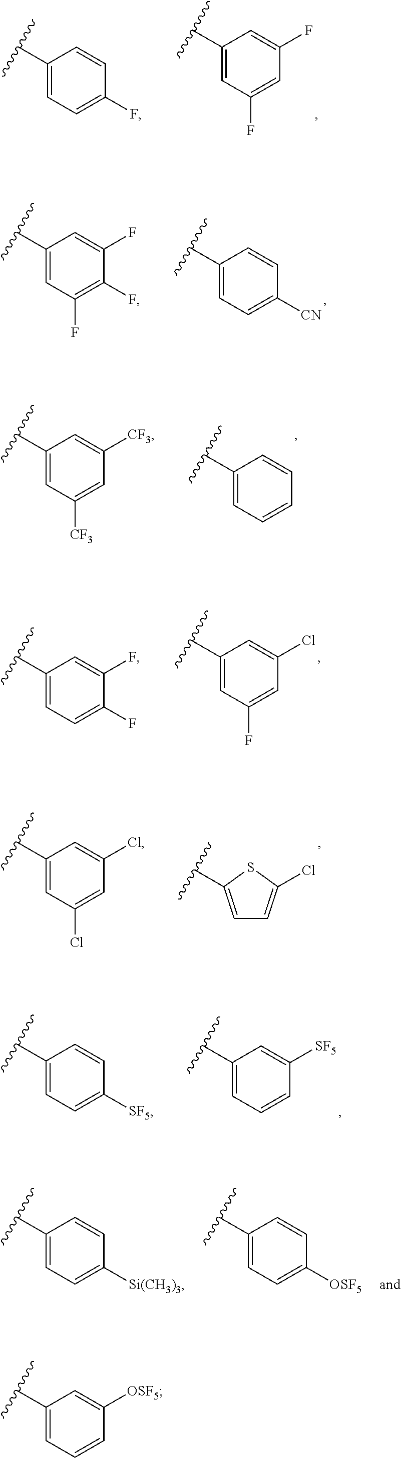

and
the R⁹-R¹⁰-moiety is:

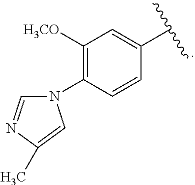

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

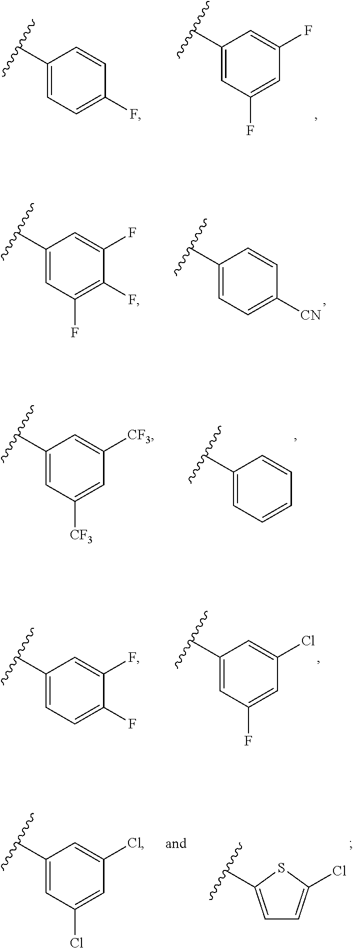

and
the R⁹-R¹⁰-moiety is:

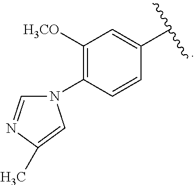

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

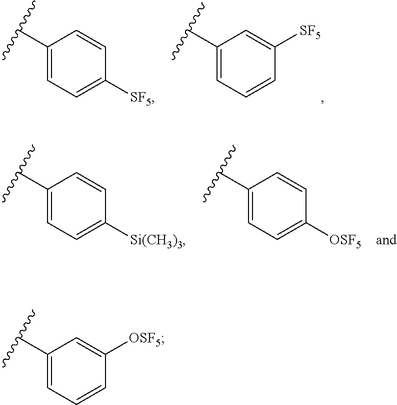

and
the R⁹-R¹⁰-moiety is:

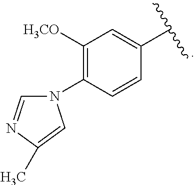

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

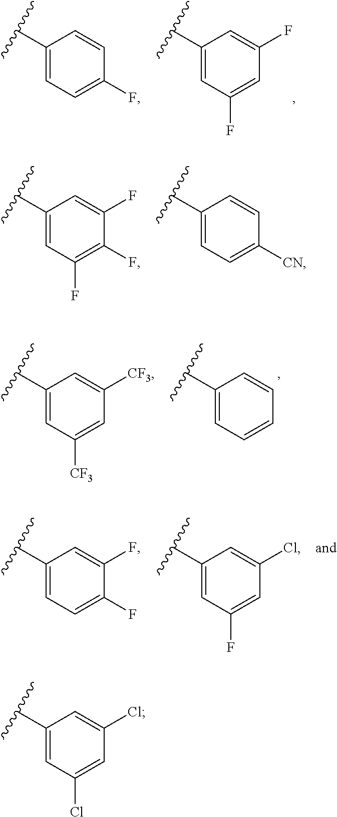

and
the R⁹-R¹⁰-moiety is:

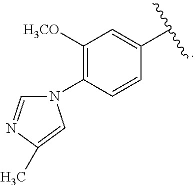

Preferably, in this embodiment G is selected from the group consisting of —NH—, —O—, —S—, —S(O)—, —S(O)₂— and a direct bond, and most preferably, a direct bond.

In another embodiment of this invention R⁶ is H or alkyl; R¹ is selected from the group consisting of:

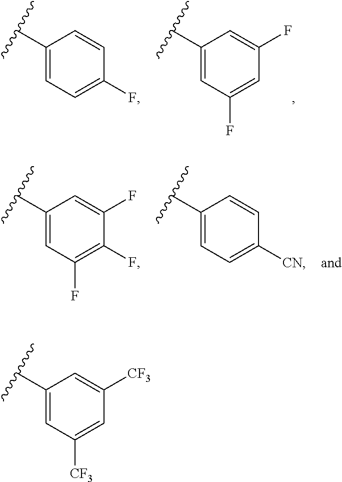

the R⁹-R¹⁰-G moiety is:

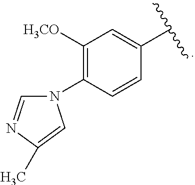

Preferably, in this embodiment G is selected from the group consisting of —NH—, —O—, —S—, —S(O)—, —S(O)₂— and a direct bond, and most preferably, a direct bond.

In another embodiment of this invention: (a) R⁶ is H or alkyl (e.g., methyl), (b) R¹ is an aryl group, or R¹ is an aryl group substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R⁶ is H or alkyl (e.g., methyl), (b) R¹ is phenyl, or R¹ is phenyl substituted with 1 to 3 independently selected R²¹ halo groups, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

In another embodiment of this invention: (a) R⁶ is H or alkyl (e.g., methyl), (b) R¹ is phenyl, or R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is H or alkyl (e.g., methyl), (b) R¹ is phenyl, or R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is H or alkyl (e.g., methyl), (b) R¹ is phenyl, or R¹ is phenyl, substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, or R¹ is phenyl substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, or R¹ is phenyl substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R⁶ is H or alkyl (e.g., methyl), (b) R¹ is phenyl substituted with 1 to 3 independently selected R²¹ halo groups, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

In another embodiment of this invention: (a) R⁶ is H or alkyl (e.g., methyl), (b) R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is H or alkyl (e.g., methyl), (b) R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is H or alkyl (e.g., methyl), (b) R¹ is phenyl, substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is H or alkyl (e.g., methyl), (b) R¹ is phenyl, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R⁶ is H or alkyl (e.g., methyl), (b) R¹ is phenyl, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is phenyl, (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is H or alkyl (e.g., methyl), (b) R¹ is an aryl group, or R¹ is an aryl group substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is O.

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is O.

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is O.

In another embodiment of this invention: (a) R⁶ is H or alkyl (e.g., methyl), (b) R¹ is phenyl, or R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is O.

In another embodiment of this invention: (a) R⁶ is H or alkyl (e.g., methyl), (b) R¹ is phenyl, or R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, (e) and G is O.

In another embodiment of this invention: (a) R⁶ is H or alkyl (e.g., methyl), (b) R¹ is phenyl, or R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is O.

In another embodiment of this invention: (a) R⁶ is H or alkyl (e.g., methyl), (b) R¹ is phenyl, or R¹ is phenyl, substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl, or R¹ is phenyl, substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl, or R¹ is phenyl, substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is O.

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is O.

In another embodiment of this invention: (a) R⁶ is H or alkyl (e.g., methyl), (b) R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is O.

In another embodiment of this invention: (a) R⁶ is H or alkyl (e.g., methyl), (b) R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is O.

In another embodiment of this invention: (a) R⁶ is H or alkyl (e.g., methyl), (b) R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is O.

In another embodiment of this invention: (a) R⁶ is H or alkyl (e.g., methyl), (b) R¹ is phenyl substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl, substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is O.

In another embodiment of this invention: (a) R⁶ is H or alkyl (e.g., methyl), (b) R¹ is phenyl, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is O.

In another embodiment of this invention: (a) R⁶ is H or alkyl (e.g., methyl), (b) R¹ is phenyl, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is O.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is O.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is O.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is phenyl, (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is O.

In another embodiment of this invention: (a) R⁶ is H or alkyl (e.g., methyl), (b) R¹ is an aryl group, or R¹ is an aryl group substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is S.

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is S.

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is S.

In another embodiment of this invention: (a) R⁶ is H or alkyl (e.g., methyl), (b) R¹ is phenyl, or R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is S.

In another embodiment of this invention: (a) R⁶ is H or alkyl (e.g., methyl), (b) R¹ is phenyl, or R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, (e) and G is S.

In another embodiment of this invention: (a) R⁶ is H or alkyl (e.g., methyl), (b) R¹ is phenyl, or R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is S.

In another embodiment of this invention: (a) R⁶ is H or alkyl (e.g., methyl), (b) R¹ is phenyl, or R¹ is phenyl, substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl, or R¹ is phenyl, substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl, or R¹ is phenyl substituted with 1 F (i.e., R¹ is phenyl substituted with 1 R¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is S.

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is S.

In another embodiment of this invention: (a) R⁶ is H or alkyl (e.g., methyl), (b) R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is S.

In another embodiment of this invention: (a) R⁶ is H or alkyl (e.g., methyl), (b) R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is S.

In another embodiment of this invention: (a) R⁶ is H or alkyl (e.g., methyl), (b) R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is S.

In another embodiment of this invention: (a) R⁶ is H or alkyl (e.g., methyl), (b) R¹ is phenyl substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl, substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is S.

In another embodiment of this invention: (a) R⁶ is H or alkyl (e.g., methyl), (b) R¹ is phenyl, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is S.

In another embodiment of this invention: (a) R⁶ is H or alkyl (e.g., methyl), (b) R¹ is phenyl, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is S.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is S.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is S.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is phenyl, (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is S.

In another embodiment of this invention: (a) R⁶ is H or alkyl (e.g., methyl), (b) R¹ is an aryl group, or R¹ is an aryl group substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is C(O).

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is C(O).

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is C(O).

In another embodiment of this invention: (a) R⁶ is H or alkyl (e.g., methyl), (b) R¹ is phenyl, or R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is C(O).

In another embodiment of this invention: (a) R⁶ is H or alkyl (e.g., methyl), (b) R¹ is phenyl, or R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, (e) and G is C(O).

In another embodiment of this invention: (a) R⁶ is H or alkyl (e.g., methyl), (b) R¹ is phenyl, or R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is C(O).

In another embodiment of this invention: (a) R⁶ is H or alkyl (e.g., methyl), (b) R¹ is phenyl, or R¹ is phenyl substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl, or R¹ is phenyl substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl, or R¹ is phenyl substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is C(O).

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is C(O).

In another embodiment of this invention: (a) R⁶ is H or alkyl (e.g., methyl), (b) R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is C(O).

In another embodiment of this invention: (a) R⁶ is H or alkyl (e.g., methyl), (b) R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is C(O).

In another embodiment of this invention: (a) R⁶ is H or alkyl (e.g., methyl), (b) R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is C(O).

In another embodiment of this invention: (a) R⁶ is H or alkyl (e.g., methyl), (b) R¹ is phenyl, substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is C(O).

In another embodiment of this invention: (a) R⁶ is H or alkyl (e.g., methyl), (b) R¹ is phenyl, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is C(O).

In another embodiment of this invention: (a) R⁶ is H or alkyl (e.g., methyl), (b) R¹ is phenyl, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is C(O).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is C(O).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is C(O).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is phenyl, (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is C(O).

In another embodiment of this invention: (a) R⁶ is H or alkyl (e.g., methyl), (b) R¹ is an aryl group, or R¹ is an aryl group substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, (e) and G is N(R¹⁴) (and in one example G is NH).

In another embodiment of this invention: (a) R⁶ is H or alkyl (e.g., methyl), (b) R¹ is phenyl, or R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, or R¹ is phenyl substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl substituted with 1 to 3 independently selected R²¹ groups, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl substituted with 1 to 3 independently selected R²¹ halo groups, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is N(R¹⁴) (and in one example G is NH).

In another embodiment of this invention: (a) R⁶ is H or alkyl (e.g., methyl), (b) R¹ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is N(R¹⁴) (and in one example G is NH).

In another embodiment of this invention: (a) R⁶ is H or alkyl (e.g., methyl), (b) R¹ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is N(R¹⁴) (and in one example G is NH).

In another embodiment of this invention: (a) R⁶ is H or alkyl (e.g., methyl), (b) R¹ is phenyl, substituted with 1 to 3 F (i.e., R¹ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, substituted with 1 to 2 F (i.e., R¹ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, substituted with 1 F (i.e., R¹ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of aryl and aryl substituted with one or more independently selected R²¹ groups, (d) R⁹ is selected from the group consisting of heteroaryl and heteroaryl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of phenyl and phenyl substituted with one or more independently selected R²¹ groups, (d) R⁹ is selected from the group consisting of imidazolyl and imidazolyl substituted with one or more independently selected R²¹ groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of phenyl and phenyl substituted with one or more independently selected —OR¹⁵ groups, (d) R⁹ is selected from the group consisting of imidazolyl and imidazolyl substituted with one or more independently selected alkyl groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is alkyl, (d) R⁹ is selected from the group consisting of imidazolyl and imidazolyl substituted with one or two independently selected alkyl groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, (c) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

In another embodiment of this invention: (a) R⁶ is H or alkyl (e.g., methyl), (b) R¹ is phenyl, (b) R¹⁰ is selected from the group consisting of phenyl and phenyl substituted with one or two independently selected —OR¹⁵ groups, wherein R¹⁵ is methyl, (d) R⁹ is selected from the group consisting of imidazolyl and imidazolyl substituted with one or two independently selected methyl groups, and (e) G is N(R¹⁴) (and in one example G is NH).

Other embodiments of this invention are directed to the above embodiments (which are directed to the combination of R⁶, and R¹, and R¹⁰, and R⁹, with G being O, C(O), N(R¹⁴) (such as NH), or S) wherein ring B is as defined in any one of the embodiments directed to ring B above.

Other embodiments of this invention are directed to the above embodiments (which are directed to the combination of R⁶, and R¹, and R¹⁰, and R⁹, with G being O, C(O), N(R¹⁴) (such as NH), or S) wherein the optional bond in ring B is present.

Other embodiments of this invention are directed to the above embodiments (which are directed to the combination of R⁶, and R¹, and R¹⁰, and R⁹, with G being O, C(O), N(R¹⁴) (such as NH), or S) wherein the optional bond in ring B is present and U is N.

Other embodiments of this invention are directed to the above embodiments (which are directed to the combination of R⁶, and R¹, and R¹⁰, and R⁹, with G being O, C(O), N(R¹⁴) (such as NH), or S) wherein the optional bond in ring B is present and U is CR⁵.

Other embodiments of this invention are directed to the above embodiments (which are directed to the combination of R⁶, and R¹, and R¹⁰, and R⁹, with G being O, C(O), N(R¹⁴) (such as NH), or S) wherein the optional bond in ring B is present, U is CR⁵, and ring B is a 7 membered ring.

Other embodiments of this invention are directed to the above embodiments (which are directed to the combination of R⁶, and R¹, and R¹⁰, and R⁹, with G being O, C(O), N(R¹⁴) (such as NH), or S) wherein the optional bond in ring B is present, U is CR⁵ and R⁵ is H.

Other embodiments of this invention are directed to the above embodiments (which are directed to the combination of R⁶, and R¹, and R¹⁰, and R⁹, with G being O, C(O), N(R¹⁴) (such as NH), or S) wherein the optional bond in ring B is present, U is CR⁵ and R⁵ is H, and ring B is a 7 membered ring.

Other embodiments of this invention are directed to any one of the embodiments above wherein ring B is substituted with 1 or two R²¹ groups. In one example each R²¹ group is the same or different alkyl group. In another example each R²¹ is methyl. In another example ring B is substituted with two R²¹ groups. In another example ring B is substituted with two R²¹ groups and each group is the same or different alkyl group. In another example ring B is substituted with two R²¹ groups and each group is methyl group. In another example ring B is substituted with one R²¹ group. In another example ring B is substituted with one R²¹ group and said R²¹ group is alkyl. In another example ring B is substituted with one R²¹ group and said R²¹ group is alkyl and said alkyl group is methyl.

In another embodiment of this invention ring B is substituted with 1 or two R²¹ groups.

In another embodiment of this invention ring B is substituted with 1 or 2 R²¹ groups wherein each R²¹ group is the same or different alkyl group.

In another embodiment of this invention ring B is substituted with 1 or 2 R²¹ groups wherein each R²¹ group is methyl.

In another embodiment of this invention ring B is substituted with two R²¹ groups.

In another embodiment of this invention ring B is substituted with two R²¹ groups wherein each R²¹ group is the same or different alkyl group.

In another embodiment of this invention ring B is substituted with two R²¹ groups and each R²¹ group is methyl.

In another embodiment of this invention ring B is substituted with

In another embodiment of this invention R²¹ is selected from the group consisting of: alkyl, —OR⁵, —C(O)OR¹⁵, —C(O)NR¹⁵R¹⁶, and alkyl substituted with 1 to 5 independently selected R²² groups (e.g., halo, such as, for example, F, Cl, and Br).

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br, and wherein in one example the alkyl substituted R²¹ group is —CF₃), wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g, n is 1, R¹⁸ is —OR²⁰, and R²⁰ is alkyl (e.g., methyl).

In another embodiment of this invention R²¹ is selected from the group consisting of: (a) alkyl, —OR¹⁵ (wherein R¹⁵ is alkyl, e.g., methyl and ethyl), (b) —C(O)OR⁵ (wherein R¹⁵ is alkyl, e.g., methyl), (c) —C(O)NR¹⁵R¹⁶ (wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g, n is 1, R¹⁸ is —OR²⁰, and R²⁰ is alkyl (e.g., methyl), and in one example, only one of R¹⁵ and R¹⁶ is H), and (d) alkyl substituted with 1 to 5 independently selected R²² groups (e.g., halo, such as, for example, F, Cl, and Br, and wherein in one example the alkyl substituted R²¹ group is —CF₃).

Other embodiments of this invention are directed to compounds of Formula I wherein R⁶ or R¹ is a cycloalkyl group (e.g., cyclopropyl or cyclobutyl) substituted with one R²¹ group (e.g., aryl, such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F). In one example the R²¹ group is bound to the same carbon of the R⁶ or R¹ group that binds the R⁶ or R¹ group to the rest of the molecule.

Examples of the cycloalkyl R⁶ or R¹ groups include, but are not limited to:

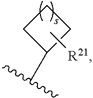

such as, for example,

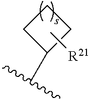

wherein s is 0 (i.e., the ring is cyclopropyl), or 1 (i.e., the ring is cyclobutyl). Examples of these R⁶ or R¹ groups include, but are not limited to:

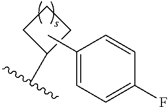

such as, for example,

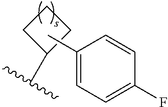

wherein s is 0 (i.e., the ring is cyclopropyl), or 1 (i.e., the ring is cyclobutyl).

Other embodiments of this invention are directed to compounds of Formula I wherein R⁶ or R¹ is

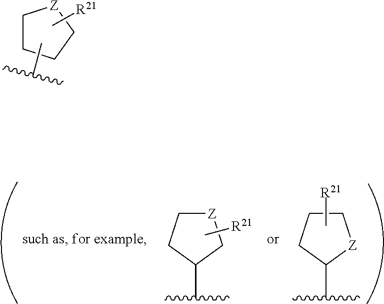

wherein Z is selected from the group consisting of: (1) —O—, (2) —NR¹⁴—, (3) —C(R²¹)_{q}— wherein q is 0, 1 or 2, and each R²¹ is independently selected, (4) —C(R²¹)_{q}—C(R²¹)_{q}— wherein each q is independently 0, 1 or 2 and each R²¹ is independently selected, (5) —(C(R²¹)_{q})_{q}—O—(C(R²¹)_{q})_{q}— wherein each q is independently 0, 1 or 2, and each R²¹ is independently selected, and (6) —(C(R²¹)_{q})_{q}—N(R¹⁴)—(C(R²¹)_{q})_{q}— wherein each q is independently 0, 1 or 2, and each R²¹ is independently selected. Examples of R²¹ include, but are not limited to, aryl (e.g., phenyl) and aryl (e.g., phenyl) substituted with one or more (e.g., one or two, or one) independently selected R²² groups (e.g., halo, such as, for example, F). Examples of this R⁶ or R¹ include, but are not limited to:

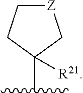

Thus, examples of this R⁶ or R¹ group include, but are not limited to:

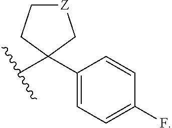

Examples of R⁶ or R¹ also include, but are not limited to:

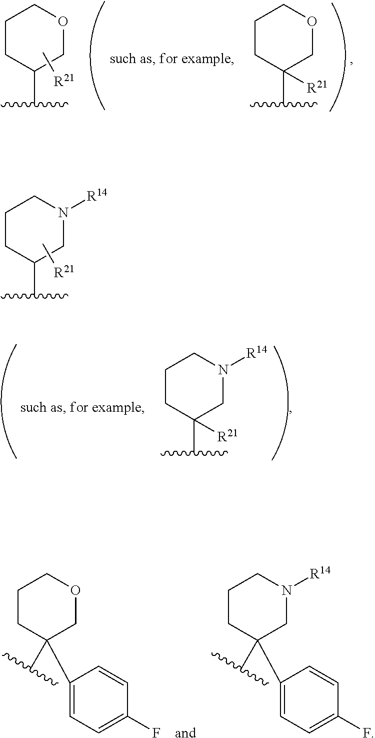

Examples of the R⁶ or R¹ group

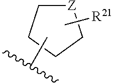

also include, but are not limited to:

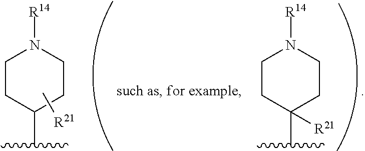

Examples of the R⁶ or R¹ group

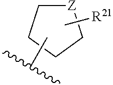

also include, but are not limited to:

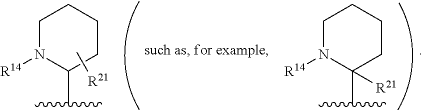

Examples of the R⁶ or R¹ group

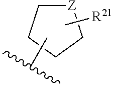

also include, but are not limited to:

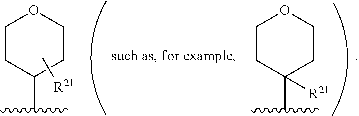

Examples of the R⁶ or R¹ group

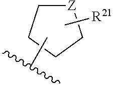

also include, but are not limited to:

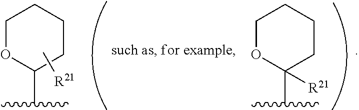

Other embodiments of this invention are directed to compounds of Formula I wherein R¹⁰ is aryl (e.g., phenyl) or aryl (e.g., phenyl) substituted with one or more (e.g., one or two, or one) R²¹ groups (e.g., —OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, examples of the —R¹⁰-R⁹ moiety of the compounds of this invention include, but are not limited to:

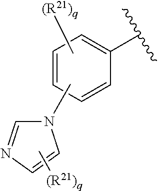

wherein q is 0, 1 or 2, such as, for example,

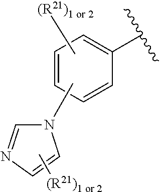

such as, for example,

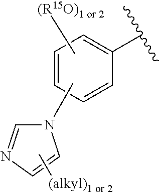

wherein R¹⁵ is alkyl (e.g., methyl), such as, for example,

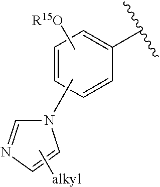

wherein R¹⁵ is alkyl (e.g., methyl), such as, for example,

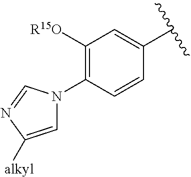

wherein R¹⁵ is alkyl (e.g., methyl), such as, for example,

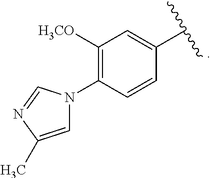

In the above examples of R⁹-R¹⁰—, G is preferably selected from the group consisting of: —C(O)—, —CH₂—, CF₂, —CH═CH—, —C═C—, —C(O)NR⁵—, —NR⁵C(O)—, —(C═NR²)—, —S(O)ₜNR⁵—, —NR⁵S(O)ₜ—, —NH—, —O—, —S—, —S(O)—, —S(O)₂— and a direct bond, more preferably, G is selected from the group consisting of —NH—, —O—, —S—, —S(O)—, —S(O)₂— and a direct bond, and most preferably G is a direct bond.

In another embodiment of the compounds of Formula I R⁶ or R¹ is benzofusedcycloalkyl.

In another embodiment of the compounds of Formula I R⁶ or R¹ is:

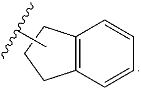

In another embodiment of the compounds of Formula I R⁶ or R¹ is:

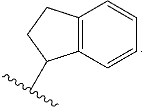

In another embodiment of the compounds of Formula I R⁶ or R¹ is:

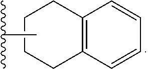

In another embodiment of the compounds of Formula I R⁶ or R¹ is:

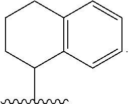

In another embodiment of the compounds of Formula I R⁶ or R¹ is alkyl substituted with one R²¹ group.

In another embodiment of the compounds of Formula I R⁶ or R¹ is alkyl substituted with one R²¹ group, and said alkyl is

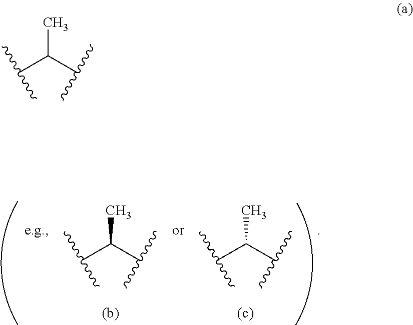

In another embodiment of the compounds of Formula I R⁶ or R¹ is alkyl (e.g., (a), (b) or (c) described above) substituted with one R²¹ group wherein said R²¹ group is aryl.

In another embodiment of the compounds of Formula I R⁶ or R¹ is alkyl (e.g., (a), (b) or (c) described above) substituted with one R²¹ group wherein said R²¹ group is phenyl.

In another embodiment of the compounds of Formula I R⁶ or R¹ is alkyl (e.g., (a), (b) or (c) described above) substituted with one R²¹ group wherein said R²¹ group is naphthyl.

In another embodiment of the compounds of Formula I R⁶ or R¹ is alkyl substituted with one R²¹ group, and said R²¹ group is substituted with two independently selected R²² groups.

In another embodiment of the compounds of Formula I R⁶ or R¹ is alkyl substituted with one R²¹ group, and said R²¹ group is substituted with one R²² group.

In another embodiment of the compounds of Formula I R⁶ or R¹ is alkyl substituted with one R²¹ group, wherein said alkyl group is (a) (e.g., (b) or (c)), as described above, and said R²¹ group is substituted with two independently selected R²² groups.

In another embodiment of the compounds of Formula I R⁶ or R¹ is alkyl substituted with one R²¹ group, wherein said alkyl group is (a) (e.g., (b) or (c)), as described above, and said R²¹ group is substituted with one R²² group.

In another embodiment of the compounds of Formula I R⁶ or R¹ is alkyl substituted with one R²¹ group, wherein said R²¹ group is aryl, and said R²¹ group is substituted with two independently selected R²² groups.

In another embodiment of the compounds of Formula I R⁶ or R¹ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of Formula I R⁶ or R¹ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of Formula I R⁶ or R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of Formula I R⁶ or R¹ is alkyl substituted with one R²¹ group, wherein said R²¹ group is aryl, wherein said alkyl group is (a) (e.g., (b) or (c)), as described above, and said R²¹ group is substituted with one R²² group and said R²² is halo.

In another embodiment of the compounds of Formula I R⁶ or R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of Formula I R⁶ or R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of the compounds of Formula I R⁶ or R¹ is:

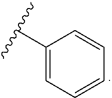

In another embodiment of the compounds of Formula I R⁶ or R¹ is:

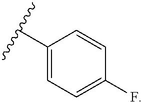

In another embodiment of the compounds of Formula I R⁶ or R¹ is:

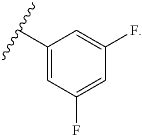

In another embodiment of this invention R⁶ or R¹ is:

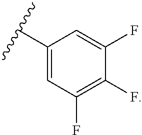

In another embodiment of this invention R⁶ or R¹ is:

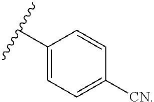

In another embodiment of this invention R⁶ or R¹ is:

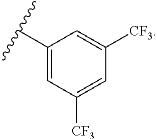

In another embodiment of this invention R⁶ or R¹ is:

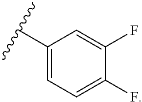

In another embodiment of this invention R⁶ or R¹ is:

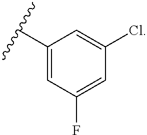

In another embodiment of this invention R⁶ or R¹ is:

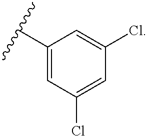

In another embodiment of this invention R⁶ or R¹ is:

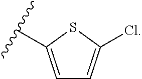

In another embodiment of this invention R⁶ or R¹ is:

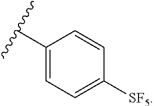

In another embodiment of this invention R⁶ or R¹ is:

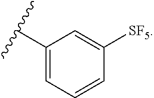

In another embodiment of this invention R⁶ or R¹ is:

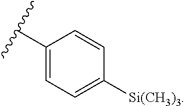

In another embodiment of this invention R⁶ or R¹ is:

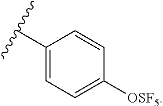

In another embodiment of this invention R⁶ or R¹ is:

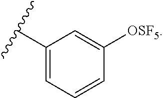

Examples of R²¹ groups include —OR¹⁵ wherein, for example, R¹⁵ is alkyl (such as methyl or ethyl), or R¹⁵ is cycloalkylalkyl (such as, for example, —CH₂-cyclopropyl), or R¹⁵ is -alkyl-(R¹⁸)ₙ (wherein, for example, said R¹⁸ is —OR²⁰, and said R²⁰ is alkyl, and wherein examples of said -alkyl-(R¹⁸)ₙ moiety is —(CH₂)₂OCH₃).

Examples of the R²¹ moiety in the embodiments of this invention include, but are not limited to: (a) —OR¹⁵, (b) —OR¹⁵ wherein R¹⁵ is alkyl, (c) —OR¹⁵ wherein R¹⁵ is alkyl and said alkyl is methyl or ethyl, (d) —OR¹⁵ wherein R¹⁵ is cycloalkylalkyl, (e) —OR¹⁵ wherein R¹⁵ is -alkyl-(R¹⁸)ₙ, (f) —OR¹⁵ wherein R¹⁵ is -alkyl-(R⁸)ₙ and wherein said R¹⁸ is —OR²⁰, (g) —OR¹⁵ wherein R¹⁵ is -alkyl-(R¹⁸)ₙ and wherein said R¹⁸ is —OR²⁰ and said R²⁰ is alkyl. Examples of the R²¹ moiety include but are not limited to: —OCH₃, —OCH₂CH₃, —O(CH₂)₂OCH₃, and —CH₂-cyclopropyl.

Examples of R²¹ also include —C(O)OR¹⁵ wherein, for example, R¹⁵ is alkyl, such as, for example, methyl).

Examples of R²¹ also include —C(O)NR¹⁵R¹⁶, wherein, for example, one of R¹⁵ or R¹⁶ is H, and the other is selected from the group consisting of: (R¹⁸)ₙ-arylalkyl-, (R¹⁸)ₙ-alkyl-, and cycloalkyl. In one example of this —C(O)NR¹⁵R¹⁶ moiety the R¹⁸ is —OR²⁰, n is 1, R²⁰ is alkyl, said cycloalkyl is cyclobutyl, and said arylalkyl- is benzyl.

Examples of R²¹ also include halo (e.g., Br, Cl or F).

Examples of R²¹ also include arylalkyl, such as, for example, benzyl.

One embodiment of the invention is a compound of represented by the formula:

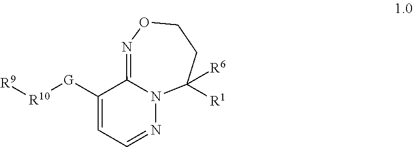

wherein R¹, R⁶, R⁹, R¹⁰ and G are as defined in connection with Formula I above. One example is a compound of the formula:

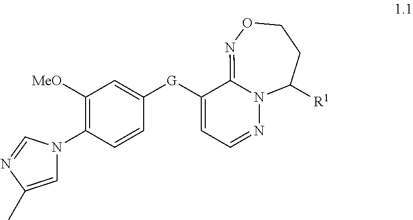

wherein R¹ and G are as defined in connection with Formula I above.

Another embodiment of the invention is a compound of represented by the formula:

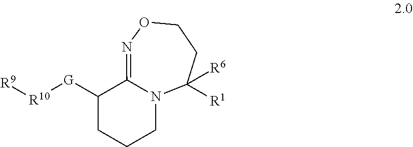

wherein R¹, R⁶, R⁹, R¹⁰ and G are as defined in connection with Formula I above. One example is a compound of the formula:

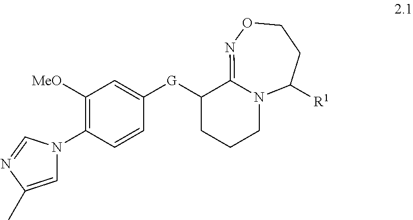

wherein R¹ and G are as defined in connection with Formula I above.

Another embodiment of the invention is a compound of represented by the formula:

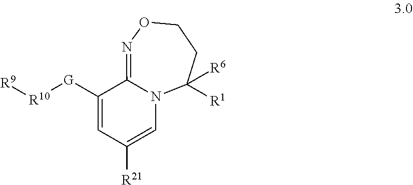

wherein R¹, R⁶, R⁹, R¹⁰, R²¹ and G are as defined in connection with Formula I above. One example is a compound of the formula:

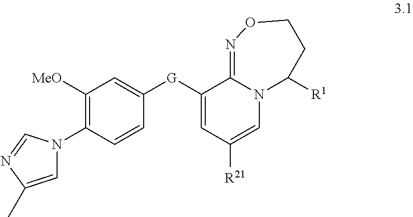

wherein R¹, R²¹ and G are as defined in connection with Formula I above.

Another embodiment of the invention is a compound of represented by the formula:

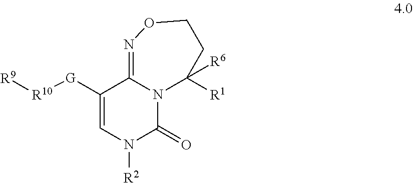

wherein R¹, R², R⁶, R⁹, R¹⁰ and G are as defined in connection with Formula I above. One example is a compound of the formula:

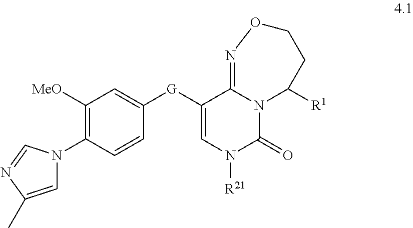

wherein R¹, R² and G are as defined in connection with Formula I above.

Another embodiment of the invention is a compound of represented by the formula:

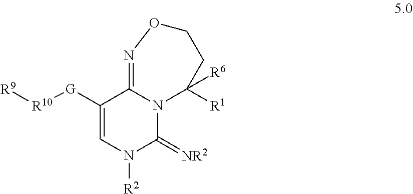

wherein R¹, R² (each R² is independently selected), R⁶, R⁹, R¹⁰ and G are as defined in connection with Formula I above. One example is a compound of the formula:

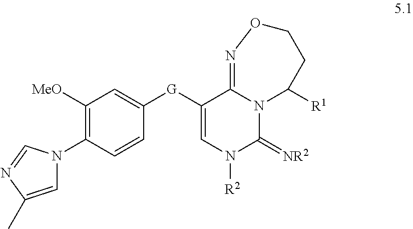

wherein R¹, R² (each R² is independently selected) and G are as defined in connection with Formula I above.

Another embodiment of the invention is a compound of represented by the formula:

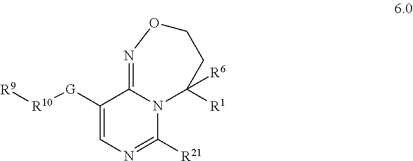

wherein R¹, R⁶, R⁹, R¹⁰, R²¹ and G are as defined in connection with Formula I above.

One example is a compound of the formula:

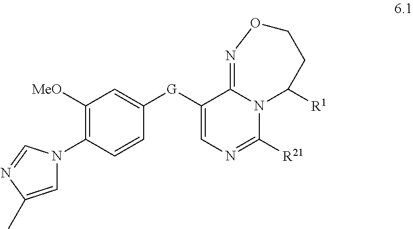

wherein R¹, R²¹ and G are as defined in connection with Formula I above.

Another embodiment of the invention is a compound of represented by the formula:

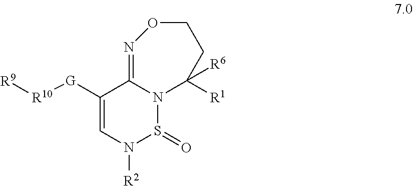

wherein R¹, R⁶, R⁹, R¹⁰, R² and G are as defined in connection with Formula I above.

One example is a compound of the formula:

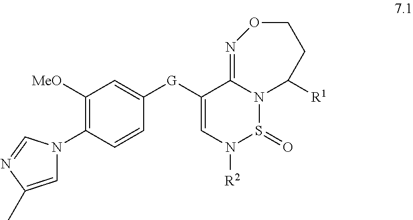

wherein R¹, R² and G are as defined in connection with Formula I above.

Another embodiment of the invention is a compound of represented by the formula:

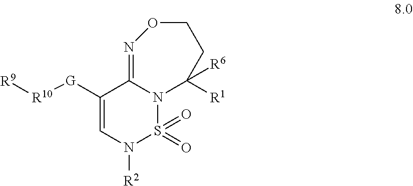

wherein R¹, R⁶, R⁹, R¹⁰, R² and G are as defined in connection with Formula I above. One example is a compound of the formula:

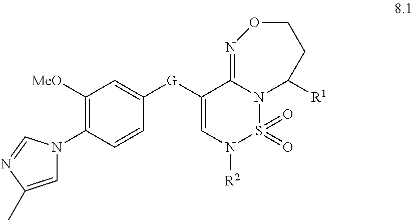

wherein R¹, R² and G are as defined in connection with Formula I above.

Another embodiment of the invention is a compound of represented by the formula:

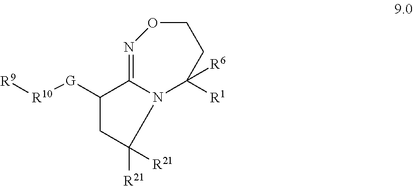

wherein R¹, R⁶, R⁹, R¹⁰, R²¹ (each R²¹ is independently selected) and G are as defined in connection with Formula I above. One example is a compound of the formula:

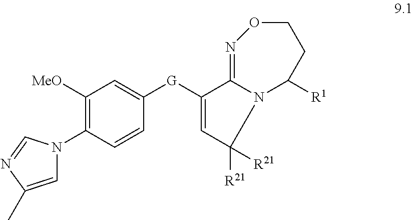

wherein R¹, R²¹ (each R²¹ is independently selected) and G are as defined in connection with Formula I above.

Another embodiment of the invention is a compound of represented by the formula:

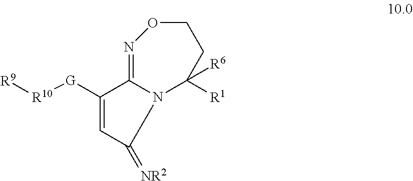

wherein R¹, R², R⁶, R⁹, R¹⁰ and G are as defined in connection with Formula I above. One example is a compound of the formula:

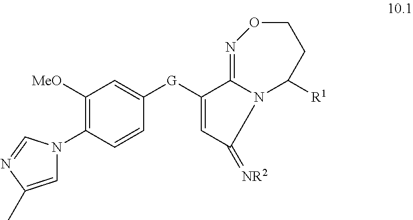

wherein R¹, R² and G are as defined in connection with Formula I above.

Another embodiment of the invention is a compound of represented by the formula:

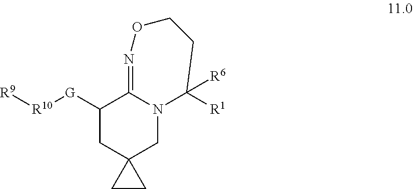

wherein R¹, R⁶, R⁹, R¹⁰ and G are as defined in connection with Formula I above. One example is a compound of the formula:

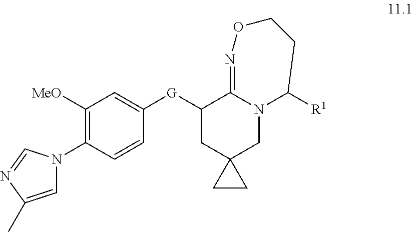

wherein R¹ and G are as defined in connection with Formula I above.

In each embodiment of formulas 1 through 4, 1.0 through 11.0, 1.1 through 11.1 above when the terms R¹, R², R⁶, R⁹, R¹⁰, R²¹ and G are employed:

R¹ is preferably selected from the group consisting of alkyl, alkenyl, alkynyl, cycloalkyl, cycloalkylalkyl-, heterocyclyl (e.g., heterocycloalkyl), cycloalkenyl, arylalkyl-, alkylaryl-, aryl (e.g., phenyl), heteroaryl (e.g., pyridyl), heterocyclenyl (i.e., heterocycloalkenyl), fused cycloalkylaryl (i.e., cycloalkylusedlaryl-), fused heterocycloalkylaryl- (i.e., heterocycloalkylfusedaryl-), fused cycloalkylheteroaryl- (i.e., cycloalkylfusedheteroaryl-) and fused heterocycloalkylheteroaryl- (i.e., heterocycloalkylfusedheteroaryl-), and more preferably selected from the group consisting of alkyl, cycloalkyl, heterocyclyl (e.g., heterocycloalkyl), cycloalkenyl, arylalkyl-, alkylaryl-, aryl (e.g., phenyl), heteroaryl (e.g., pyridyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-) and fused heterocycloalkylheteroaryl- (i.e., heterocycloalkylfusedheteroaryl-), and most preferably selected from the group consisting of alkyl, cycloalkyl, heterocyclyl (e.g., heterocycloalkyl), aryl (e.g., phenyl), heteroaryl (e.g., pyridyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-) and fused heterocycloalkylheteroaryl- (i.e., heterocycloalkylfusedheteroaryl-);

R⁶ is preferably selected from the group consisting of H, alkyl, alkynyl, cycloalkyl, cycloalkenyl, heterocycloalkyl, aryl, heteroaryl, heteroarylalkyl and —C(═NOR¹⁵)R¹⁶, and more preferably selected from the group consisting of H, alkyl, cycloalkyl, heterocycloalkyl, aryl, heteroaryl and heteroarylalkyl, and most preferably selected from the group consisting of H, alkyl, cycloalkyl, heterocycloalkyl, aryl and heteroaryl;

R² preferably represents H, —S(O)R⁴, —S(O)₂R⁴, —S(O)NHR⁴, —S(O)N(R⁴)₂, —S(O)NH₂, —S(O)₂NHR⁴, —S(O)₂N(R⁴)₂, —S(O)₂NH₂, —CN, —C(O)OR⁴, —C(O)NHR⁴, —C(O)N(R⁴)₂, —C(O)NH₂, —C(O)R⁴, unsubstituted alkyl, substituted alkyl, unsubstituted cycloalkyl or substituted cycloalkyl, and more preferably, R² is selected from H, —S(O)₂R⁴, —CN, —C(O)OR⁴, —C(O)R⁴, unsubstituted alkyl or substituted alkyl, and most preferably R² is selected from H, —C(O)R⁴, unsubstituted alkyl or substituted alkyl;

R²¹ is preferably selected from alkyl, alkynyl, cycloalkyl, cycloalkylalkyl, cycloalkenyl, heterocycloalkyl, ═O, ═N—R², aryl, heteroaryl, heteroarylalkyl, halo, —CN, —OR¹⁵, —C(O)R¹⁵, —C(O)OR¹⁵, —C(O)N(R¹⁵)(R¹⁶), —P(O)(CH₃)₂, —SFₛ, —OSF₅ or —Si(R^{15A})₃ (wherein each R^{15A} is independently selected), —S(O)₂N(R¹⁵)(R¹⁶), —C(═NOR¹⁵)R¹⁶, —P(O)(OR¹⁵)(OR¹⁶), —N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —S(O)₂R^{15A} or —O—N═C(R⁴)₂ (wherein each R⁴ is independently selected), and more preferably R²¹ is selected from alkyl, cycloalkyl, heterocycloalkyl, ═O, ═N—R², aryl, heteroaryl, halo, —CN, —OR¹⁵, —C(O)R¹⁵, —C(O)N(R¹⁵)(R¹⁶), —SF₅, —OSF₅, —Si(R^{15A})₃ (wherein each R^{15A} is independently selected), —S(O)₂N(R¹⁵)(R¹⁶), —C(═NOR¹⁵)R¹⁶, —N(R¹⁵)(R¹⁶), —N(R¹⁵)C(O)R¹⁶, —N(R¹⁵)S(O)₂R^{16A}, —N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —S(O)₂R^{15A} pr —O—N═C(R⁴)₂ (wherein each R⁴ is independently selected), and most preferably R²¹ is selected from alkyl, cycloalkyl, heterocycloalkyl, ═O, ═N—R², aryl, heteroaryl, halo, —CN, —OR¹⁵, —C(O)N(R¹⁵)(R¹⁶), —SF₅, —OSF₅, —Si(R^{15A})₃ (wherein each R^{15A} is independently selected), C(═NOR⁵)R¹⁶, —N(R¹⁵)(R¹⁶), —N(R¹⁵)C(O)R¹⁶, —N(R¹⁵)C(O)N(R¹⁶)(R¹⁷), —N(R¹⁵)C(O)OR¹⁶, —S(O)R^{15A}, ═NOR⁵, —S(O)₂R^{15A} or —O—N═C(R⁴)₂ (wherein each R⁴ is independently selected);

G is preferably selected from the group consisting of: —C(O)—, —CH₂—, CF₂, —CH═CH—, —C═C—, —C(O)NR⁵—, —NR⁵C(O)—, —(C═NR²)—, —S(O)ₜNR⁵—, —NR⁵S(O)ₜ—, —NH—, —O—, —S—, —S(O)—, —S(O)₂— and a direct bond, and R⁹ is a 5-membered ring heteroaryl group, and R¹⁰ is aryl or a 6-membered ring heteroaryl group, and more preferably the group R⁹— R¹⁰-G- represents in each formula

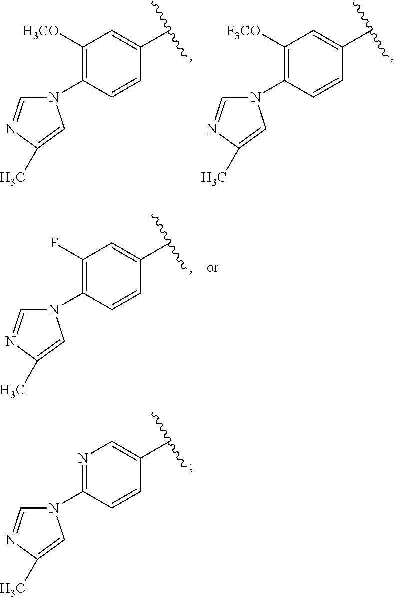

and most preferably, in each formula the group R⁹— R¹⁰-G- represents in each formula

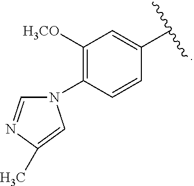

Another embodiment of this invention is directed to a pharmaceutically acceptable salt of a compound of formulas 1 through 4, 1.0 through 11.0, 1.1 through 11.1 herein.

Another embodiment of this invention is directed to a pharmaceutically acceptable ester of a compound of formulas 1 through 4, 1.0 through 11.0, 1.1 through 11.1 herein.

Another embodiment of this invention is directed to a solvate of a compound of formulas 1 through 4, 1.0 through 11.0, 1.1 through 11.1 herein.

Another embodiment of this invention is directed to a compound of Formula I in pure and isolated form. And in one example the compound of Formula I is selected from of a compound of formulas 1 through 4, 1.0 through 11.0, 1.1 through 11.1 herein.

Another embodiment of this invention is directed to a compound of Formula I in pure form. And in one example the compound of Formula I is selected from of a compound of formulas 1 through 4, 1.0 through 11.0, 1.1 through 11.1 herein.

Another embodiment of this invention is directed to a compound of Formula I in isolated form. And in one example the compound of Formula I is selected from of a compound of formulas 1 through 4, 1.0 through 11.0, 1.1 through 11.1 herein.

Another embodiment of this invention is directed to a compound of Formula I selected from of a compound of formulas 1 through 4, 1.0 through 11.0, 1.1 through 11.1 herein.

Another embodiment of this invention is directed to a pharmaceutical composition comprising a therapeutically effective amount of at least one compound of Formula I, or a pharmaceutically acceptable salt, solvate, or ester thereof, and at least one pharmaceutically acceptable carrier.

Another embodiment of this invention is directed to a pharmaceutical composition comprising an effective amount of one or more (e.g., one) compounds of Formula I and a pharmaceutically acceptable carrier.

Another embodiment of this invention is directed to a pharmaceutical composition comprising an effective amount of a pharmaceutically acceptable salt of one or more (e.g., one) compounds of Formula I and a pharmaceutically acceptable carrier.

Another embodiment of this invention is directed to a pharmaceutical composition comprising an effective amount of a pharmaceutically acceptable ester of one or more (e.g., one) compounds of Formula I and a pharmaceutically acceptable carrier.

Another embodiment of this invention is directed to a pharmaceutical composition comprising an effective amount of a solvate of one or more (e.g., one) compounds of Formula I and a pharmaceutically acceptable carrier.

Another embodiment of this invention is directed to a pharmaceutical composition comprising an effective amount of one or more (e.g., one) compounds of Formula I, and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of at least one compound of Formula I, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds of Formula I, and effective amount of one or more BACE inhibitors, and a pharmaceutically acceptable carrier.

Another embodiment of this invention is directed to a pharmaceutical composition comprising an effective amount of one or more (e.g., one) compounds of Formula I, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of Formula I, and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds of Formula I, and effective amount of Exelon (rivastigmine), and a pharmaceutically acceptable carrier.

Another embodiment of this invention is directed to a pharmaceutical composition comprising an effective amount of one or more (e.g., one) compounds of Formula I, and effective amount of Cognex (tacrine), and a pharmaceutically acceptable carrier.

Another embodiment of this invention is directed to a pharmaceutical composition comprising an effective amount of one or more (e.g., one) compounds of Formula I, and effective amount of a Tau kinase inhibitor, and a pharmaceutically acceptable carrier.

Another embodiment of this invention is directed to a pharmaceutical composition comprising an effective amount of one or more (e.g., one) compounds of Formula I, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of Formula I, and effective amount of one anti-Abeta vaccine (active immunization), and a pharmaceutically acceptable carrier.

Another embodiment of this invention is directed to a pharmaceutical composition comprising an effective amount of one or more (e.g., one) compounds of Formula I, and effective amount of one or more APP ligands, and a pharmaceutically acceptable carrier.

Another embodiment of this invention is directed to a pharmaceutical composition comprising an effective amount of one or more (e.g., one) compounds of Formula I, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of Formula I, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of Formula I, and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds of Formula I, and effective amount of one or more LXR agonists, and a pharmaceutically acceptable carrier.

Another embodiment of this invention is directed to a pharmaceutical composition comprising an effective amount of one or more (e.g., one) compounds of Formula I, and effective amount of one or more LRP mimics, and a pharmaceutically acceptable carrier.

Another embodiment of this invention is directed to a pharmaceutical composition comprising an effective amount of one or more (e.g., one) compounds of Formula I, and effective amount of one or more 5-HT6 receptor antagonists, and a pharmaceutically acceptable carrier.

Another embodiment of this invention is directed to a pharmaceutical composition comprising an effective amount of one or more (e.g., one) compounds of Formula I, and effective amount of one or more nicotinic receptor agonists, and a pharmaceutically acceptable carrier.

Another embodiment of this invention is directed to a pharmaceutical composition comprising an effective amount of one or more (e.g., one) compounds of Formula I, and effective amount of one or more H3 receptor antagonists, and a pharmaceutically acceptable carrier.

Another embodiment of this invention is directed to a pharmaceutical composition comprising an effective amount of one or more (e.g., one) compounds of Formula I, and effective amount of one or more histone deacetylase inhibitors, and a pharmaceutically acceptable carrier.

Another embodiment of this invention is directed to a pharmaceutical composition comprising an effective amount of one or more (e.g., one) compounds of Formula I, and effective amount of one or more hsp90 inhibitors, and a pharmaceutically acceptable carrier.

Another embodiment of this invention is directed to a pharmaceutical composition comprising an effective amount of one or more (e.g., one) compounds of Formula I, and effective amount of one or more ml muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of Formula I, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of Formula I, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of Formula I, and effective amount of one or more one mGluR2/3 antagonists, and a pharmaceutically acceptable carrier.

Another embodiment of this invention is directed to a pharmaceutical composition comprising an effective amount of one or more (e.g., one) compounds of Formula I, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of Formula I, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of Formula I, and effective amount of one or more PAl-1 inhibitors, and a pharmaceutically acceptable carrier.

Another embodiment of this invention is directed to a pharmaceutical composition comprising an effective amount of one or more (e.g., one) compounds of Formula I, and effective amount of one or more agents that can induce Abeta efflux such as gelsolin, and a pharmaceutically acceptable carrier.

Other embodiments of this invention are directed to any one of the above embodiments directed to pharmaceutical compositions wherein the compound of Formula I is selected from the group consisting of a compound of formulas 1.0 through 14.0, 1.1 through 14.1 and 1.2, 2.2 and 3.2 herein.

The compounds of Formula I can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

Another embodiment of this invention is directed to a method of treating a central nervous system disorder comprising administering a therapeutically effective amount of at least one compound of Formula I to a patient in need of such treatment.

Another embodiment of this invention is directed to a method of treating a central nervous system disorder comprising administering a therapeutically effective amount of a pharmaceutical composition comprising a therapeutically effective amount of at least one compound of Formula I, or a pharmaceutically acceptable salt, solvate, or ester thereof, and at least one pharmaceutically acceptable carrier.

Another embodiment of this invention is directed to a method of treating a central nervous system disorder comprising administering a therapeutically effective amount of a pharmaceutical composition comprising a therapeutically effective amount of at least one compound of Formula I, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a method for modulating (including inhibiting, antagonizing and the like) gamma-secretase comprising administering an effective amount of one or more (e.g., one) compounds of Formula I to a patient in need of such treatment.

Another embodiment of this invention is directed to a method for modulating (including inhibiting, antagonizing and the like) gamma-secretase, comprising administering an effective amount of a compound of Formula I to a patient in need of treatment.

Another embodiment of this invention is directed to a method of treating one or more neurodegenerative diseases, comprising administering an effective amount of one or more (e.g., one) compounds of Formula I to a patient in need of treatment.

Another embodiment of this invention is directed to a method of treating one or more neurodegenerative diseases, comprising administering an effective amount of a compound of Formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds of Formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound of Formula I to a patient in need of treatment.

Another embodiment of this invention is directed to a method of treating Alzheimer's disease, comprising administering an effective amount of one or more (e.g., one) compounds of Formula I to a patient in need of treatment.

Another embodiment of this invention is directed to a method of treating Alzheimer's disease, comprising administering an effective amount of a compound of Formula I to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of Formula I to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of Formula I to a patient in need of treatment.

Another embodiment of this invention is directed to a method of treating mild cognitive impairment, comprising administering an effective amount of one or more (e.g., one) compounds of Formula I to a patient in need of treatment.

Another embodiment of this invention is directed to a method of treating glaucoma, comprising administering an effective amount of one or more (e.g., one) compounds of Formula I to a patient in need of treatment.

Another embodiment of this invention is directed to a method of treating cerebral amyloid angiopathy, comprising administering an effective amount of one or more (e.g., one) compounds of Formula I to a patient in need of treatment.

Another embodiment of this invention is directed to a method of treating stroke, comprising administering an effective amount of one or more (e.g., one) compounds of Formula I to a patient in need of treatment.

Another embodiment of this invention is directed to a method of treating dementia, comprising administering an effective amount of one or more (e.g., one) compounds of Formula I to a patient in need of treatment.

Another embodiment of this invention is directed to a method of treating microgliosis, comprising administering an effective amount of one or more (e.g., one) compounds of Formula I to a patient in need of treatment.

Another embodiment of this invention is directed to a method of treating brain inflammation, comprising administering an effective amount of one or more (e.g., one) compounds of Formula I to a patient in need of treatment.

Another embodiment of this invention is directed to a method of treating olfactory function loss, comprising administering an effective amount of one or more (e.g., one) compounds of Formula I to a patient in need of treatment.

Another embodiment of this invention is directed to a method of treating Downs syndrome, comprising administering an effective amount of one or more (e.g., one) compounds of Formula I to a patient in need of treatment.

Another embodiment of this invention is directed to a method of treating Downs syndrome, comprising administering an effective amount of a compound of Formula I to a patient in need of treatment.

Other embodiments of this invention are directed to any one of the above embodiments directed to methods of treating wherein the compound of Formula I is selected from the group consisting compounds of formulas 1 through 4, 1.0 through 11.0, 1.1 through 11.1 herein.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of Formula I and the administration of an effective amount of one or more (e.g., one) other pharmaceutical active ingredients (e.g., drugs). The compounds of Formula I and the other drugs can be administered separately (i.e., each is in its own separate dosage form), or the compounds of Formula I can be combined with the other drugs in the same dosage form.

Thus, other embodiments of this invention are directed to any one of the methods of treatment, or methods of inhibiting, described herein, wherein an effective amount of the compound of Formula I is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAl-1 inhibitors; and agents that can induce Abeta efflux such as gelsolin.

Another embodiment of this invention is directed to a method of treating Alzheimer's disease, comprising administering an effective amount of one or more (e.g., one) compounds of Formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of Formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds of Formula I, in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds of Formula I, in combination with an effective amount of one or more BACE inhibitors.

Another embodiment of this invention is directed to a method of treating Alzheimer's disease, comprising administering an effective amount of one or more compounds of Formula I, in combination with an effective amount of Exelon (rivastigmine).

Another embodiment of this invention is directed to a method of treating Alzheimer's disease, comprising administering an effective amount of one or more compounds of Formula I, in combination with an effective amount of Cognex (tacrine).

Another embodiment of this invention is directed to a method of treating Alzheimer's disease, comprising administering an effective amount of one or more compounds of Formula I, in combination with an effective amount of a Tau kinase inhibitor.

Another embodiment of this invention is directed to a method of treating Alzheimer's disease, comprising administering an effective amount of one or more compounds of Formula I, in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of Formula I, in combination with an effective amount of one anti-Abeta vaccination (active immunization).

Another embodiment of this invention is directed to a method of treating Alzheimer's disease, comprising administering an effective amount of one or more compounds of Formula I, in combination with an effective amount of one or more APP ligands.

Another embodiment of this invention is directed to a method of treating Alzheimer's disease, comprising administering an effective amount of one or more compounds of Formula I, in combination with an effective amount of one or more agents that upregulate insulin degrading enzyme and/or neprilysin.

Another embodiment of this invention is directed to a method of treating Alzheimer's disease, comprising administering an effective amount of one or more compounds of Formula I, in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of Formula I, in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of Formula I, in combination with an effective amount of one or more LXR agonists.

Another embodiment of this invention is directed to a method of treating Alzheimer's disease, comprising administering an effective amount of one or more compounds of Formula I, in combination with an effective amount of one or more LRP mimics.

Another embodiment of this invention is directed to a method of treating Alzheimer's disease, comprising administering an effective amount of one or more compounds of Formula I, in combination with an effective amount of one or more 5-HT6 receptor antagonists.

Another embodiment of this invention is directed to a method of treating Alzheimer's disease, comprising administering an effective amount of one or more compounds of Formula I, in combination with an effective amount of one or more nicotinic receptor agonists.

Another embodiment of this invention is directed to a method of treating Alzheimer's disease, comprising administering an effective amount of one or more compounds of Formula I, in combination with an effective amount of one or more H3 receptor antagonists.

This invention also provides a method of treating Alzheimer's disease, comprising administering an effective amount of one or more compounds of Formula I, in combination with an effective amount of one or more histone deacetylase inhibitors.

Another embodiment of this invention is directed to a method of treating Alzheimer's disease, comprising administering an effective amount of one or more compounds of Formula I, in combination with an effective amount of one or more hsp90 inhibitors.

Another embodiment of this invention is directed to a method of treating Alzheimer's disease, comprising administering an effective amount of one or more compounds of Formula I, in combination with an effective amount of one or more ml muscarinic receptor agonists.

Another embodiment of this invention is directed to a method of treating Alzheimer's disease, comprising administering an effective amount of one or more compounds of Formula I, in combination with an effective amount of one or more 5-HT6 receptor antagonists mGluR1 or mGluR5 positive allosteric modulators or agonists

Another embodiment of this invention is directed to a method of treating Alzheimer's disease, comprising administering an effective amount of one or more compounds of Formula I, in combination with an effective amount of one or more mGluR2/3 antagonists.

Another embodiment of this invention is directed to a method of treating Alzheimer's disease, comprising administering an effective amount of one or more compounds of Formula I, in combination with an effective amount of one or more anti-inflammatory agents that can reduce neuroinflammation.

Another embodiment of this invention is directed to a method of treating Alzheimer's disease, comprising administering an effective amount of one or more compounds of Formula I, in combination with an effective amount of one or more Prostaglandin EP2 receptor antagonists.

Another embodiment of this invention is directed to a method of treating Alzheimer's disease, comprising administering an effective amount of one or more compounds of Formula I, in combination with an effective amount of one or more PAI-1 inhibitors.

Another embodiment of this invention is directed to a method of treating Alzheimer's disease, comprising administering an effective amount of one or more compounds of Formula I, in combination with an effective amount of one or more agents that can induce Abeta efflux such as gelsolin.

Another embodiment of this invention is directed to a method of treating Downs syndrome, comprising administering an effective amount of one or more (e.g., one) compounds of Formula I, in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective amount of a compound of Formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Other embodiments of this invention are directed to any one of the above embodiments directed to combination therapies (i.e., the above methods of treating wherein compounds of Formula I are used in combination with other pharmaceutically active ingredients, i.e., drugs) wherein the compound of Formula I is selected from the group consisting compounds of formulas 1 through 4, 1.0 through 11.0, 1.1 through 11.1 herein.

This invention also provides a kit comprising, in separate containers, in a single package, pharmaceutical compositions for use in combination, wherein one container comprises an effective amount of a compound of Formula I in a pharmaceutically acceptable carrier, and another container (i.e., a second container) comprises an effective amount of another pharmaceutically active ingredient (as described above), the combined quantities of the compound of Formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (l) olfactory function loss.

This invention also provides a kit comprising, in separate containers, in a single package, pharmaceutical compositions for use in combination, wherein one container comprises an effective amount of a compound of Formula I in a pharmaceutically acceptable carrier, and another container (i.e., a second container) comprises an effective amount of another pharmaceutically active ingredient (as described above), the combined quantities of the compound of Formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments directed to kits wherein the compound of Formula I is selected from the group consisting compounds of formulas 1 through 4, 1.0 through 11.0, 1.1 through 11.1 herein.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May, 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

It is noted that the carbons of Formula I and other formulas herein may be replaced with 1 to 3 silicon atoms so long as all valency requirements are satisfied.

As used above, and throughout this disclosure, the following terms, unless otherwise indicated, shall be understood to have the following meanings: “Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

“An effective amount” as used to describe the amount of a compound of Formula I in a pharmaceutical composition, or to describe the amount of a compound of Formula I used in a method of treatment, or to describe the amount of a pharmaceutical composition used in a method of treatment, or to describe the amount of other pharmaceutic ingredients (i.e., drugs) used in a pharmaceutical compositions or methods of treatment, means a therapeutically effective amount.

“Bn” means benzyl.

“Et” means ethyl.

“i-pr” means isopropyl.

“Me” means methyl.

“Pr” means propyl.

“t-Bu” means tert-butyl.

“TBDMSCI” means tert-butyldimethylsilyl chloride.

“DMAP” means 4-(dimethylamino)pyridine.

“Carbocyclic” means a non-aromatic saturated or unsaturated mono- or multicyclic ring system comprising about 3 to about 10 carbon atoms, preferably about to about 10 carbon atoms. Carbocyclic rings include cycloalkyl rings and cycloalkenyl rings as defined below. Thus, examples of carbocyclic rings include bicyclic rings, such as, for example, norbornyl, adamantly, norbornenyl, and

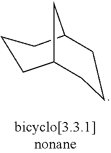

The carbocyclic rings are optionally substituted with one or more independently selected “ring system substituents” as defined below.

“Fused benzocycloalkyl ring” means a phenyl ring fused to a cycloalkyl ring (as cycloalkyl is defined below), such as, for example,

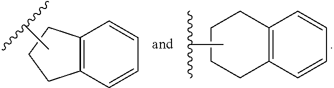

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, ═O, ═N—OY₁, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system.

Examples of such moiety are methylene dioxy, ethylenedioxy, —C(CH₃)₂— and the like which form moieties such as, for example:

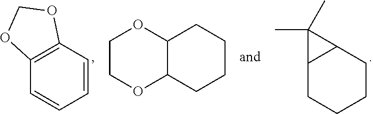

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes rings wherein ═O replaces two available hydrogens on the same carbon atom on a ring system (i.e., heterocyclyl includes rings having a carbonyl in the ring). An example of such moiety is pyrrolidone:

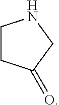

“Heterocyclylalkyl” (or “heterocycloalkylalkyl”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” (or “heterocycloalkenyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom on a ring system (i.e., heterocyclyl includes rings having a carbonyl in the ring). An example of such moiety is pyrrolidinone:

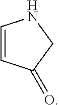

“Heterocyclenylalkyl” (or “heterocycloalkenylalkyl”) means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

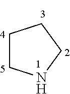

there is no —OH attached directly to carbons marked 2 and 5.

It should also be noted that tautomeric forms such as, for example, the moieties:

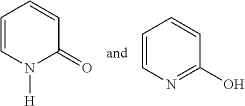

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined.

Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula I or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N-(C₁-C₆)alkylaminoalkyl, —C(Y⁴)Y⁵ wherein Y⁴ is H or methyl and Y⁵ is mono-N- or di-N,N-(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts, solvates, esters and prodrugs thereof, may exist in their tautomeric form (for example, as an amide, enol, keto or imino ether). All such tautomeric forms are contemplated herein as part of the present invention.

The compounds of Formula I may contain asymmetric or chiral centers, and, therefore, exist in different stereoisomeric forms. It is intended that all stereoisomeric forms of the compounds of Formula I as well as mixtures thereof, including racemic mixtures, form part of the present invention. In addition, the present invention embraces all geometric and positional isomers. For example, if a compound of Formula I incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula I may be atropisomers (e.g., substituted biaryls) and are considered as part of this invention. Enantiomers can also be separated by use of chiral HPLC column.

It is also possible that the compounds of Formula I may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula I incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T1/2>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula I, and of the salts, solvates, esters and prodrugs of the compounds of Formula I, are intended to be included in the present invention.

The compounds according to the invention can have pharmacological properties; in particular, the compounds of Formula I can be modulators of gamma secretase (including inhibitors, antagonists and the like).

More specifically, the compounds of Formula I can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula I, or a pharmaceutically acceptable salt, solvate, ester or prodrug of said compound to the mammal.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula I, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Yet another aspect of this invention is a kit comprising an amount of at least one compound of Formula I, or a pharmaceutically acceptable salt, solvate, ester or prodrug of said compound and an amount of at least one additional agent listed above, wherein the amounts of the two or more ingredients result in desired therapeutic effect.

The compounds of the invention can be made by the methodologies illustrated below in which optional double bonds are shown between positions 1-2 and 3-4:

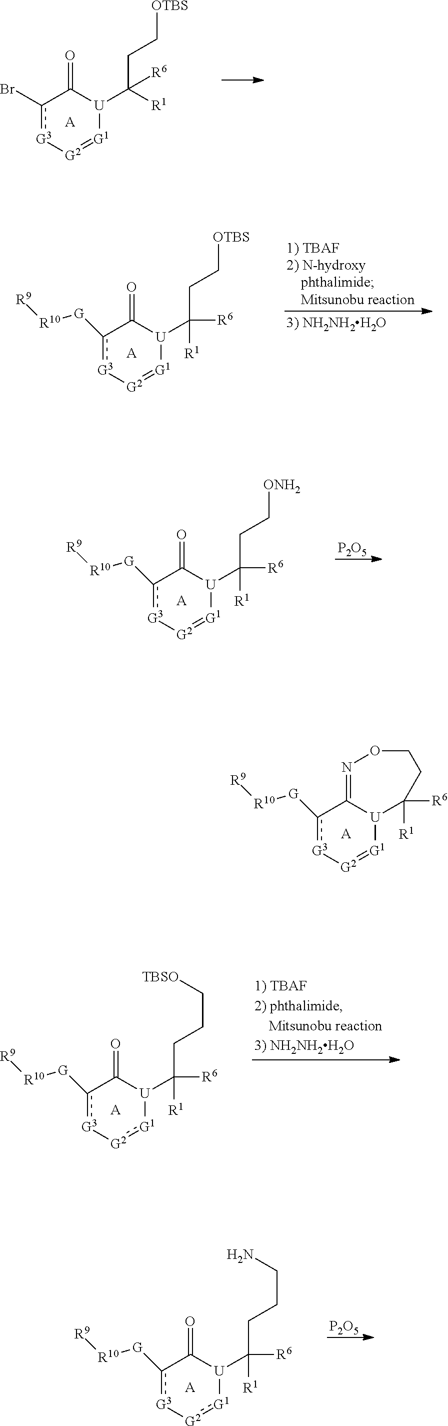

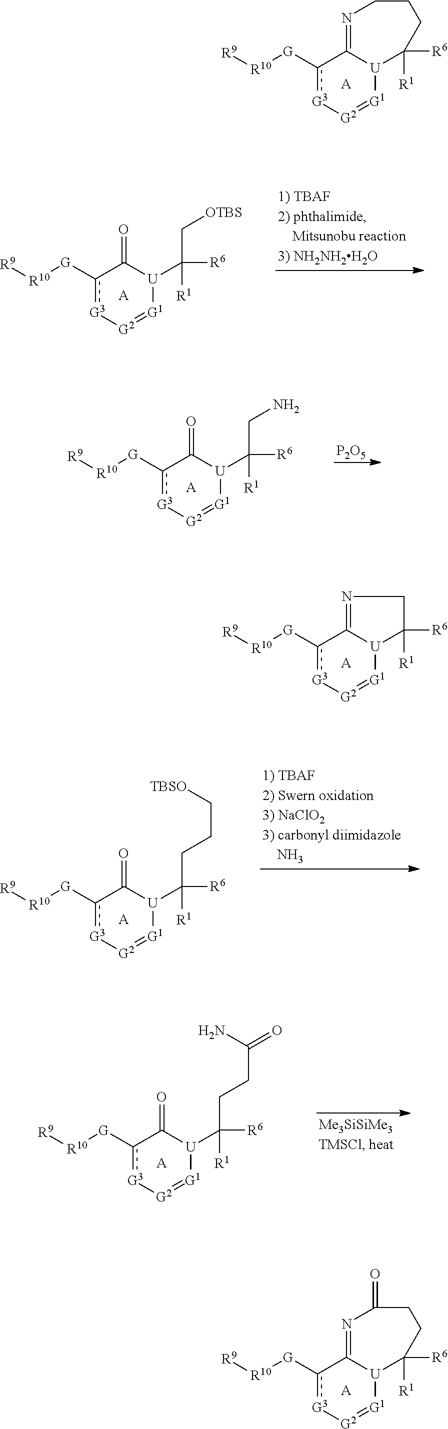

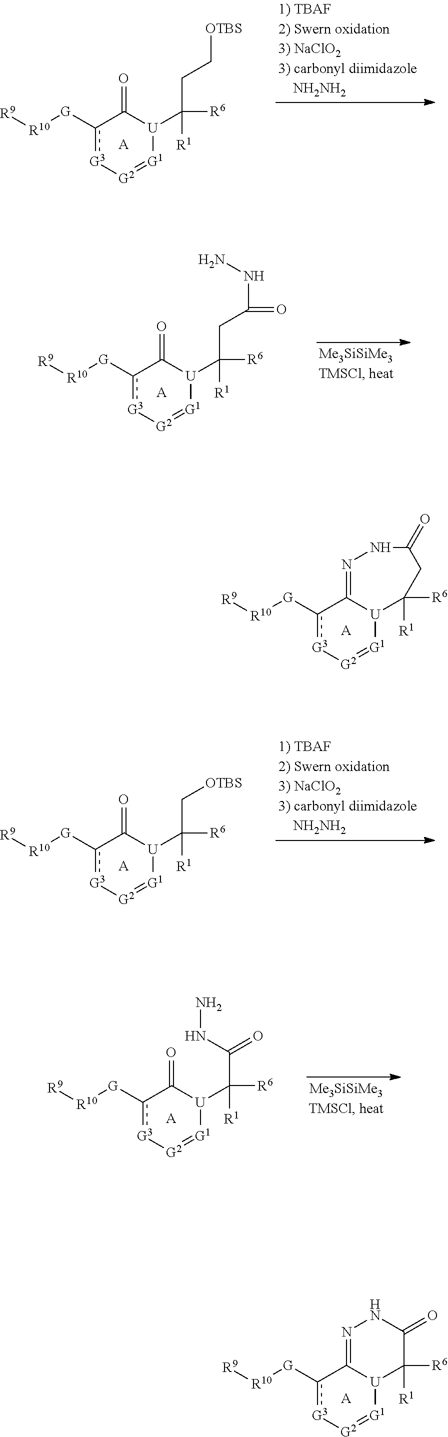

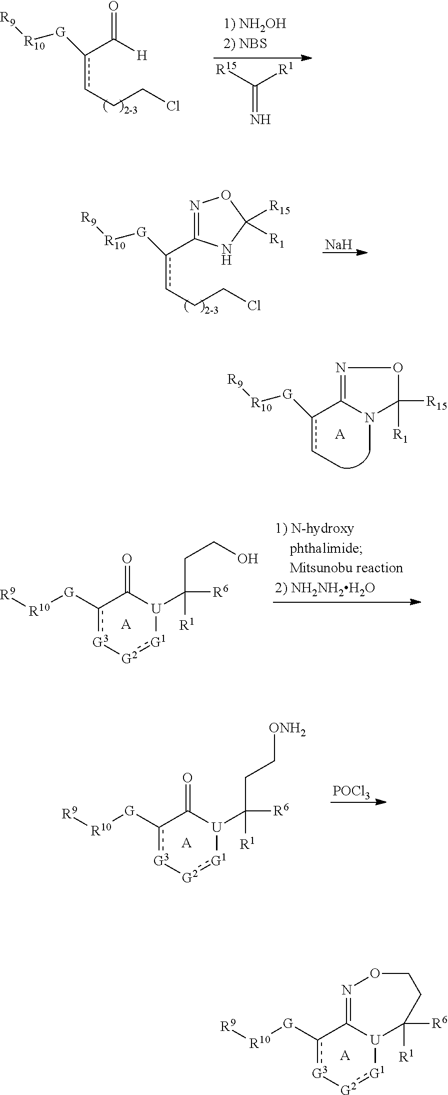

In the following schemes, R=

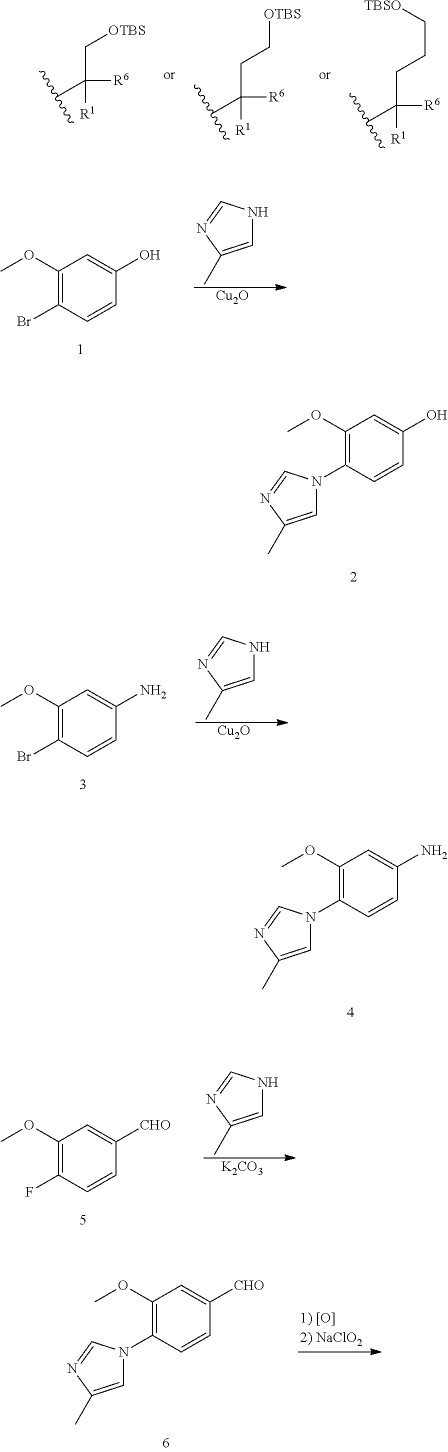

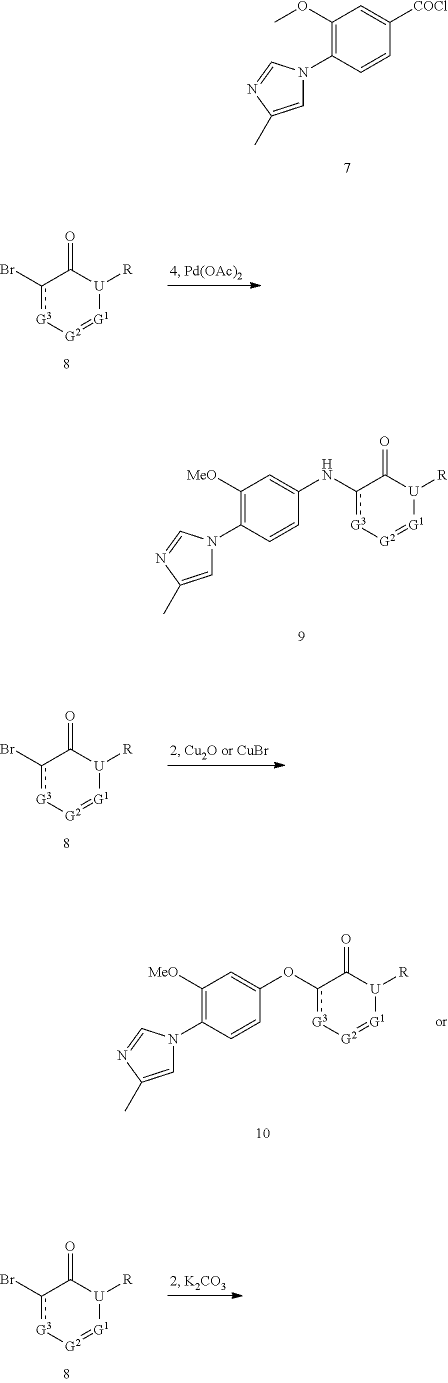

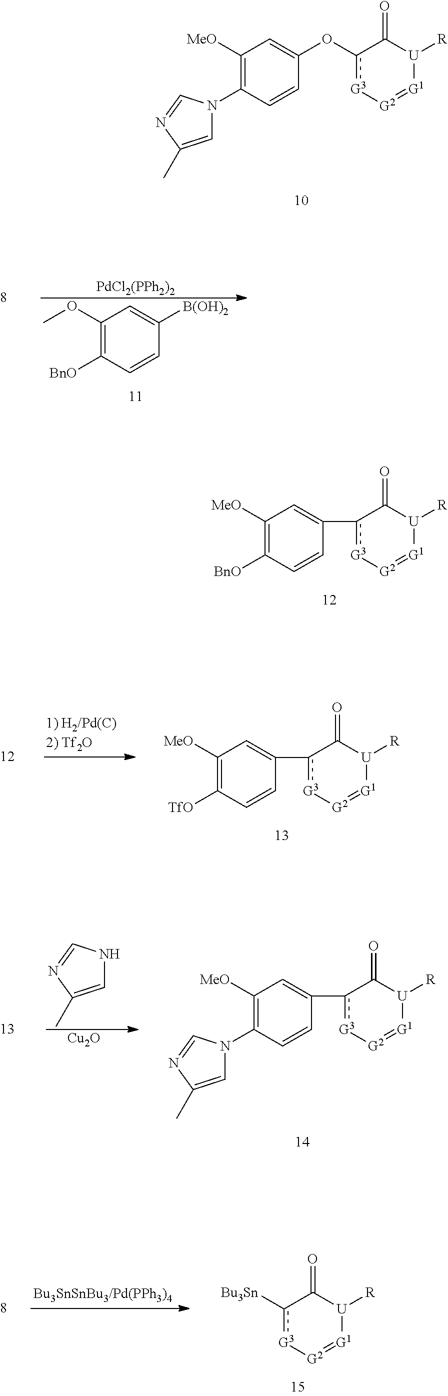

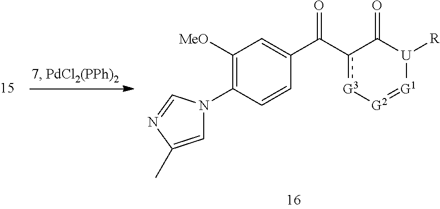

or 15→17→16 as follows:

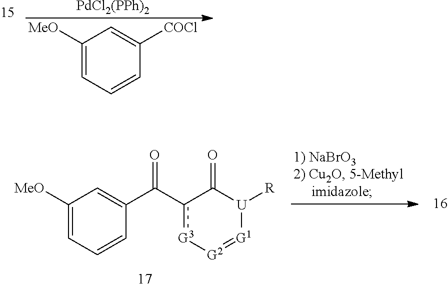

or 8→18→16 as follows:

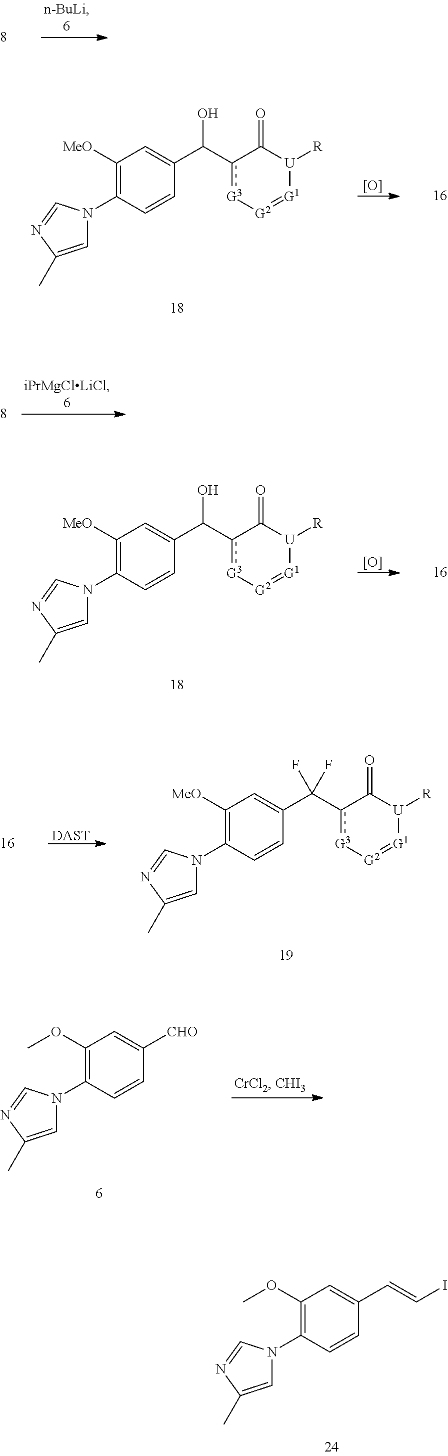

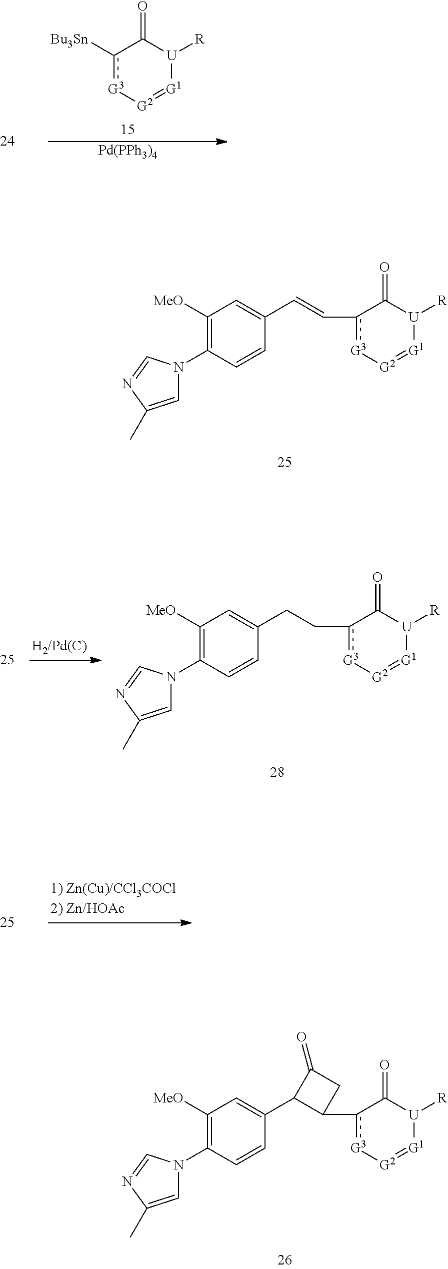

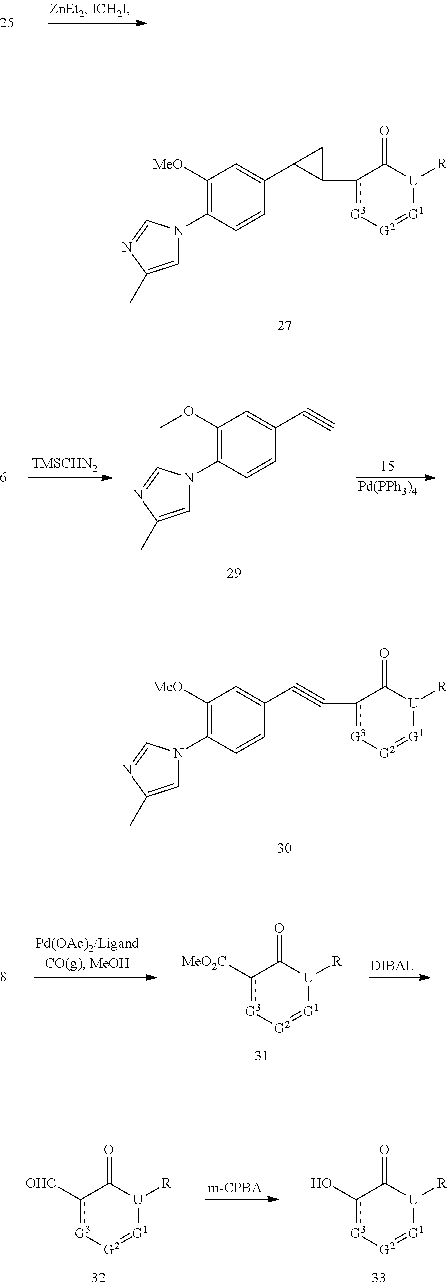

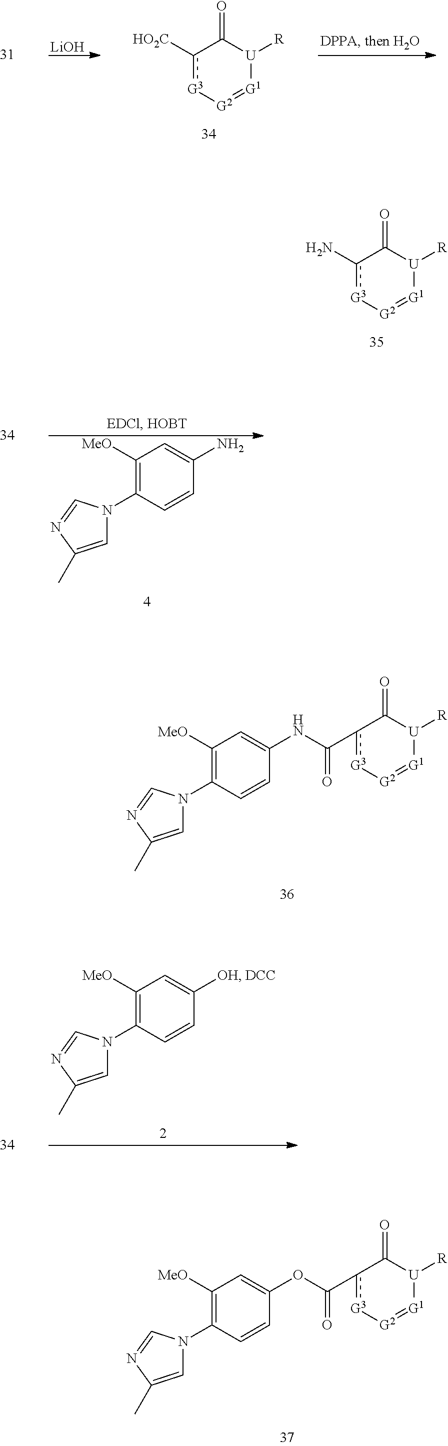

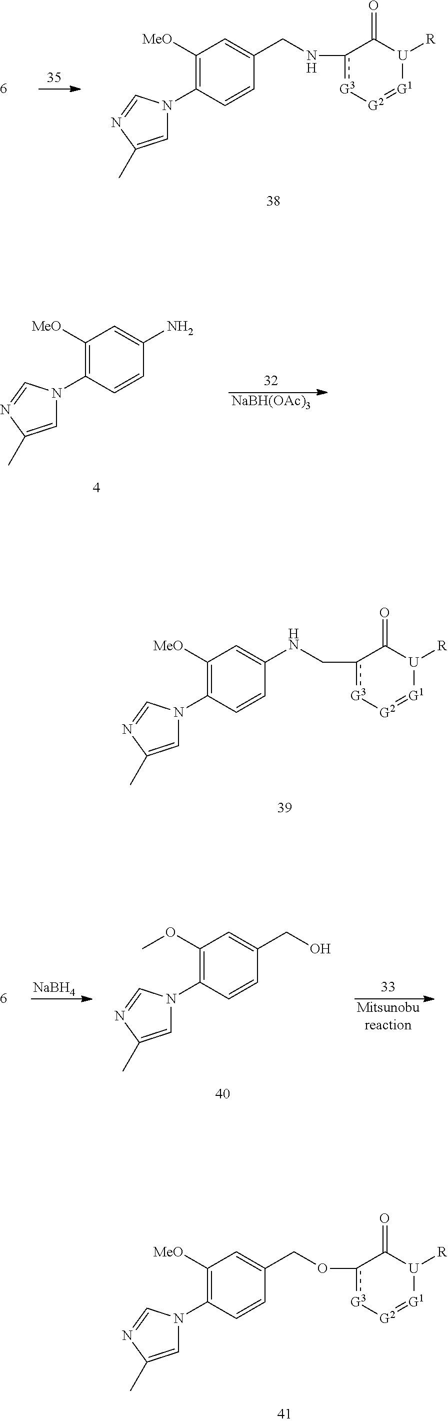

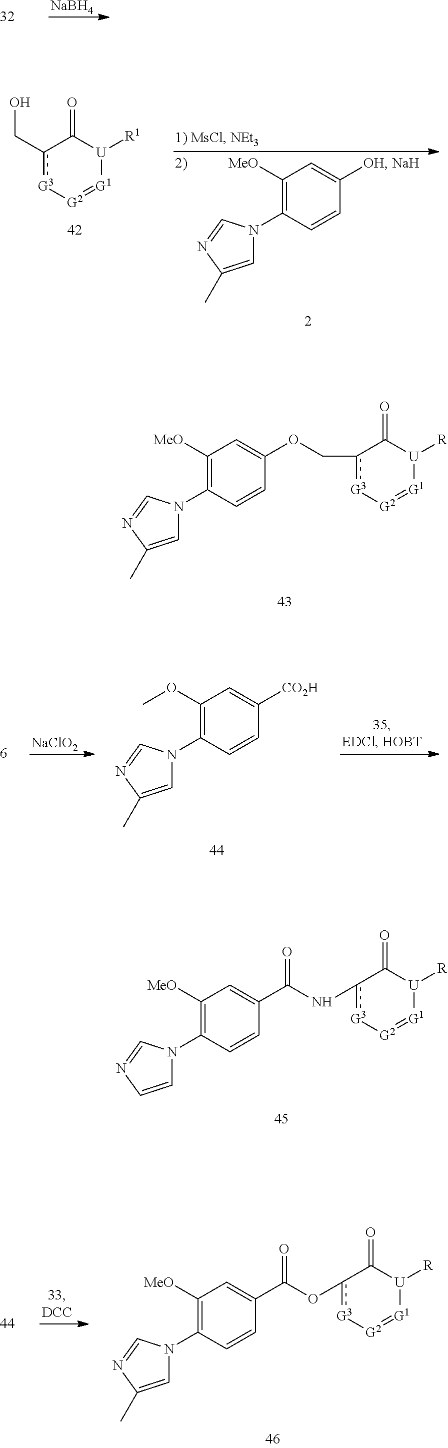

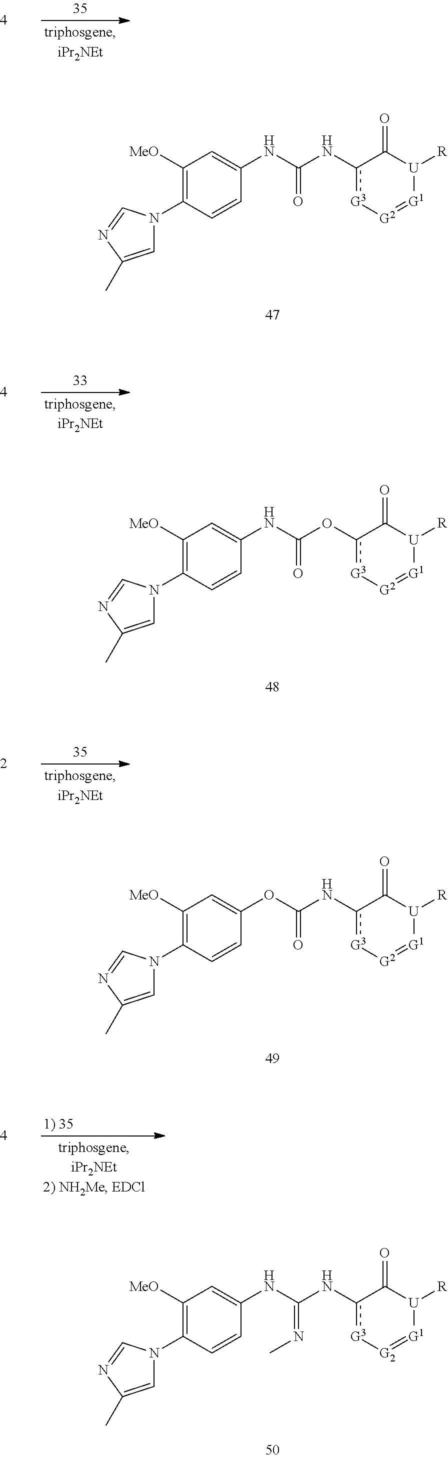

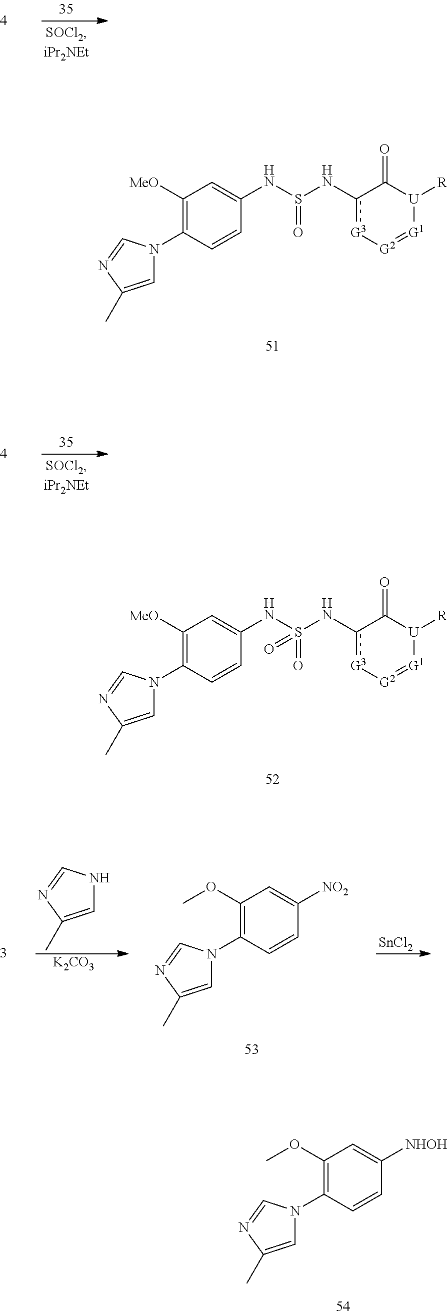

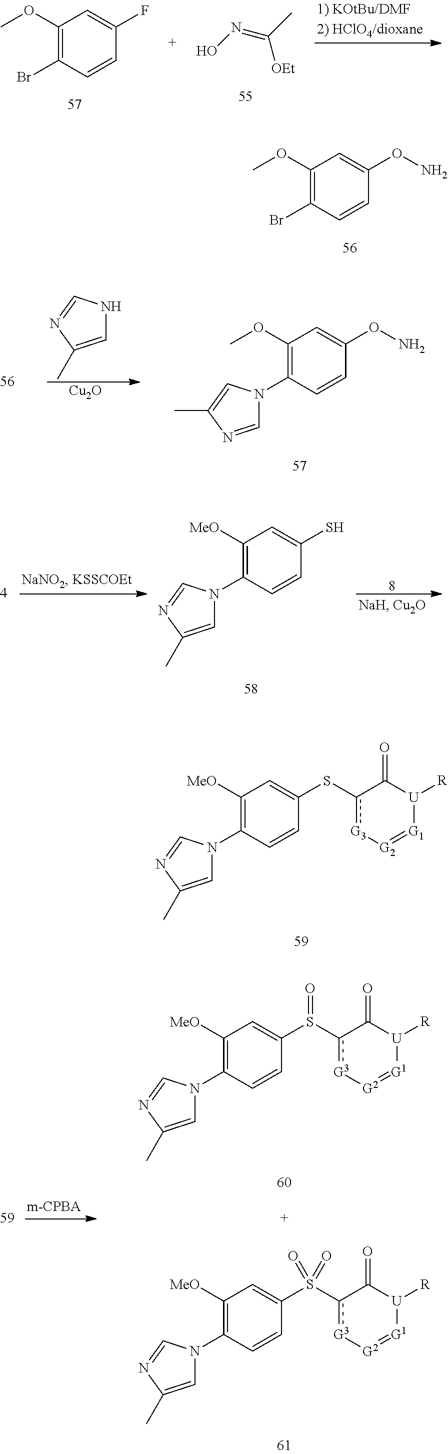

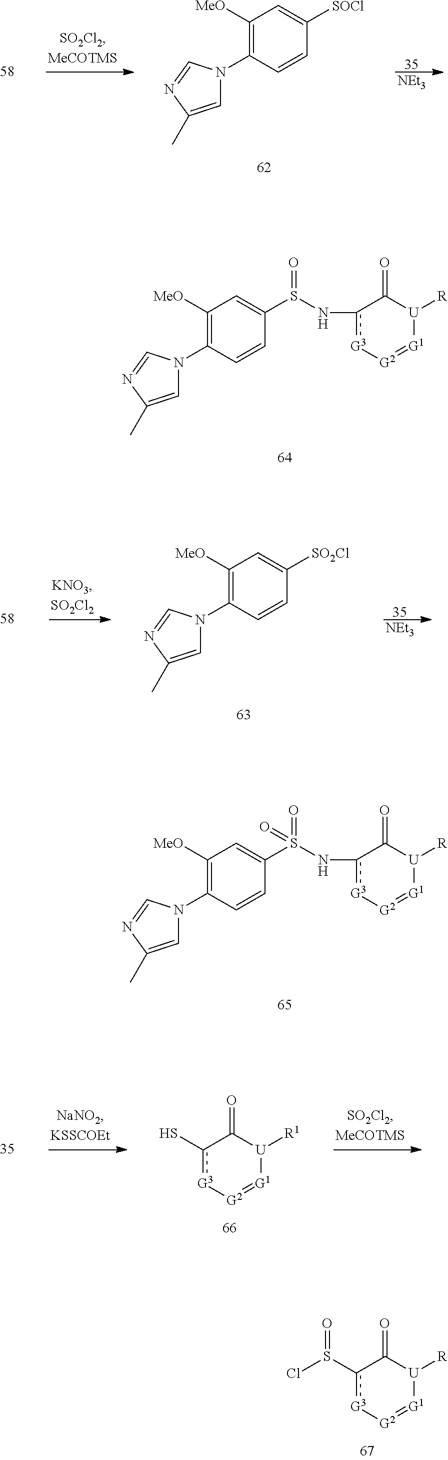

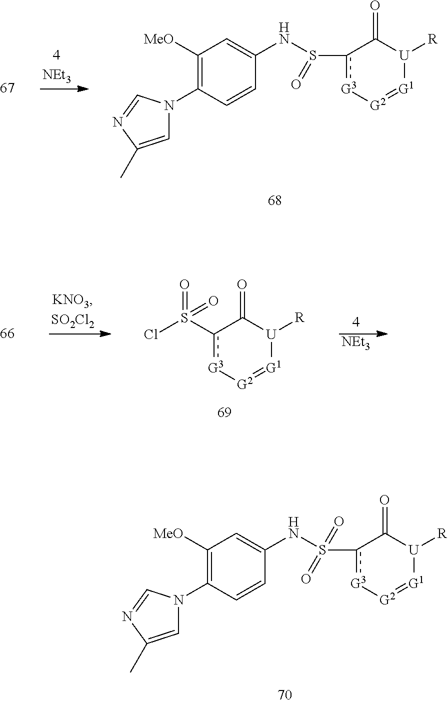

Compounds of the invention having —SF₅ and —Si(R¹⁵)₃ (e.g., —Si(CH₃)₃) groups can be prepared according to the scheme below and by techniques well known in the art. Those skilled in the art will appreciate that any carbon substitutable with a —CF₃ group can be substituted with a —SF₅ or a —Si(R¹⁵)³ (e.g., —Si(CH₃)₃) group using techniques well known in the art.

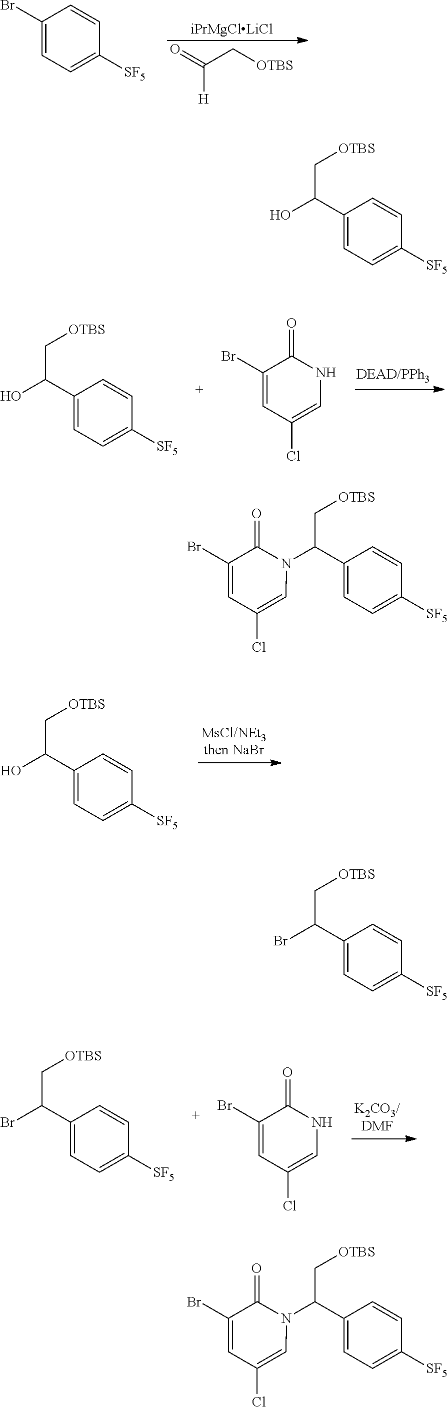

a-e can be prepared in a
similar manner:

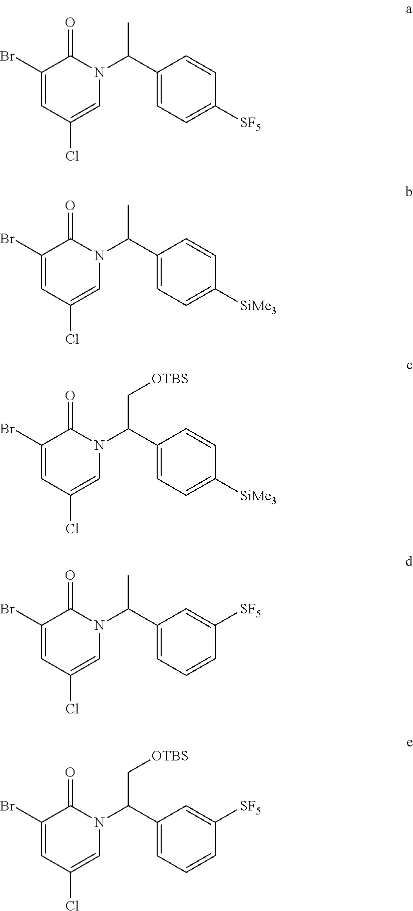

Compounds of the invention having —OSF₅ groups can be prepared according to the scheme below and by techniques well known in the art.

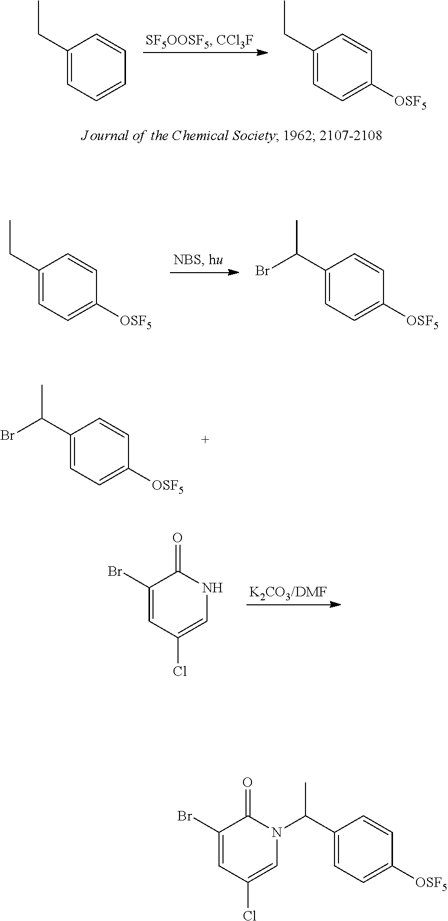

f-h can be prepared in a similar manner:

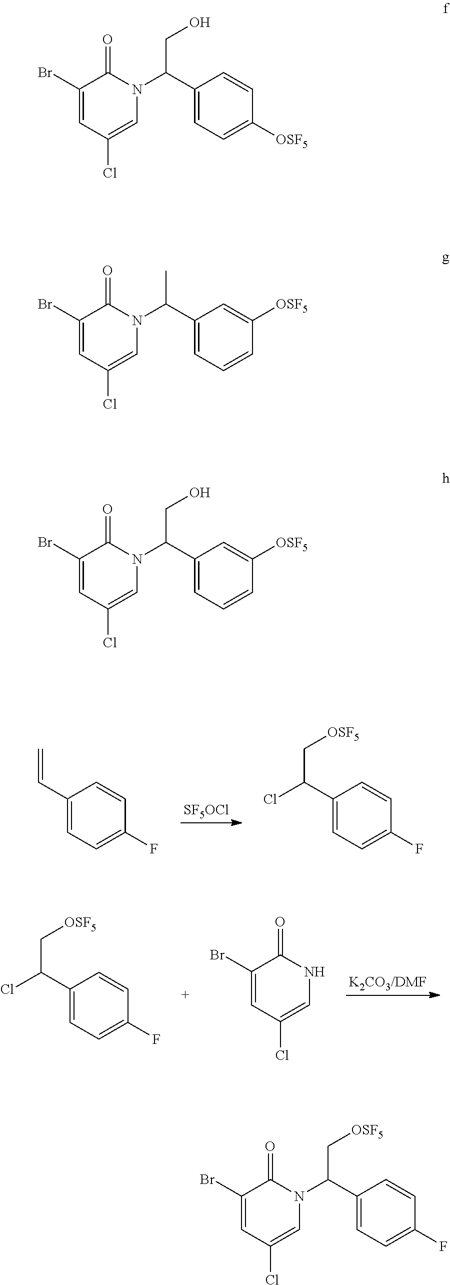

The brominated starting compounds, e.g., compound 8, are commercially available, or can be prepared according to procedures know in the art and the reactions below.

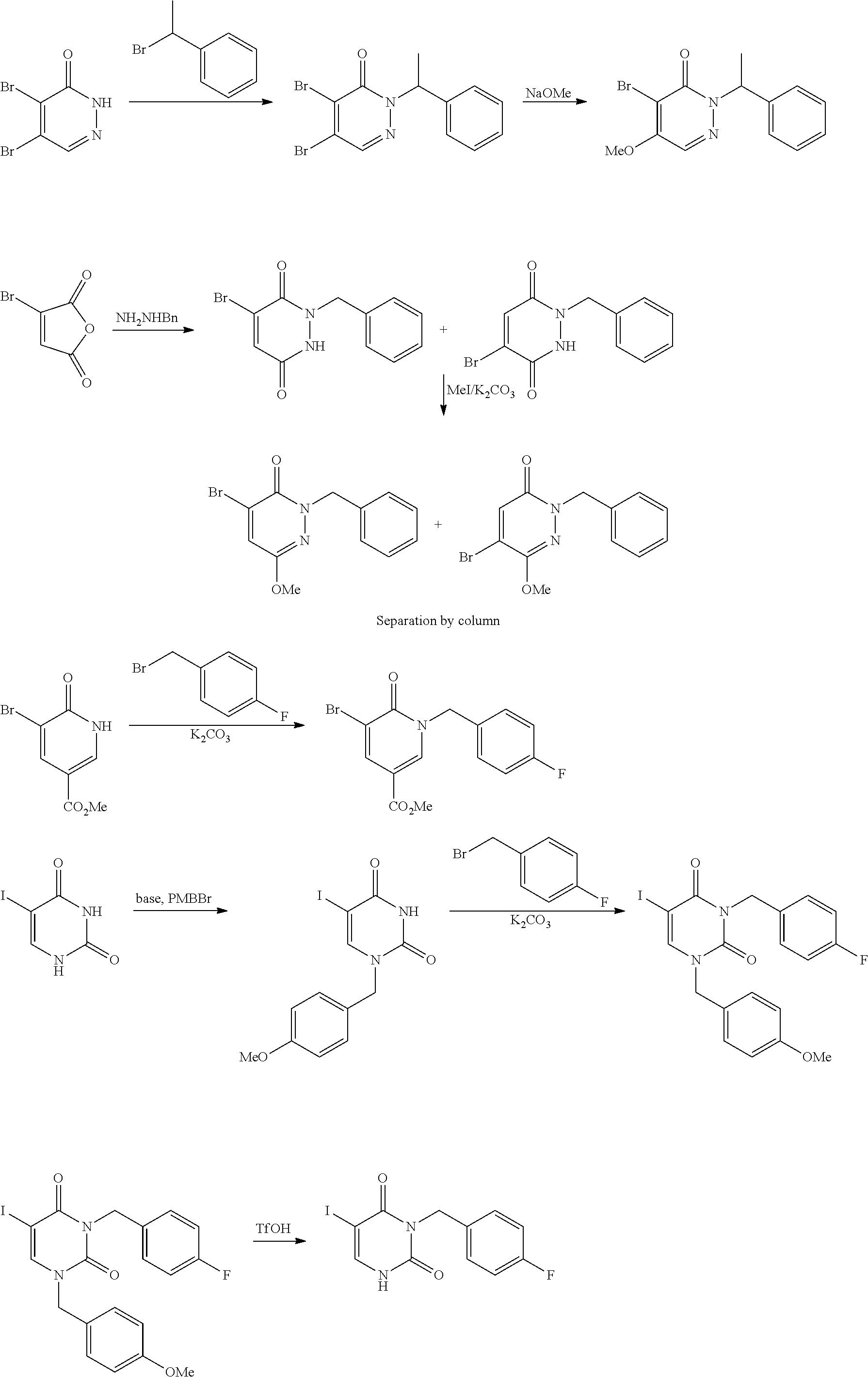

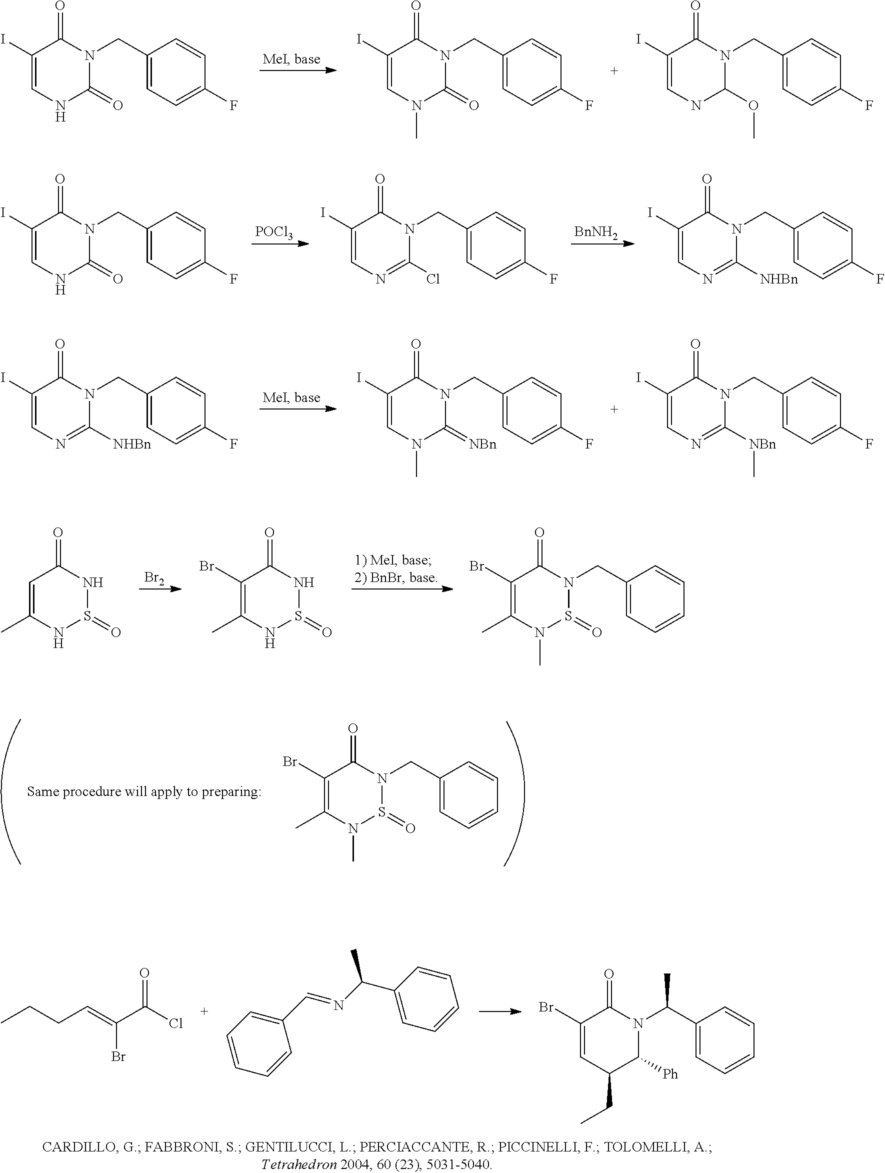

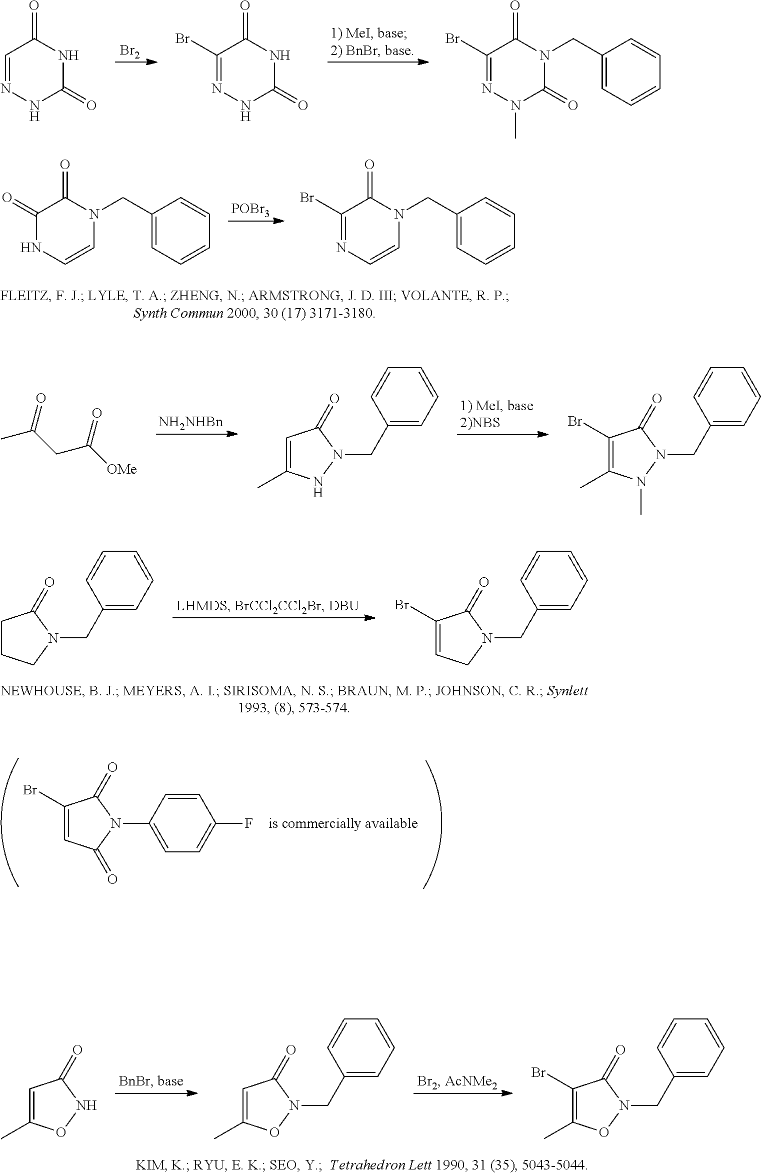

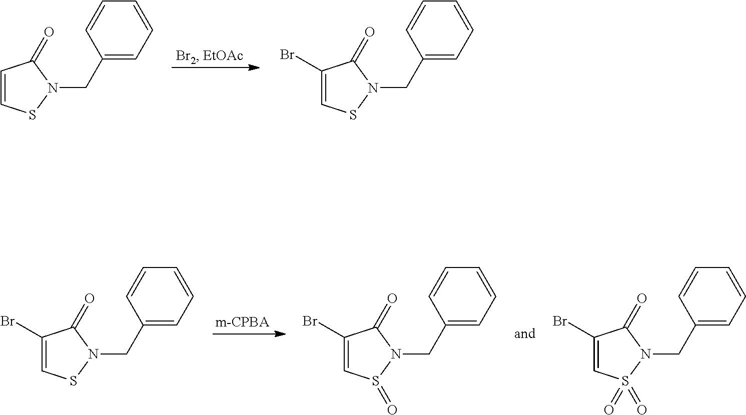

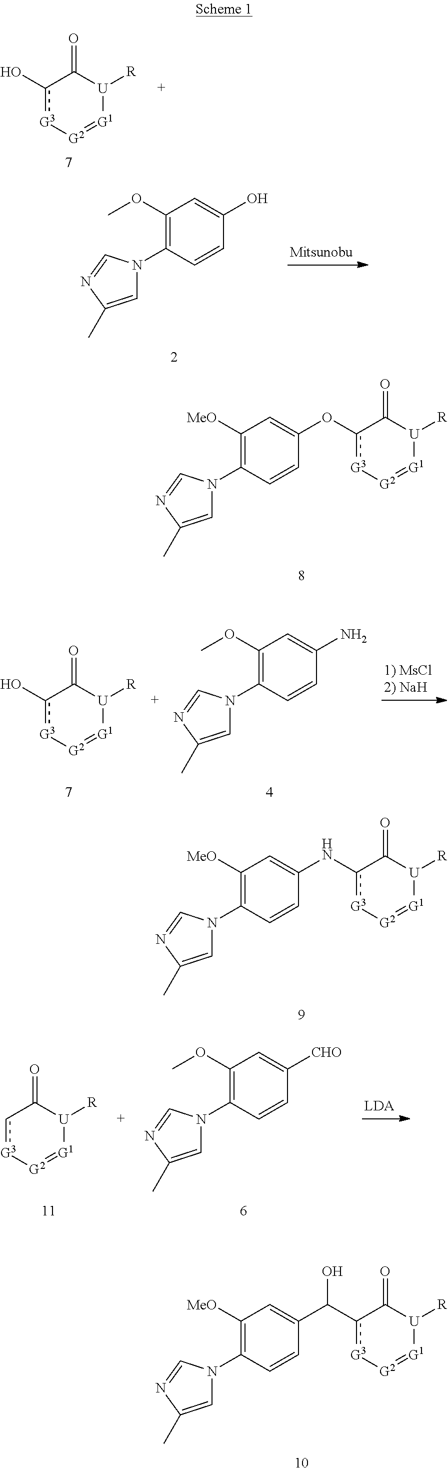

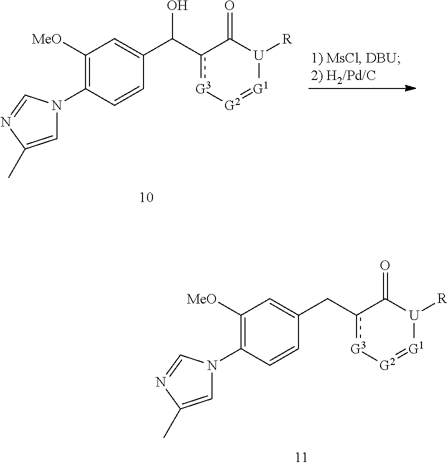

### Preparation of Intermediates

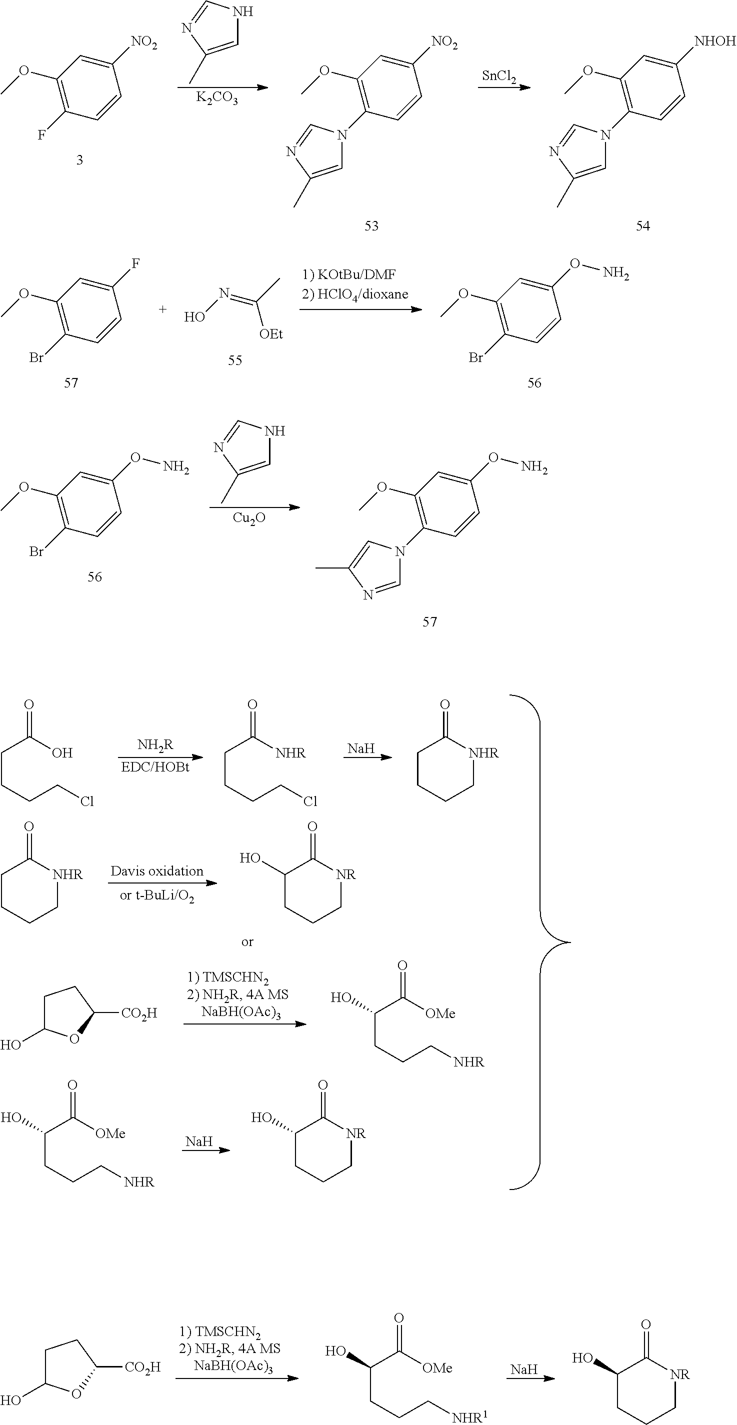

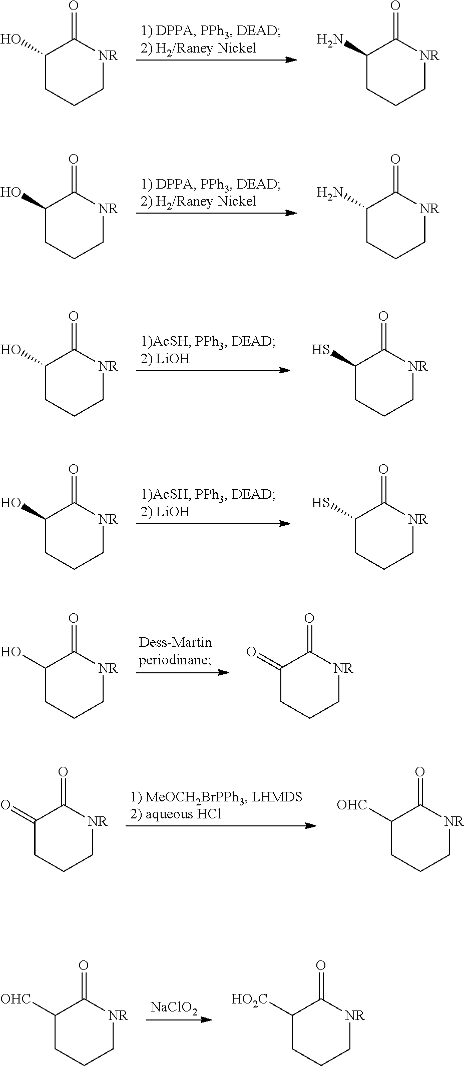

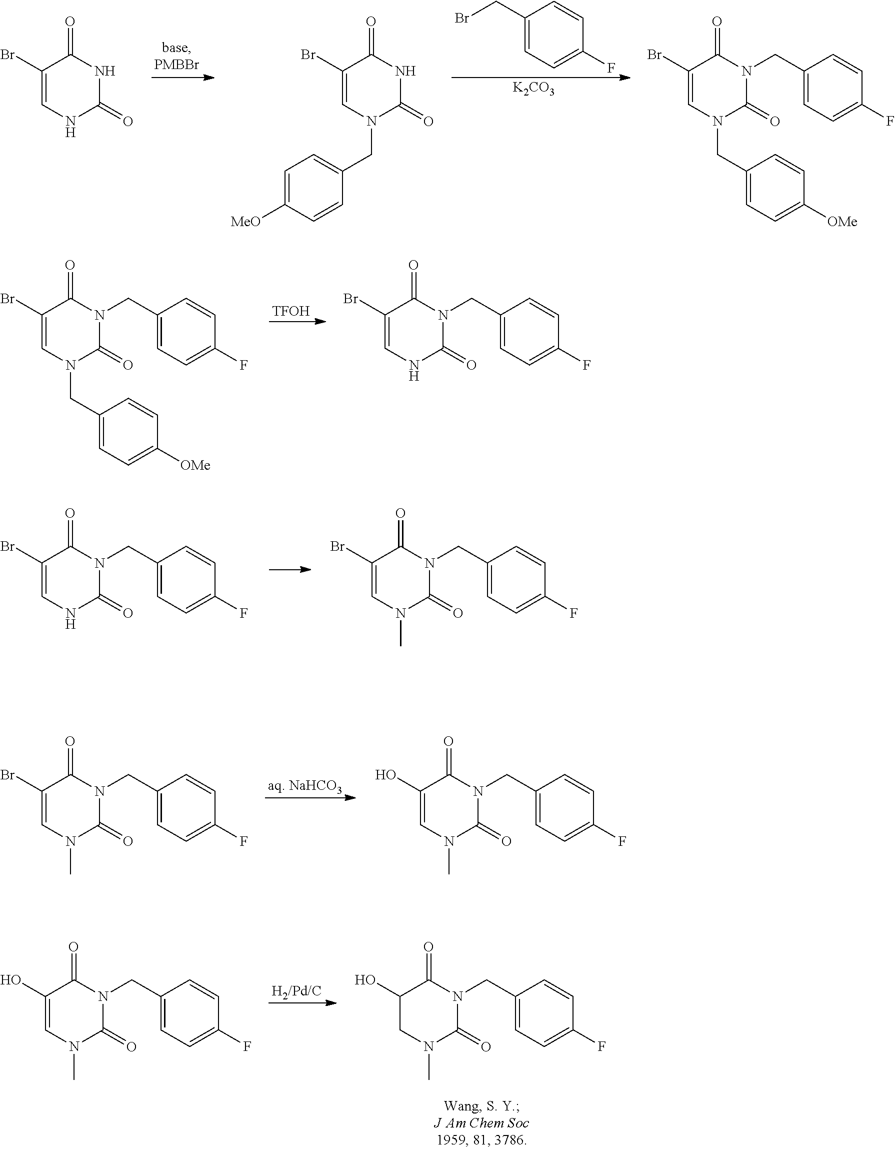

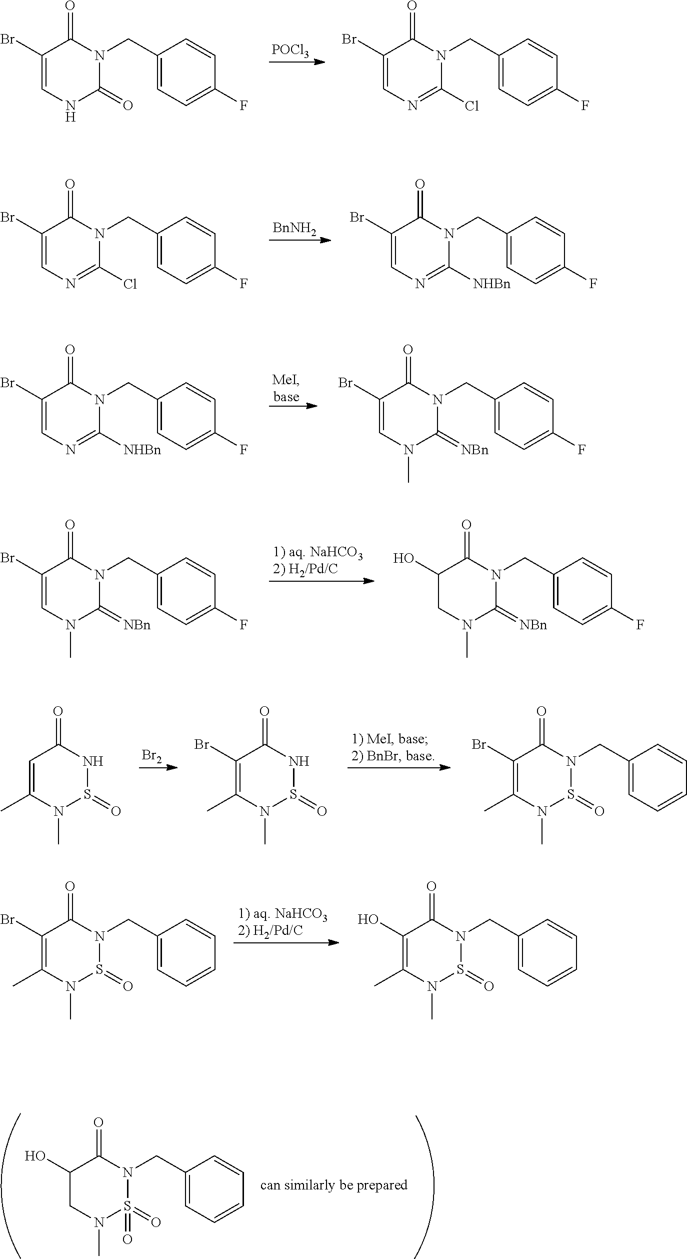

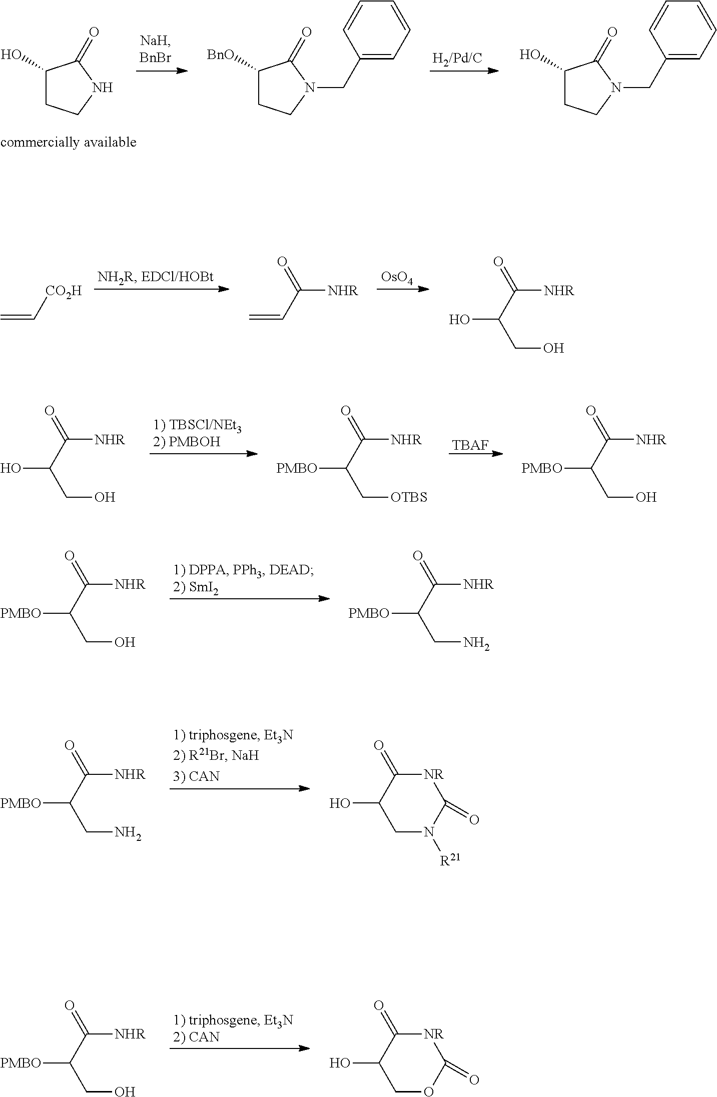

The following compounds can be prepared by the procedures described in the cited references, the disclosures of each reference being incorporated herein by reference thereto:

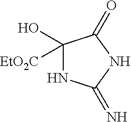

QUIROSA-GUILLOU, C.; RENKO, D. Z.; THAL, C.; Tetrahedron 1992, 48 (31), 6385-6392.

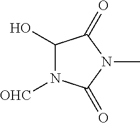

Matsui, M.; Kamiya, K.; Shibata, K.; Muramatsu, H.; J Org Chem 1990, 55, 1396.

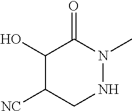

AMRI, H.; EL GAIED, M. M.; AYED, T. B.; VILLIERAS, J.; Tetrahedron Lett 1992, 33 (48), 7345-7346.

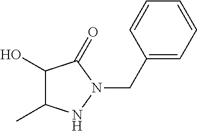

WOYDOWSKI, K.; LIEBSCHER, J.; J Prakt Chem/Chem-Ztg 1998, 340 (6), 567-571.

The invention disclosed herein is exemplified by the following illustrative schemes and examples which should not be construed to limit the scope of the disclosure. Alternative mechanistic pathways and analogous structures will be apparent to those skilled in the art.

The following methods are prophetic and may be used to prepare the indicated compounds:

### Preparative Example 1

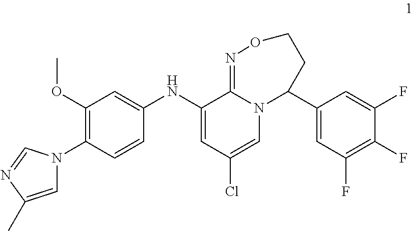

Step A:

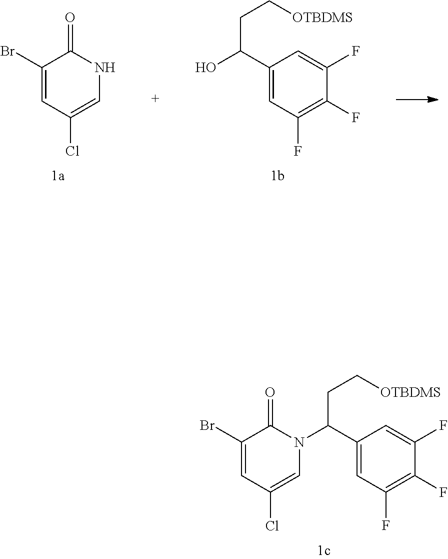

The mixture of 1a (21.8 mmol) and 1b (18.1 mmol) in THF (70 ml) will be stirred at 0° C., PBu₃ (27.2 mmol) will be added dropwise to the mixture, the mixture will be stirred at 0° C. for 0.5 h before the addition of ADDP (6.3 g, 27.2 mmol). The resultant mixture will be kept stirring at 0° C. for 0.5 h, then slowly warm up to 80° C., and keeps stirring at 80° C. for 48 h. The mixture will be cooled to room temperature, the white precipitate will be filtered off, the filtrate will be concentrated and purified via ISCO (EtOAc-Hexane=1:6) to obtain 1c.

Step B:

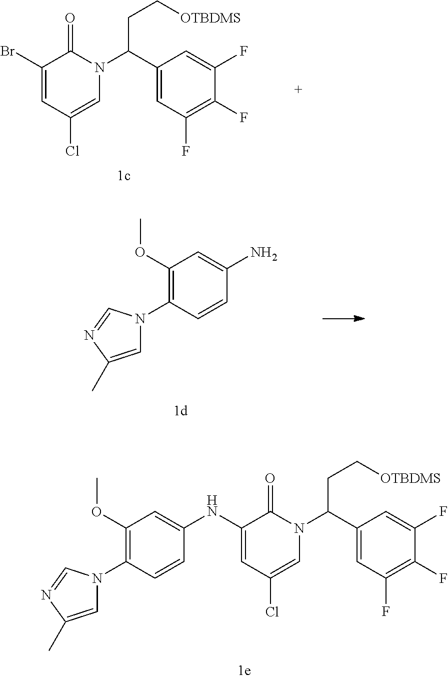

To the mixture of 1c (0.192 mmol) in toluene (10 ml) will be added compound 1d (0.192 mmol) and solid K₂CO₃ (0.960 mmol), followed by the addition of BINAP (4% mol) and Pd(OAc)₂ (4% mol), the resultant mixture will be kept stirring at 1200° C. for 48 h. The mixture will be cooled to room temperature, EtOAc (10 ml) and NH₄Cl (6 ml) will be added, the insoluble material will be filtered off thru Celite. The filtrate will be separated; the aqueous will be extracted once more with EtOAc (6 ml). The combined organic will be dried over anhydrous MgSO₄, and concentrated. The residue will be purified via Gilson to obtain 1e.

Step C:

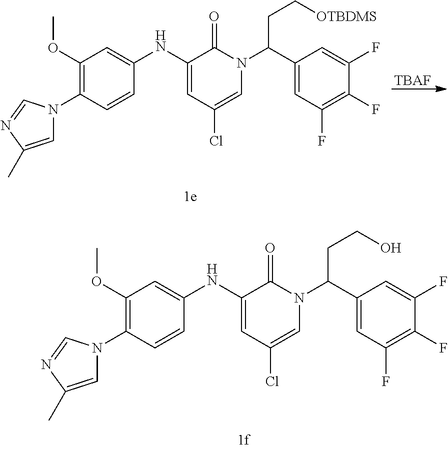

Compound 1e in THF will be treated with TBAF (1.5 eq.) for 2 hour. The mixture will be diluted with EtOAc and NH₄Cl. The aqueous will be extracted once more with EtOAc. The combined organic will be dried over anhydrous MgSO₄, and concentrated. The residue will be purified via Gilson to obtain 1f.

Step D:

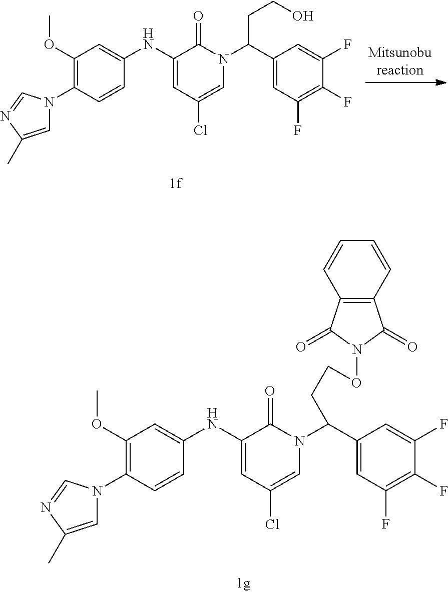

The mixture of 1f (1 mmol) and hydroxyl phthalimide (1.4 mmol) in THF (10 ml) will be stirred at 0° C., PBu₃ (1.5 mmol) will be added dropwise to the mixture, the mixture will be stirred at 0° C. for 0.5 h before the addition of ADDP (1.5 mmol). The resultant mixture will be kept stirring at 0° C. for 0.5 h, then slowly warm up to 80° C., and will be kept stirring at 80° C. for 2 h. The mixture will be cooled to room temperature, the white precipitate will be filtered off, the filtrate will be concentrated and purified via ISCO (EtOAc-Hexane=1:6) to obtain 1g.

Step E:

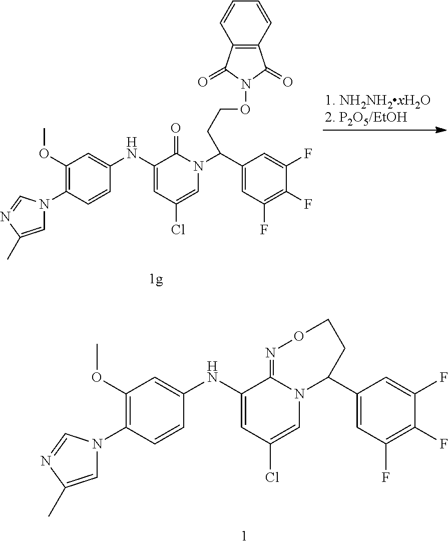

Compound 1g (1.0 mmol) in CH₂Cl₂/MeOH (5 mL/5 mL) will be treated with NH₂NH₂.xH₂O (2.5 mmol) for 1 hour. The mixture will be diluted with EtOAc and 1 N NaOH. The aqueous will be extracted once more with EtOAc. The combined organic will be dried over anhydrous MgSO₄, and concentrated. The residue will be taken up in EtOH and treated with P₂O₅ (10 eq.). The mixture will be heated at reflux overnight before it will be cooled and diluted with EtOAc and 1 N NaOH. The basified aqueous phase will be extracted once more with EtOAc. The combined organic will be dried over anhydrous MgSO₄, and concentrated. The residue will be purified via Gilson to obtain 1.

### Preparative Example 2

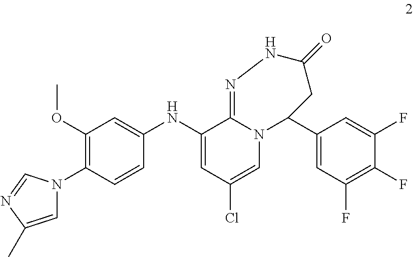

Step A:

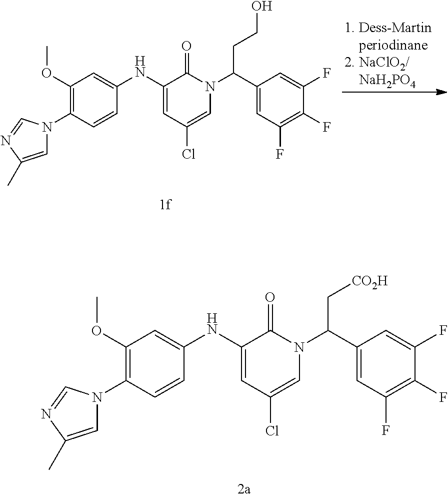

Compound 1f in CH₂Cl₂ will be treated with Dess-Martin Periodinane (1.5 eq) and the mixture will be stirred for 2 hours. The mixture will be diluted with EtOAc and NaHCO₃ solution. The aqueous will be extracted once more with EtOAc. The combined organic will be dried over anhydrous MgSO₄, and concentrated. The residue aldehyde will be taken up in t-BuOH and treated with NaClO₂ (2 eq), NaH₂PO₄ (3.0 eq.). The mixture will be stirred for 2 hours. The mixture will be diluted with EtOAc and NH4Cl solution. The aqueous will be extracted once more with EtOAc. The combined organic will be dried over anhydrous MgSO₄, and concentrated to give 2b.

Step B:

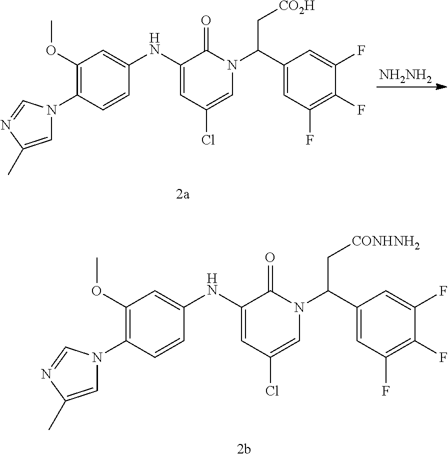

Compound 2a in DMF will be treated with NH₂NH₂ (1.5 eq), EDCl (2.0 eq.), HOBt (2.0 eq.) and iPr₂NEt (3.5 eq) and the mixture will be stirred for 24 hours. The mixture will be diluted with EtOAc and NH4Cl solution. The aqueous will be extracted once more with EtOAc. The combined organic will be dried over anhydrous MgSO₄, and concentrated. The residue will be purified via Gilson to obtain 2b.

Step C:

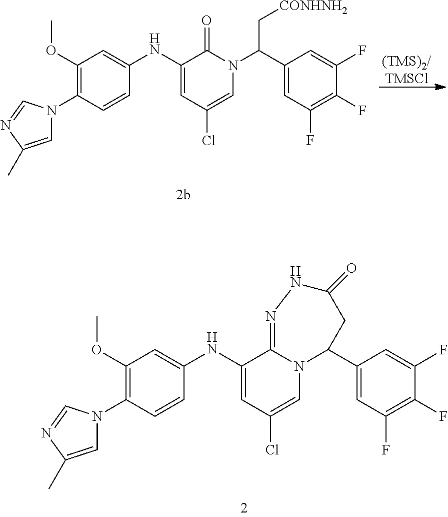

Compound 2b in dioxane will be treated with (TMS)₂/TMSCl (1.5 eq./1.5 eq.) and the mixture will be heated in a sealed tube for 24 hours. The mixture will be diluted with EtOAc and NaHCO₃ solution. The aqueous will be extracted once more with EtOAc. The combined organic will be dried over anhydrous MgSO₄, and concentrated. The residue will be purified via Gilson to obtain 2.

### Preparative Example 3

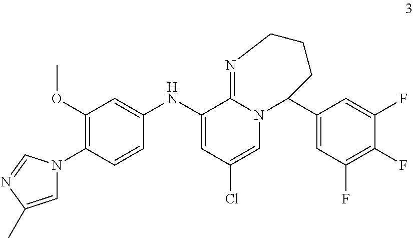

Step A

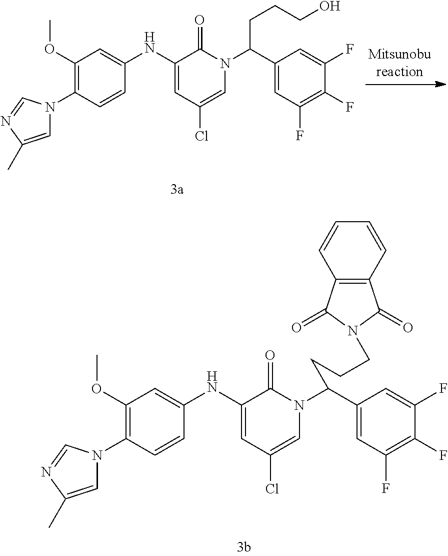

Compound 3a will be prepared in the similar route of compound 1f.

The mixture of 3a (1 mmol) and phthalimide (1.4 mmol) in THF (10 ml) will be stirred at 0° C., PBu₃ (1.5 mmol) will be added dropwise to the mixture, the mixture will be stirred at 0° C. for 0.5 h before the addition of ADDP (1.5 mmol). The resultant mixture will be kept stirring at 0° C. for 0.5 h, then slowly warm up to 80° C., and keeps stirring at 80° C. for 2 h. The mixture will be cooled to room temperature, the white precipitate will be filtered off, the filtrate will be concentrated and purified via ISCO (EtOAc-Hexane=1:6) to obtain 3b.

Step B:

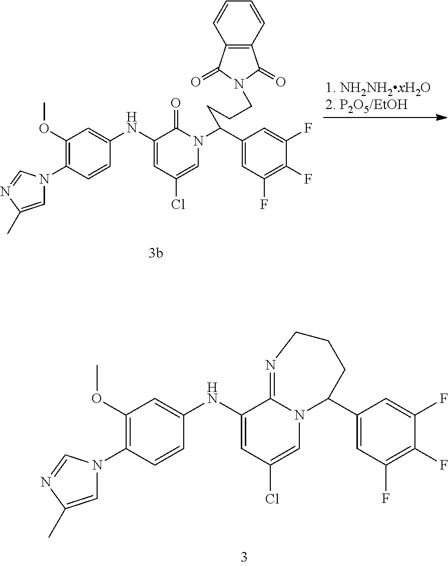

Compound 3b (1.0 mmol) in CH₂Cl₂/MeOH (5 mL/5 mL) will be treated with NH₂NH₂.xH₂O (2.5 mmol) for 1 hour. The mixture will be diluted with EtOAc and 1 N NaOH. The aqueous will be extracted once more with EtOAc. The combined organic will be dried over anhydrous MgSO₄, and concentrated. The residue will be taken up in EtOH and treated with P₂O₅ (10 eq.). The mixture will be heated at reflux overnight before it will be cooled and diluted with EtOAc and 1 N NaOH. The basified aqueous phase will be extracted once more with EtOAc. The combined organic will be dried over anhydrous MgSO₄, and concentrated. The residue will be purified via Gilson to obtain 3.

### Preparative Example 4

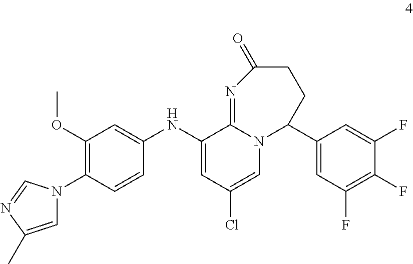

Step A:

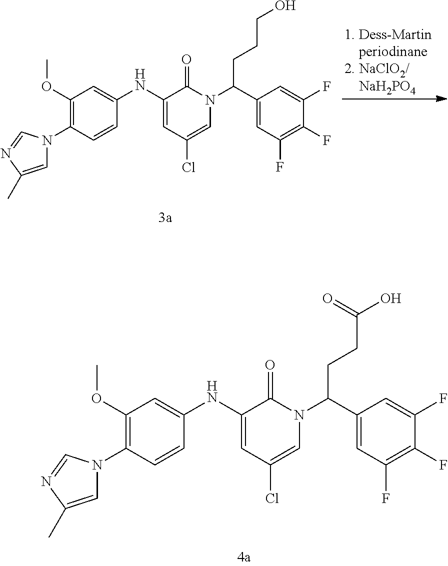

Compound 3a in CH₂Cl₂ will be treated with Dess-Martin Periodinane (1.5 eq) and the mixture will be stirred for 2 hours. The mixture will be diluted with EtOAc and NaHCO₃ solution. The aqueous will be extracted once more with EtOAc. The combined organic will be dried over anhydrous MgSO₄, and concentrated. The residue aldehyde will be taken up in t-BuOH and treated with NaClO₂ (2 eq), NaH₂PO₄ (3.0 eq.). The mixture will be stirred for 2 hours. The mixture will be diluted with EtOAc and NH4Cl solution. The aqueous will be extracted once more with EtOAc. The combined organic will be dried over anhydrous MgSO₄, and concentrated to give 4a.

Step B:

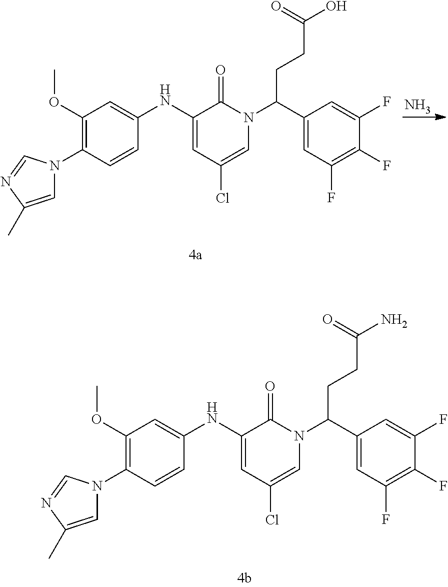

Compound 4a in DMF will be treated with NH₃ (1.5 eq), EDCl (2.0 eq.), HOBt (2.0 eq.) and iPr₂NEt (3.5 eq) and the mixture will be stirred for 24 hours. The mixture will be diluted with EtOAc and NH4Cl solution. The aqueous will be extracted once more with EtOAc. The combined organic will be dried over anhydrous MgSO₄, and concentrated. The residue will be purified via Gilson to obtain 4b.

Step C:

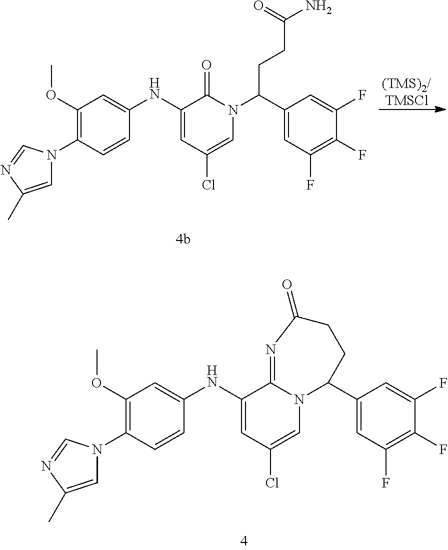

Compound 4b in dioxane will be treated with (TMS)₂/TMSCl (1.5 eq./1.5 eq.) and the mixture will be heated in a sealed tube for 24 hours. The mixture will be diluted with EtOAc and NaHCO₃ solution. The aqueous will be extracted once more with EtOAc. The combined organic will be dried over anhydrous MgSO₄, and concentrated. The residue will be purified via Gilson to obtain 4.

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-WO2 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody WO2 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1-42. (canceled)

43. A compound of the Formula I: 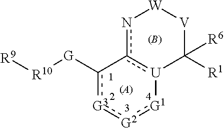
or a pharmaceutically acceptable salt or solvate thereof, wherein:
G, G¹, G², G³, R¹, R⁹, R¹⁰, R²¹, U, V and W are each independently selected;
(A) and (B) in the rings in Formula 1 are reference letters to identify the ring;
either ring (A) is a 5 to 8-membered aryl-, heteroaryl-, cycloalkyl-, cycloalkenyl-, heterocyclyl- or heterocyclenyl-ring, and ring (B) is a 7 or 8-membered heterocyclyl- or heterocyclenyl-ring which contains at least one N atom, or ring (A) is a 7 or 8-membered aryl-, heteroaryl-, cycloalkyl-, cycloalkenyl-, heterocyclyl-, heterocyclenyl-, heterocyclylalkyl- or heterocyclyalkenyl-ring, and ring (B) is a 5 to 8-membered heterocyclyl- or heterocyclenyl-ring which contains at least one N atom;
each dotted line (----) represents an optional bond;
U is selected from the group consisting of: N, C (if the optional double bond attached to U is present), —CH— or —CR²¹—;
G¹ is selected from the group consisting of: a direct bond, —O—, —C(R²¹)₂—,
—CHR²¹—, —CH₂—, —CR²¹— (if the optional double bond to G¹ is present), CH (if the optional double bond to G¹ is present), —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—,
—CH₂—CH₂—, —NR²—, —N═ (if the optional double bond to G¹ is present), —C(O)—,
—C(═NR²)—, —S(O)₂— and —S(O)—, with the proviso that when the optional double bond between (3) and (4) is present then G¹ is not —O—, —C(R²¹)₂—, —C(R²¹)₂—C(R²¹)₂—, —CHR²¹—C(R²¹)₂—, —C(R²¹)₂—CHR²¹—, —CHR²¹—CHR²¹—, —C(R²¹)₂—CH₂—, —CH₂—C(R²¹)₂—, —CH₂—CH₂—, —NR²—, —C(O)—, —C(═NR²)—, —S(O)₂—, or —S(O)—;
G² is selected from the group consisting of: —O—, —C(R²¹)₂—, —CHR²¹—, —CH₂—, —CR²¹— (if the optional double bond to G² is present), CH (if the optional double bond to G² is present), —C(R²¹)₂—C(R²¹)₂—, —CHR²¹—C(R²¹)₂—, —C(R²¹)₂—CHR²¹—,
—CHR²¹—CHR²¹—, —C(R²¹)₂—CH₂—, —CH₂—C(R²¹)₂—, —CH₂—CH₂—, —NR²—, —N═ (if the optional double bond to G² is present), —C(O)—, —C(═NR²)—, —S(O)₂— and —S(O)—, with the proviso that when the optional double bond between (3) and (4) is present then G² is not —O—, —C(R²¹)₂—, —CHR²¹—, —CH₂—, —C(R²¹)₂—C(R²¹)₂—, —CHR²¹—C(R²¹)₂—, —C(R²¹)₂—CHR²¹—,
—CHR²¹—CHR²¹—, —C(R²¹)₂—CH₂—, —CH₂—C(R²¹)₂—, —CH₂—CH₂—, —NR²—, —C(O)—, —C(═NR²)—, —S(O)₂—, or —S(O)—;
G³ is selected from the group consisting of: —C(R²¹)₂—, —CHR²¹—,
—CH₂—, —CR²¹— (if the optional double bond to G³ is present), CH (if the optional double bond to G³ is present), —NR²—, and —N═ (if the optional double bond to G³ is present), with the proviso that when the optional double bond between (1) and (2) is present then G³ is not —C(R²¹)₂—, —CHR²¹—, —CH₂—, or —NR²—;
W is selected from the group consisting of: —O—, —S—, —C(R²¹)₂—, —CHR²¹—,
—CH₂—, —C(R²¹)₂—C(R²¹)₂—, —CHR²¹—C(R²¹)₂—, —C(R²¹)₂—CHR²¹—, —CHR²¹—CHR²¹—,
—C(R²¹)₂—CH₂—, —CH₂—C(R²¹)₂—, —CH₂—CH₂—, —NR²—, —C(O)—, —C(═NR²)—, —S(O)₂— and
—S(O)—;
V is selected from the group consisting of: a direct bond, —O—, —C(R²¹)₂—,
—CHR²¹—, —CH₂—, —C(R²¹)₂—C(R²¹)₂—, —CHR²¹—C(R²¹)₂—, —C(R²¹)₂—CHR²¹—,
—CHR²¹—CHR²¹—, —C(R²¹)₂—CH₂—, —CH₂—C(R²¹)₂—, —CH₂—CH₂—, —NR²—, —C(O)—,
—C(═NR²)—, —S(O)₂— and —S(O)—;
G is selected from the group consisting of: —C(O)—, —(C═NR²)—, —(C═C(R⁶)₂)—, —C(R⁴)₂—, —CF₂—, —N(R²)— (and in one example, —NH—), —O—, —S—, —S(O)ₜ, —CR⁴(OH)—, —CR⁴(OR⁴)—, —C═C—, alkynyl, —C(O)NR⁵—, —O—C(O)—NR⁵—, —NR⁵C(O)—, —NR⁵S(O)ₜ—, —S(O)ₜNR⁵—, —NR⁵—C(O)—O—, —NR⁵—C(O)—NR⁵—, —NR⁵—S(O)ₜ—NR⁵—, —NR⁵—C(═NR²)—NR⁵—, —NR⁵—C(═NR²)—O—, —O—C(═NR²)—NR⁵—,
—C(R⁴)═N—O—, —O—N═C(R⁴)—, —O—C(R⁴)═N—, —N═C(R⁴)—O—, —(CH₂)₂₋₃— (i.e., 2 to 3
—CH₂— groups), cycloalkyl, and heterocycloalkyl (comprising 1 to 4 heteroatoms independently selected from the group consisting of: —O—, —NR²—, —S—, —S(O)—, and
—S(O)₂);
R¹ is selected from the group consisting of: alkyl, alkenyl, alkynyl, cycloalkyl, cycloalkylalkyl-, heterocyclyl (e.g., heterocycloalkyl), cycloalkenyl, arylalkyl-,
alkylaryl-, aryl (e.g., phenyl), heteroaryl (e.g., pyridyl), heterocyclenyl (i.e., heterocycloalkenyl), fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), and fused heterocycloalkylheteroarylalkyl- (i.e., heterocycloalkylfusedheteroarylalkyl-), and wherein each of said alkyl, alkenyl, alkynyl, cycloalkyl, heterocyclyl, cycloalkenyl, aryl, heteroaryl, heterocyclenyl, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups;
R⁶ is selected from the group consisting of: H, —C(O)R¹⁵, —C(O)OR¹⁵, —C(O)N(R¹⁵)(R¹⁶), —C(═NOR¹⁵)R¹⁶, —CN, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, benzofusedcycloalkyl (i.e., fused benzocycloalkyl), fused benzoheterocycloalkyl, fused heteroarylcycloalkyl, and fused heteroarylheterocycloalkyl; and wherein each of said R⁶ alkyl-, alkenyl-, alkynyl-,
aryl-, arylalkyl-, alkylaryl-, cycloalkyl-, cycloalkylalkyl-, heteroaryl-,
heteroarylalkyl-, heterocyclyl-, heterocyclyalkyl-, benzofusedcycloalkyl, fused benzoheterocycloalkyl, fused heteroarylcycloalkyl, and fused heteroarylheterocycloalkyl group is optionally substituted with 1-5 independently selected R²¹ substituents; or
R¹ and R⁶, taken together with the carbon atom to which they are bound, form a spirocyclic carbocyclic moiety or a spirocyclic heterocyclic moiety, and:
(a) optionally, said spirocyclic carbocyclic moiety is substituted with 1-4 independently selected R²¹ substituents,
(b) optionally, said spirocyclic heterocyclic moiety is substituted with 1-4 independently selected R²¹ substituents,
(c) optionally, said spirocyclic carbocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents,
(d) optionally, said spirocyclic heterocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents;
R² is selected from the group consisting of: H, —OH, —O-alkyl (i.e., alkoxy), —O-(halo substituted alky) (such as, for example, —O-fluoroalkyl), —NH(R⁴), —N(R⁴)₂ (wherein each R⁴ is independently selected), —NH₂, —S(R⁴), —S(O)R⁴, —S(O)(OR⁴),
—S(O)₂R⁴, —S(O)₂(OR⁴), —S(O)NHR⁴, —S(O)N(R⁴)₂ (wherein each R⁴ is independently selected), —S(O)NH₂, —S(O)₂NHR⁴, —S(O)₂N(R⁴)₂ (wherein each R⁴ is independently selected), —S(O)₂NH₂, —CN, —C(O)OR⁴, —C(O)NHR⁴, —C(O)N(R⁴)₂ (wherein each R⁴ is independently selected), —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
R³ is selected from the group consisting of: H, —OH, halo, —O-alkyl (i.e., alkoxy), —O-(halo substituted alkyl), —NH(R⁴), —N(R⁴)₂ (wherein each R⁴ is independently selected), —NH₂, —S(R⁴), —S(O)R⁴, —S(O)(OR⁴), —S(O)₂R⁴,
—S(O)₂(OR⁴), —S(O)NHR⁴, —S(O)N(R⁴)₂, —S(O)NH₂, —S(O)₂NHR⁴, —S(O)₂N(R⁴)₂,
—S(O)₂NH₂, —CN, —C(O)OR⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
each R⁴ is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
each R⁵ is independently selected from the group consisting of: H, unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted aryl, substituted aryl, unsubstituted heteroaryl and substituted heteroaryl; wherein said substituted groups are substituted with 1 to 5 substituents independently selected from R²¹;
R⁹ is selected from the group consisting of: aryl, heteroaryl, heterocyclyl, heterocyclenyl, and heterocyclyalkyl-, wherein each of said R⁹ aryl, heteroaryl, heterocyclyl, heterocyclenyl, or heterocyclyalkyl- is optionally substituted with 1 to 5 independently selected R²¹ groups;
R¹⁰ is selected from the group consisting of: aryl-, heteroaryl-, fused cycloalkylaryl (i.e., cycloalkylfusedaryl-), fused heterocycloalkylaryl- (i.e., heterocycloalkylfusedaryl-), fused cycloalkylheteroaryl- (i.e., cycloalkylfusedheteroaryl-), and fused heterocycloalkylheteroaryl- (i.e., heterocycloalkylfusedheteroaryl-), wherein each of said R¹⁰ moieties is optionally substituted with 1 to 5 independently selected R²¹ groups;
optionally, R⁹ and R¹⁰ can be linked together to form a fused tricyclic alkyl ring, a fused tricyclic heteroalkyl ring, a fused tricyclic aryl ring, a fused tricyclic heteroaryl ring, a fused tricyclic alkenyl ring or a fused tricyclic heteroalkenyl ring;
R¹⁵, R¹⁶ and R¹⁷ are the same or different and each is independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl,
(R¹⁸)ₙ-heteroaryl and (R¹⁸)ₙ-heteroarylalkyl;
R^{15A} and R^{16A} are the same or different and each is independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl,
(R¹⁸)ₙ-heteroaryl and (R¹⁸)ₙ-heteroarylalkyl;
each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN,
—C(O)R¹⁹, —C(O)OH, —C(O)OR¹⁹
—C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹ —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂,
—NHR²⁰, —N(alkyl)₂, —N(arylalkyl)₂, —N(arylalkyl)-(heteroarylalkyl), —NHC(O)R²⁰—NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
optionally, two R¹⁸ moieties on adjacent carbons can be linked together to form a 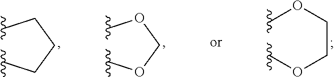
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR⁵,
—C(O)R¹⁵, —C(O)OR¹⁵, —C(O)N(R¹⁵)(R¹⁶), —P(O)(CH₃)₂, —SO(═NR¹⁵)R¹⁶—, —SF₅,
—OSF₅, —Si(R¹⁵)₃ wherein each R¹⁵ is independently selected, —SR¹⁵,
—S(O)N(R¹⁵)(R¹⁶), —CH(R¹⁵)(R¹⁶), —S(O)₂N(R¹⁵)(R¹⁶), —C(═NOR¹⁵)R¹⁶,
—P(O)(OR¹⁵)(OR¹⁶), —N(R¹⁵)(R¹⁶), -alkyl-N(R¹⁵)(R¹⁶), —N(R¹⁵)C(O)R¹⁶—CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}—N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶,
—S(O)R^{15A}, ═NOR¹⁵, —N₃, —NO₂, —S(O)₂R^{15A}, —O—N═C(R⁴)₂ (wherein each R⁴ is independently selected), and —O—N═C(R⁴)₂ (wherein the two R⁴ groups are taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—), wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;
each R²² group is independently selected from the group consisting of alkyl, cycloalkyl, cycloalkenyl, heterocycloalkyl, aryl, heteroaryl, halo, —CF₃, —CN,
—OR¹⁵, —C(O)R¹⁵, —C(O)OR¹⁵, -alkyl-C(O)OR¹⁵, C(O)N(R¹⁵)(R¹⁶), —SF₅, —OSF₅,
—Si(R¹⁵)₃ wherein each R¹⁵ is independently selected, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and
—S(O)₂R^{15A};
optionally, one of (a)-(b) below can represent a spiro ring:
(a) when G¹, G² or G³ represents —C(R²¹)₂—, —C(R²¹)₂—C(R²¹)₂—, —CHR²¹—C(R²¹)₂—, —C(R²¹)₂—CHR²¹—, —C(R²¹)₂—CH₂—, or —CH₂—C(R²¹)₂—, two R²¹ groups on one of the —C(R²¹)₂— groups on one of G¹, G² or G³ can together form a 3 to
8-membered cycloalkyl, cycloalkenyl, heterocycloalkyl, and heterocycloalkenyl spiro ring attached to ring (A), or
(b) when W or V represents —C(R²¹)₂—, —C(R²¹)₂—C(R²¹)₂—, —CHR²¹—C(R²¹)₂—, —C(R²¹)₂—CHR²¹—, —C(R²¹)₂—CH₂—, or —CH₂—C(R²¹)₂—, two R²¹ groups on one of the
—C(R²¹)₂— groups on one of W or V can together form a 3 to 8-membered cycloalkyl, cycloalkenyl, heterocycloalkyl, and heterocycloalkenyl spiro ring attached to ring (B);
d is 0 or 1;
m is 0 to 6;
n is 1 to 5;
p is 0 to 5;
r is 1 to 3;
t is 1 or 2; and
provided that:
(a) rings (A) and (B) do not have two adjacent —O— atoms in the ring; and
(b) rings (A) and (B) do not have two adjacent sulfur groups in the ring (i.e., when there is a —S—, —S(O)— or —S(O)₂ group at one position, then the adjacent positions are not —S—, —S(O)— or —S(O)₂); and
(c) rings (A) and (B) do not have an —O— atom adjacent to a sulfur group (i.e., rings (A) and (B) do not have an —O— atom adjacent to a —S—,
—S(O)— or —S(O)₂); and
(d) when G¹ is N, then G² is not —O—; and
(e) when G¹ is —O—, then G² is not N; and
(f) when G¹ is a direct bond, and G² is —O—, then G³ is not N; and
(g) when G¹ is N, and G³ is N, then G² is not N; and
(h) when G² is N, and G³ is N, then G¹ is not N; and
(i) when G¹ is N, and G² is N, then G³ is not N; and
(j) when the double bond between positions 1 and 2 is not present, then G is not —C(O)—, —(C═NR)— or —(C═C(R⁴)₂)—.

44. The compound of claim 43 having the formula Ia: 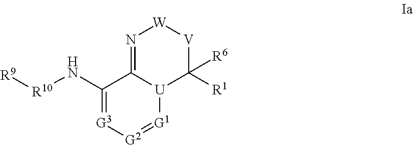
wherein R⁹, R¹⁰, G¹, G², G³, W, V, R⁶ and R¹ are as defined in claim 1.

45. A compound of claim 43 having the formula Ib: 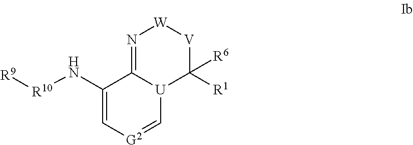
wherein R⁹, R¹⁰, G², W, V, R⁶ and R¹ are as defined above for Formula 1.

46. The compound of claim 43 wherein the R⁹-R¹⁰-moiety is selected from the group consisting of:

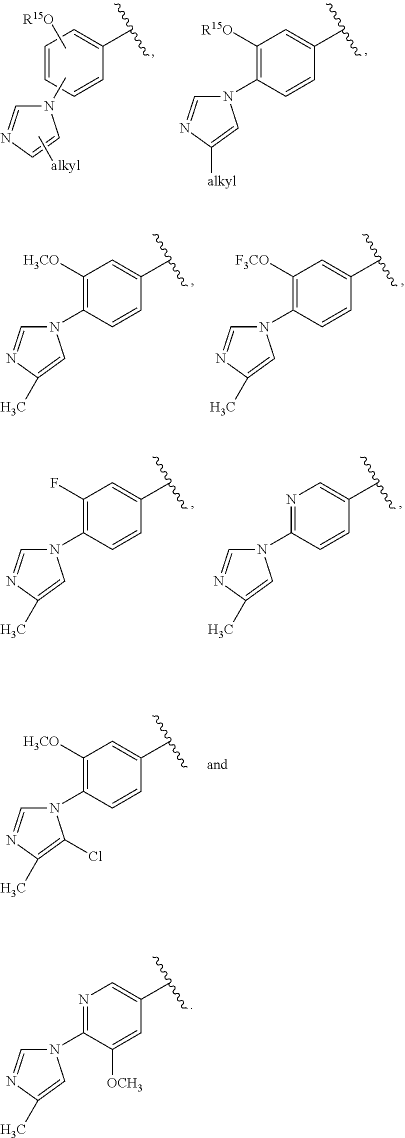

47. The compound of claim 46 wherein G is selected from the group consisting of: —C(O)—, —CH₂—, CF₂, —CH═CH—, —C≡C—, —C(O)NR⁵—, —NR⁵C(O)—,
—(C═NR²)—, —S(O)ₜNR⁵—, —NR⁵S(O)ₜ—, —NH—, —O—, —S—, —S(O)—, —S(O)₂— and a direct bond.

48. The compound of claim 43 wherein R⁶ is H or alkyl.

49. The compound of claim 43 wherein R¹ is selected from the group consisting of:

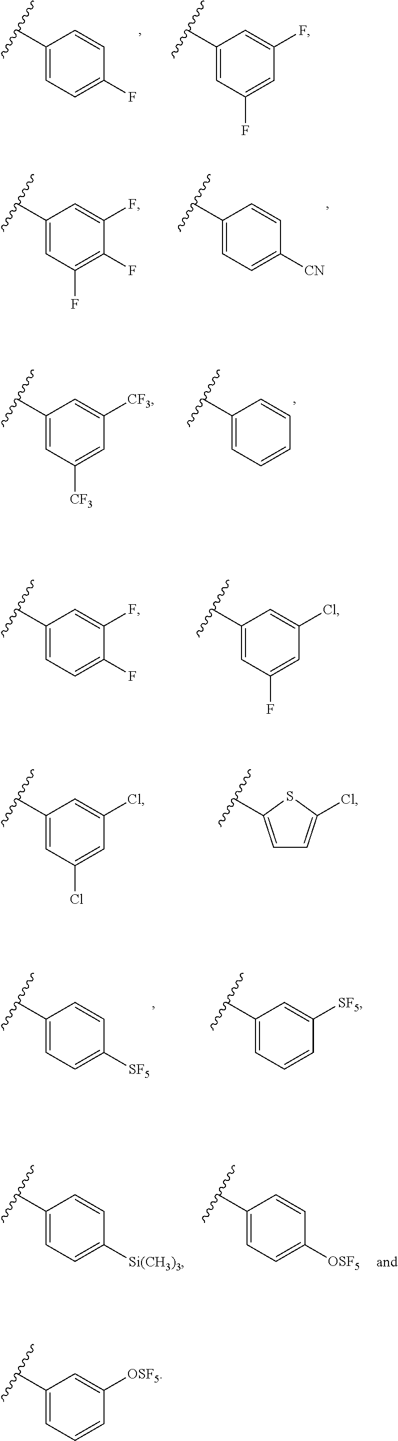

50. The compound of claim 43 selected from the group consisting of 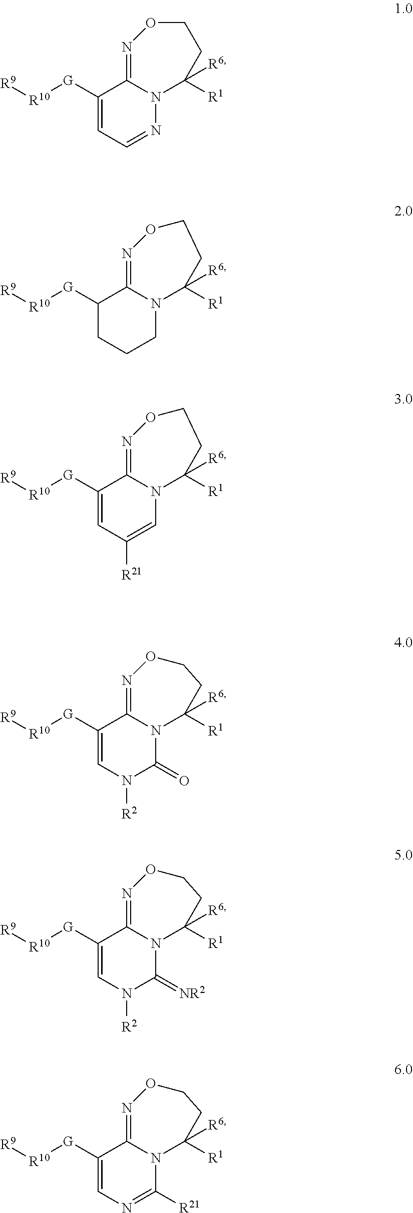 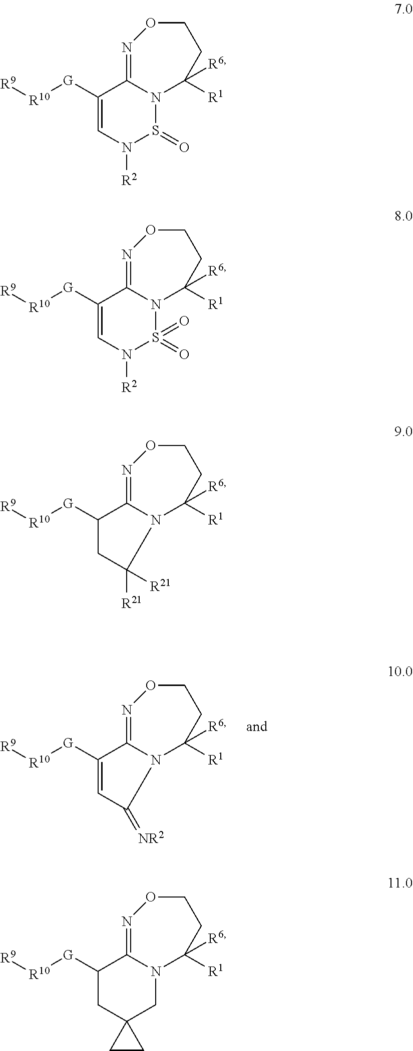
wherein R¹, R², R⁶, R⁹, R¹⁰, R²¹ and G are as defined in claim 43.

51. The compound of claim 50 wherein R¹ is selected from the group consisting of: 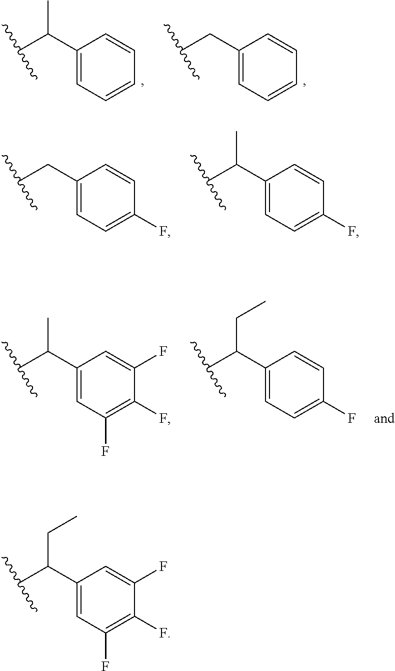 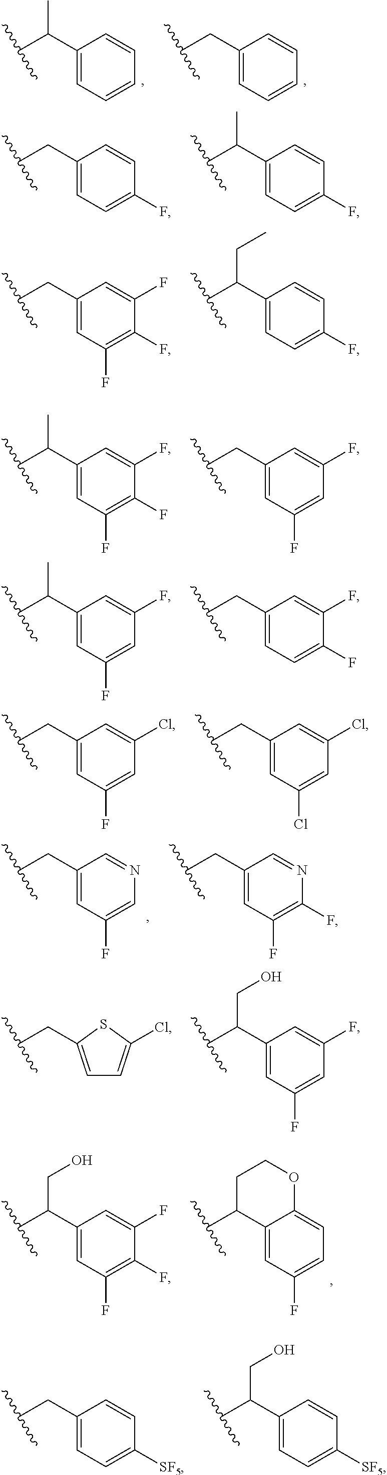 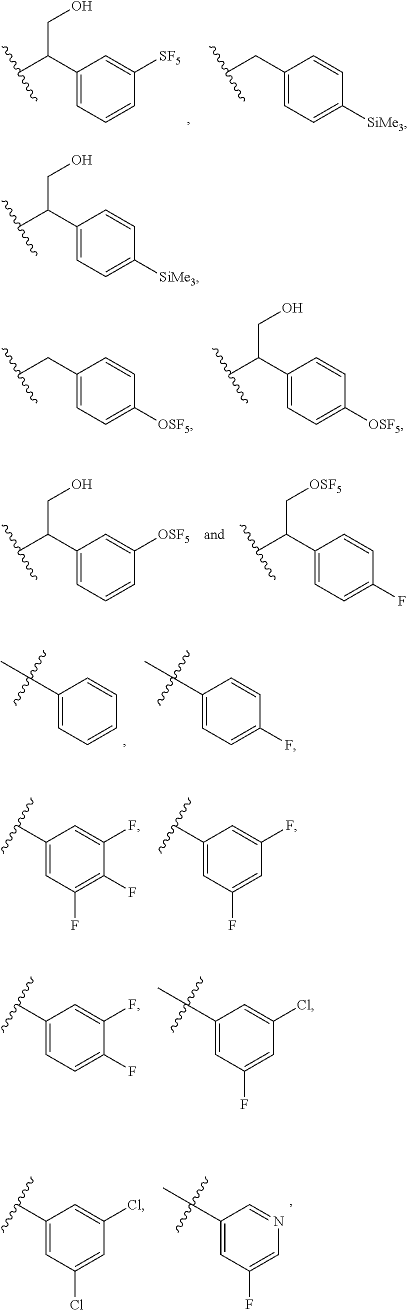 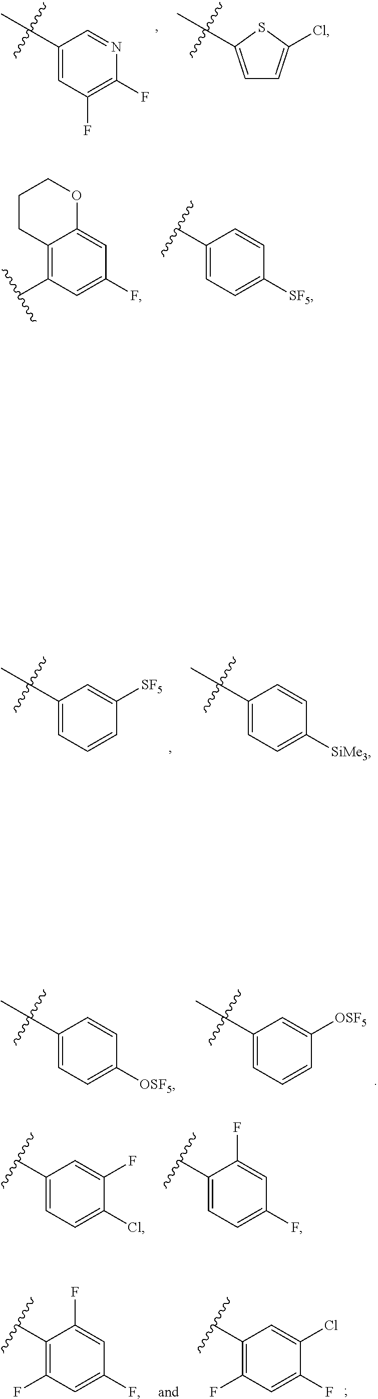
and R⁶ is H or alkyl.

52. The compound of claim 51 wherein G is selected from the group consisting of: —C(O)—, —CH₂—, CF₂, —CH═CH—, —C≡C—, —C(O)NR⁵—, —NR⁵C(O)—,
—(C═NR²)—, —S(O)ₜNR⁵—, —NR⁵S(O)ₜ—, —NH—, —O—, —S—, —S(O)—, —S(O)₂— and a direct bond.

53. The compound of claim 51 wherein: (a) the R⁹-R¹⁰-G moiety is selected from the group consisting of:

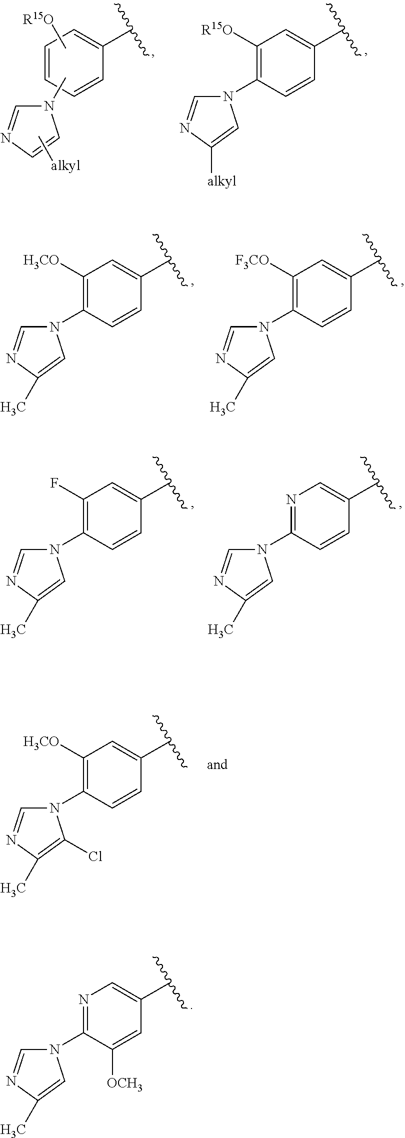

54. The compound of claim 43 selected from the group consisting of 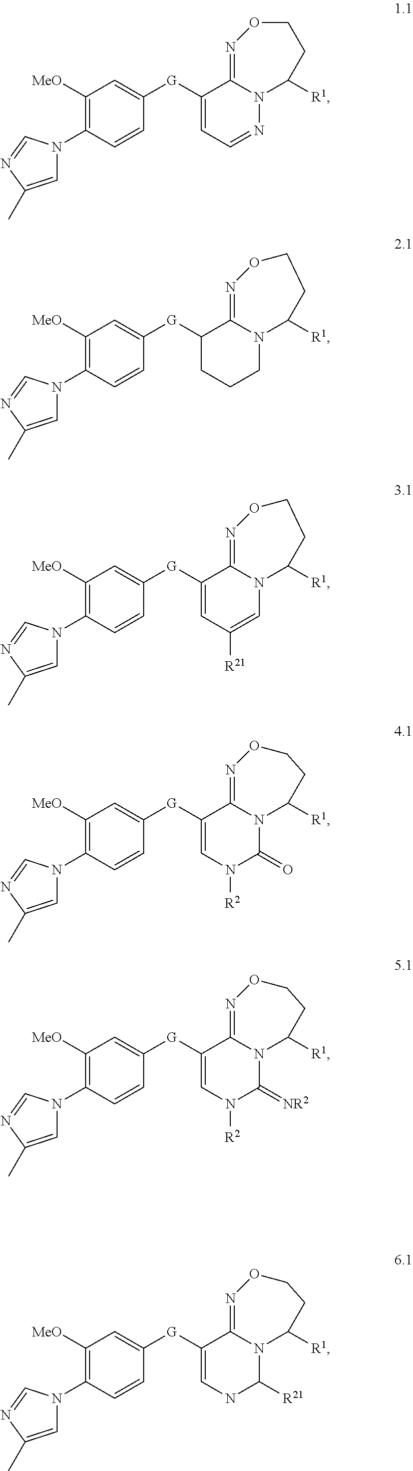 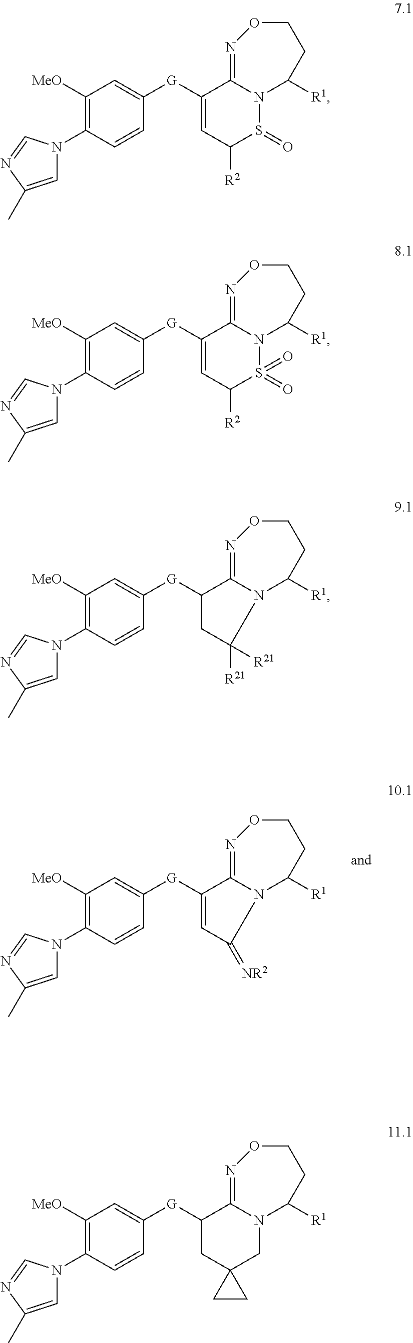
wherein R¹, R², R²¹ and G are as defined in claim 43.

55. The compound of claim 54 wherein R¹ is selected from the group consisting of:

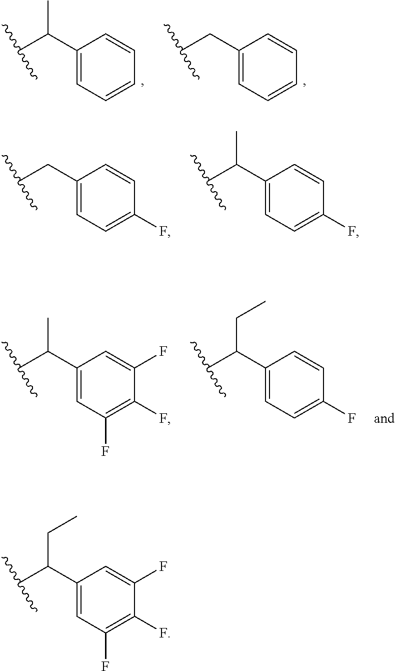

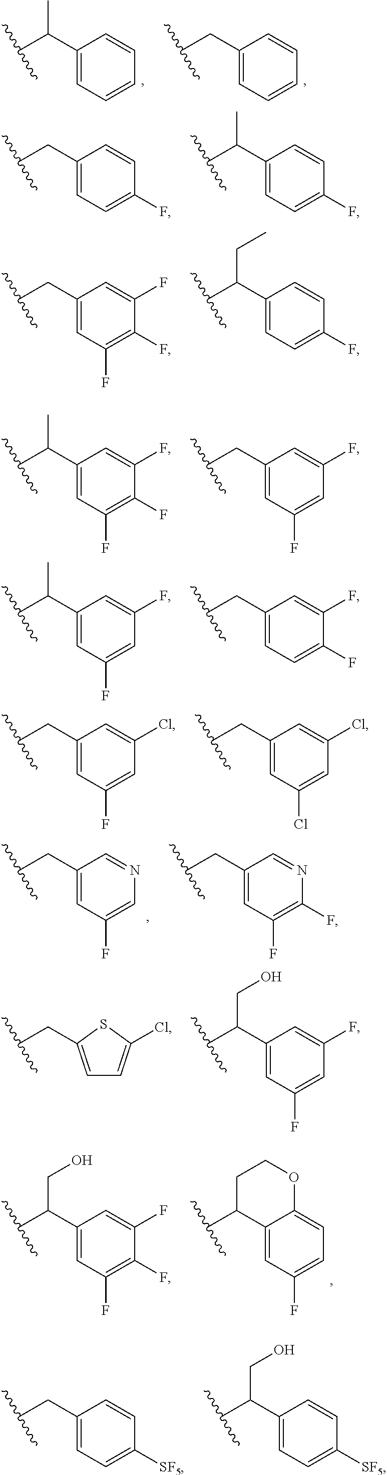

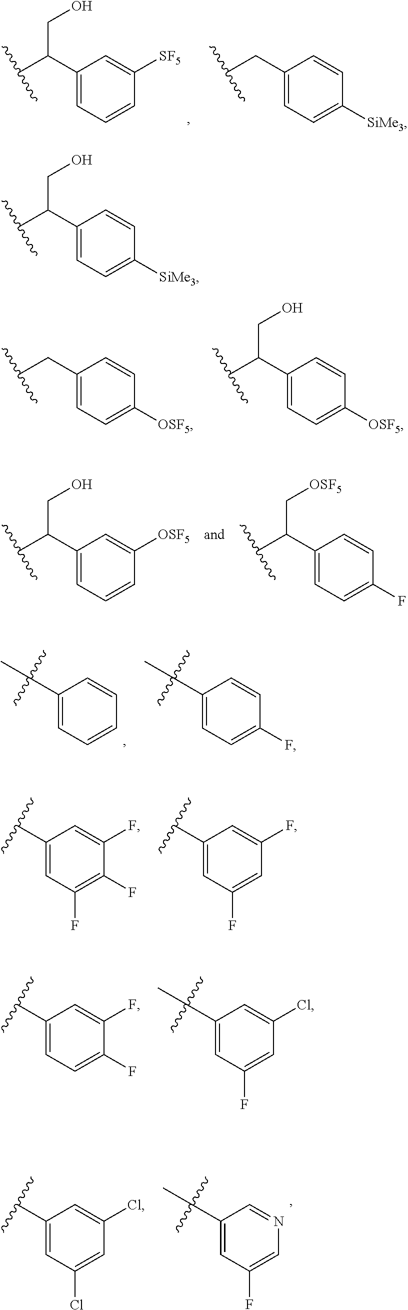

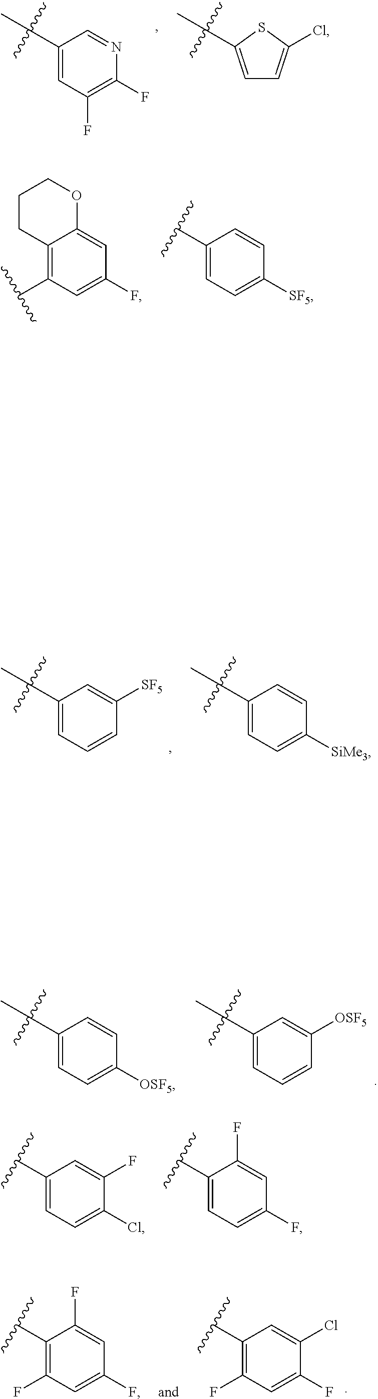

56. The compound of claim 55 wherein G is selected from the group consisting of: —C(O)—, —CH₂—, CF₂, —CH═CH—, —C≡C—, —C(O)NR⁵—, —NR⁵C(O)—,
—(C═NR²)—, —S(O)ₜNR⁵—, —NR SS(O)ₜ—, —NH—, —O—, —S—, —S(O)—, —S(O)₂— and a direct bond.

57. A pharmaceutical composition comprising: a therapeutically effective amount of at least one compound of claim 43, or a pharmaceutically acceptable salt thereof, and at least one pharmaceutically acceptable carrier.

58. A method of treating Alzheimer's disease comprising: administering a therapeutically effective amount of at least one compound of claim 43 to a patient in need of such treatment.
